SEQUENCE LISTING (1 sequence)

SEQ ID NO: 1 - protein, 30 aa, Artificial Sequence
  Synthetic Peptide Biotin and F containing peptide
  MISC_FEATURE (1)..(1): Biotin
  MOD_RES (6)..(6): Azide Modification
  MOD_RES (30)..(30): Azide Modification
  MISC_FEATURE (30)..(30): Amine
KGSGSKGSGSRFAGVAMPGAEDDVVGSGSK

(19) United States
(12) Patent Application Publication
(10) Pub. No.: US 2021/0254047 A1
(43) Pub. Date: Aug. 19, 2021

(54) PROXIMITY INTERACTION ANALYSIS

(71) Applicant: Encodia, Inc., San Diego, CA (US)

(72) Inventors: Mark S. CHEE, San Diego, CA (US); Kevin L. GUNDERSON, San Diego, CA (US)

(73) Assignee: Encodia, Inc., San Diego, CA (US)

(21) Appl. No.: 17/272,236

(22) PCT Filed: Sep. 4, 2019
(86) PCT No.: PCT/US2019/049404

§ 371 Date: Feb. 26, 2021

Related U.S. Application Data
(60) Provisional application No. 62/812,861, filed on Mar. 1, 2019.
(60) Provisional application No. 62/726,933, filed on Sep. 4, 2018.
(60) Provisional application No. 62/726,959, filed on Sep. 4, 2018.

(51) Int. Cl. C12N 15/10

(52) U.S. Cl. CPC C12N 15/1055; C12N 15/1065

(57) ABSTRACT

The present disclosure relates to methods for assessing identity and spatial relationship between a polypeptide and a moiety in a sample. In some embodiments, both the polypeptide and the moiety are parts of a larger polypeptide, and the present methods can be used assess identity and spatial relationship between the polypeptide and the moiety in the same polypeptide or protein. In other embodiments, the polypeptide and the moiety belong to different molecules, and the present methods can be used assess identity and spatial relationship between the polypeptide and the moiety different molecules, e.g., in a protein-protein complex, a protein-DNA complex or a protein-KNA complex.

### RELATED APPLICATIONS

The present application claims priority to U.S. provisional patent application Nos. 62/726,933, filed on Sep. 4, 2018, 62/726,959, filed on Sep. 4, 2018, and 62/812,861, filed on Mar. 1, 2019, the disclosures and contents of which are incorporated by reference in their entireties for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (File name: 4614-2000940_SeqList_ST25_20190829; date recorded; Aug. 29, 2019; size: 1021 bytes).

### TECHNICAL FIELD

The present disclosure relates to methods for assessing identity and spatial relationship between a polypeptide and a moiety in a sample. In some embodiments, both the polypeptide and the moiety are parts of a larger polypeptide, and the present methods can be used to assess identity and spatial relationship between the polypeptide and the moiety in the same polypeptide or protein. In other embodiments, the polypeptide and the moiety belong to different molecules, and the present methods can be used to assess identity and spatial relationship between the polypeptide and the moiety in different molecules, e.g., in a protein-protein complex, a protein-DNA complex or a protein-RNA complex.

### BACKGROUND

Proteins play key roles in cellular and organismal physiology. Proteomics is the study of proteins at a global level including measuring protein abundance, protein interactions, and protein modifications. These protein measurements elucidate how proteins are used within cells, within tissues, and within an organism. I′vloreover, identification of protein markers within a tissue, or a body fluid such as blood or plasma, can serve as a prognostic or diagnostic assay reflective of a particular disease or disorder state, and provide a means to monitor the progression of disease or disorder. Measurement of proteins within plasma is particularly useful since the blood bathes most tissues in the body, picking up potential protein biomarkers from cells and tissues throughout the body. A major challenge in proteomics is that global analysis of proteins is difficult and current tools are largely inadequate. Moreover, the most prevalent method of proteomics analysis, bottom-up peptide sequencing with mass spectrometry, first dicests intact polypeptides into peptides, which are subsequently analyzed in LC-MS/MS. The digestion of polypeptides into peptides disrupts protein-protein interactions, and destroys single molecule information about the precise combinatorial identity of post translational modification (PTM) on a given molecule, i.e., proteoform information is destroyed. Top down mass spectrometry has been utilized to resolve proteoforms, but still has a number of limitations (Kilpatrick and Kilpatrick 2017). As such, there is need for a robust technology to preserve both information on protein-protein interactions, and information on single molecule proteoforrns (particular combination of PTMs on a given molecule).

Accordingly, there remains a need in the art for improved techniques relating to assessing or analyzing identity and spatial relationship between a polypeptide and a moiety in a sample. The present disclosure fulfills these and other related needs.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, the present disclosure provides a method for assessing identity and spatial relationship between a polypeptide and a moiety in a sample, which method comprises: a) forming a linking structure between a site of a polypeptide in a sample and a site of a moiety in said sample, said linking structure comprising a polypeptide tag associated with said site of said polypeptide and a moiety tag associated with said site of said moiety, wherein said polypeptide tag and said moiety tag are associated; b) transferring information between said associated polypeptide tag and said moiety tag or ligating said associated polypeptide tag and said moiety tag to form a shared unique molecule identifier (UMI) and/or barcode; c) breaking said linking structure via dissociating said polypeptide from said moiety and dissociating said polypeptide tag from said moiety tag, while maintaining association between said polypeptide and said potypeptide tag, and maintaining association between said moiety and said moiety tag; and d) assessing said polypeptide tag and at least a partial sequence of said polypeptide, and assessing said moiety tag and at least a partial identity of said moiety, wherein said assessed portions of said polypeptide tag and said moiety tag comprise said shared unique molecule identifier (UMI) and/or barcode indicates that said site of said polypeptide and said site of said moiety in said sample are in spatial proximity.

In one aspect, the present disclosure provides a method for assessing identity and spatial relationship between a polypeptide and a moiety in a sample, which method comprises: a) providing a pre-assembled structure comprising a shared unique molecule identifier (UMI) and/or barcode in the middle portion flanked by a polypeptide tag on ono side and a moiety tag on the other side; b) forming a linking structure between a site of a polypeptide in a sample and a site of a moiety, in said sample by associating said polypeptide tag of said pre-assembled structure to said site of said polypeptide and associating said moiety tag of said pre-assembled structure to said site of said moiety; c) breaking said linking structure via dissociating said polypeptide from said moiety and dissociating said polypeptide tag from said moiety tag, while maintaining association between said polypeptide and said polypeptide tag, and maintaining association between said moiety and said moiety tag; and d) assessing said polypeptide tag and at least a partial sequence of said polypeptide, and assessing said moiety tag and at least a partial identity of said moiety, wherein said assessed portions of said polypeptide tag and said moiety tag comprise said shared unique molecule identifier (UMI) and/or barcode indicates that said site of said polypeptide and said site of said moiety in said sample are in spatial proximity

Also provided herein is a method for assessing identity and spatial relationship between a polypeptide and a moiety in a sample, which method comprises: a) forming a linking structure between a site of a polypeptide in a sample arid a site of a moiety in said sample, said linking structure comprising a polypeptide tag associated with said site of said polypeptide and a moiety tag associated with said site of said moiety, wherein said polypeptide tag and said moiety tag are associated; b) transfeiring information between said associated polypeptide tag and said moiety tag to form a shared unique molecule identifier (UMI) and/or barcode, wherein the shared UMI and/or barcode is formed as a separate record polynucleotide; c) breaking said linking structure via dissociating said polypeptide from said moiety and dissociating said polypeptide tag from said moiety tag, while maintaining association between said polypeptide and said polypeptide tag, and maintaining association between said moiety and said moiety tag; d) assessing said polypeptide tag and at least a partial sequence of said polypeptide, and assessing said moiety tag and at least a partial identity of said moiety; and e) assessing said separate record polytiucleotide to establish the spatial relationship between the site of the polypeptide and the site of the moiety.

In some embodiments, the principles of the present methods and compositions can be applied, or can be adapted to apply, to the polypeptide analysis assays known in the art or in related applications. For example, the principles of the present methods and compositions can be applied, or can be adapted to apply, to the composition, kits and methods disclosed and/or claimed in U.S. Provisional Patent Application Nos. 62/330,841, 62/339,071, 62/376,886, 62/579,844, 62/582,312, 62/583,448, 62/579,870, 62/579,840, 62/582,916, International Patent Application Publication No, WO 2019/089836, WO 2019/089846, WO 2019/089851, and International Patent Application No. PCT/US2017/030702, published as WO 2017/192633 A1.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1 illustrates an exemplary workflow for association by proximity labeling. Proximity of peptide regions within a polypeptide or between associated proteins can be recorded and after digesting into peptide fragments and ProteoCode sequeneina (See e.g., U.S. Provisional Patent Application Nos; 62/330,841, 62/339,071, 62/376,886, 62/579,844, 62/582,312, 62/583,448, 62/579,870, 62/579,840, and 62/582,916, International Patent Application Publication No. WO 2019/089836, WO 2019/089846, WO 2019/089851, and International Patent Application No, PCT/US2017/030702, published as WO 2017/192633 A1), shared UMIs can be used to map “proximal peptides”. (A). A protein sample comprised of a protein complex with P, polypeptide, and M, moiety (in this case another polypeptide), is labeled with DNA tags. (B). Proximal DNA tags (within a polypeptide and between P and M polypeptide units) are allowed to interact and exchange information. In the example shown, primer extension is used to transfer information between proximal tags or from one tag to another. (C). The protein complex is dissociated, and reactive amino acid residues such as cysteines and lysines are capped. (D). The denatured potypeptides are digested with an endoprotease, such as Trypsin. (E). The resultant peptide fragments are comprised of various types of fragments ineluding peptides labeled with proximity recording tags (rTags) containing shared UMI information, peptides labeled with recording tags (w/o shared UMI information), and unlabeled peptides. (F). The rTag-labeled peptides are immobilized onto the appropriate sequencing substrate for ProtoCode peptide sequencing. (G). ProteoCode peptide sequencing is completed, and proximity associated peptides determined by identifying shared UMI sequences.

FIG. 2 illustrates exemplary formats and design of proximity encoding tags. (A). DNA proximity encoding tags for twosided proximity extension encoding. (B). DNA proximity encoding tags for one-sided proximity extension encoding. (C). DNA proximity encoding tags far proximity ligation encoding. (D). DNA proximity encoding lags for proximity ligation (alternate format with exogenous UMI sequence). (E). A DNA tag comprising a UMI is attached to P (or M). A complementary primer to the 3′ portion of the DNA tag is hybridized to the P-attached DNA tag. The complementtiry tag contdris an optional UMI and a conjugating functional element (in the example shown, BP—benzo phenone). The BP element attaches to the M region, and a subsequent primer extension step transfers the UMI information. A similar sequence of events of hybridization or ligation followed by functional conjugation to M can be used for scenarios 2B-D. (F). Multipoint attaclunent diagram. The DNA tags can be pre-hybridized before conjugation to the P-M complex, or can be conjugated first and then hybridized. Information is transferred from the P tag to the two M-tags by primer extension. Other methods can also be used including ligation, both double and single stranded ligation.

FIG. 3 illustrates exemplary proximity encoding of macromolecule and macromolecule complexes via DNA tagging and proximity extension. (A). DNA tags with embedded bartiodes/UMIs are attached to a polypeptide molecule. Proximity extension between neighboring DNA tags leads to one way or two way information transfer between the tags (depending on tag design). The net result is that proximal DNA-tagged sites share UMI/barcode information. The polypeptide is then cleaved into peptide fragments, many of which are labeled with DNA tag (B)s containing proximal UMI information. (B). Protein complexes can be labeled with UMI/barcode DNA tags that are allowed to exchange information by proximity extension. The dotted lines illustrate the extended DNA tag containing shared UIMI/barcode information. Shared UMI information can then be used to reconstruct the identity of interacting proteins (i.e., A interacting with B).

FIG. 4 illustrates exemplary proximity encoding of macromolecule and macromolecule complexes via DNA crosslinking of UMI/Barcode containing DNA crosslinkers. (A). DNA crosslinker containing a UMI/barcode sequence and benzophenone (BP) far coupling to the polypeptide backbone. BP DNA crosslinker has crosslinked two proximal sites on polypeptide. BP is shown for illustration purposes (Park, Koh et al. 2016), but any chemical conjugation reagent that reacts with the peptide backbone or amino acid side chains can be used (Hermanson 2013). After cleavage into peptides, a subset of peptides is or are labeled with proximity DNA tags sharing UMI information. (B). DNA crosslinker with UMIs are used to label proximal sites in a protein complex. After labeling, proteins in proximity contain DNA tags sharing UMI information.

FIG. 5 illustrates exemplary sequence design of proximity DNA crosslinkers. Box P and box M, illustrating attachment to P polypeptide and M moiety, respectively, are understood to be present throughout this illustration. (A). Design of DNA tags capable of proximity extension and formatted to serve as a “recording tag” for downstream ProteoCode peptide/protein analysis. (B). The tags shown use BP for labeling peptide sites, but any chemically reactive group to the peptide backbone or peptide amino acid residues can be used. The sequence structure of the double stranded DNA crosslinker is shown with different sequence elements useful for conversion to a recording tag. F1—forward primer sequence with built in restriction enzyme (RE) site, Sp1=Spacer 1 for priming, Sp2—Spacer 2 for priming, UMI=unique molecular identifier, apostrophe denotes complement sequence. The double stranded DNA crosslinking tags are constructed by annealing two oligonucleotides, one containing the UMI, and the other capable of priming on the UMI oligo. A primer extension step writes the UMI to the other strand creating a dsDNA crosslinking tag. A restriction enzyme digest can be used to removing regions of the crosslinked tag to prepare it for “recording tag” format. (C). After the peptides with DNA tags are immobilized on the sequencing substrate, the Sp1 and Sp2 sequence van be converted into an Sp sequence (recording tag structure) for use in an NGPS sequencing assay.

FIG. 6. Design of DNA tags for Direct Chemical Immobilization or Hybridization/Ligation immobilization on Sequencing Substrates. The linker between the DNA tag and the peptide can be attached to the 5′ terminus (A) or via an internal linkage to the DNA (B). In the example shown in C-E, and internal linker is used to enable efficient hybridization of the 5′ phosphorylated end of the DNA tag to DNA hairpin capture probes on the sequencing substrate. (C-E). Peptides with attached DNA tags are annealed to sequencing substrates via immobilized DNA capture probes. After annealing, the DNA recording tag is ligated to the surface capture probe.

FIG. 7 illustrates an exemplary workflow for association by proximity labeling. (A). A protein sample comprised of a protein complex with P, polypeptide, and M, moiety (in this case another polypeptide), is labeled with DNA tags. (B). Proximal DNA tags within a polypeptide and between P and M polypeptide units) are allowed to interact. In the example shown, primer extension is used to transfer information between the polypeptide tag and the moiety tag to generate a separate record polynucleotide. (C). The protein complex is dissociated, and optionally reactive amino acid residues such as cysteines and lysines are capped. (D). The denatured polypeptides are digested with an endoprotease. (E). The resultant peptide fragments are comprised of various types of fragments including peptides labeled with proximity recording tags (rTags) containing shared UMI information, peptides labeled with recording tags (w/o shared UMI information), unlabeled peptides, and separate record polynucleotides. (F). Separate =ord polymicleotide,s are collected and analyzed and the rTag-labeled peptides are immobilized onto the appropriate sequencing substrate for ProteoCode peptide sequencing. (G). ProteoCode peptide sequencing is completed, and proximity associated peptides determined by identifying shared UMI sequences.

FIG. 8 depicts ligation based proximity cycling. The polypeptide and moiety are labeled with DNA tags which are used for primer extension to generate double stranded DNA tag products (FIG. 8A-8B). Ligation thermocycling generates records which provide information on the proximity of the polypeptide to the moieties (FIG. 8C-8D).

FIG. 9A-9C depicts the generation of separate record polynucleotides from the polypeptide tag and from one or more moiety tags. In an exemplary embodiment, the polypeptide is in spatial proximity of a first moiety (M1) and a second moiety 2 (M2). Two or more separate record polynucleotides are formed in pairwise linking structures, which indicates that P is in spatial proximity of M1 and M2. In addition, further separate record polynucleotides between M1 and M3 or M2 and M4 are formed, indicating that M1 and M3; M2 and M4, are in spatial proximity. In some embodiments, the polypeptide and one or more moieties in spatial proximity (e.g. P-M1-M3) is indicated by indirect or overlapping information from one or more separate record polynucleotides (FIG. 9C).

FIG. 10A-10B depict an exemplary model system for labeling proximal molecules and protein analysis. FIG. 10A (top left) shows in schematic form three molecules: DNA1, DNA2, and Peptide (K(Biotin)GSGSK(N3)GSGSRFAGVAMPGAEDDVVGSGS-K(N3)-NH2 as set forth in SEQ ID NO: 1). These components are used in Example 7 to construct a model linking structure between a site of a polypeptide and a site of a moiety. The 5′ end of DNA1 consists of a 24 nt sequence designed to hybridize to DNA1′, a complementary capture sequence attached to beads. UMI-1 is a randomized sequence that functions as a unique molecular identifier; sp is a spacer sequence that is used for attachment of a capping sequence and encoding sequence that enables NGS sequencing; “U” indicates an uracil base that can be cleaved to remove the downstream PEG linker-sp′-UMI-1′-OL′ sequence following information transfer from DNA1 to DNA2. This section is used for information transfer from DNA1 to DNA2 and/or forming a linking structure between DNA1 and DNA2. Removal following transfer eliminates the complementarity created between DNA1 and DNA2 as a result of information transfer, allowing the DNA1-moiety and DNA2-peptide complexes to separate under mild conditions following trypsin cleavage. This enables trypsin cleavage, and subsequent hybridization and ligation of the DNA2-peptide complex to a DNA2′ capture sequence to be carried out under mild, homogeneous conditions. The OL′ sequence at the 3′ end of DNA1 is complementary to OL at the 3′ end of DNA2, enabling polymerase to extend DNA2 using DNA1 as the template. Copying is terminated at the PEG linker. The 5′ end of DNA2 consists of a 24 nt sequence designed to hybridize DNA2′, a complementary capture sequence attached to beads. The peptide contains a single phenylalanine (F) immediately downstream of a single trypsin cleavage site. In this way, trypsin treatment can produce two sub-peptides. For didactic purposes, these are referenced in Example 1 as a model peptide that contains F at the amino-terminus, and a model moiety that contains Biotin attached to a lysine (K) at the N-terminus. DNA1 and DNA2 each contain DBCO (not shown in the schematic) to enable attachment to the N3 (azide) moieties in the Peptide by suitable methods such as click chemistry, as illustrated in the upper middle panel. The upper right and lower left panels illustrate beads containing a mixture of capture sequences for DNA1 and DNA2 (not distinguished in the illustration). In the lower left panel, the DNA1-DNA2-peptide complex is shown captured on the bead via DNA1 capture sequence. Capture via DNA1 and not DNA2 is accomplished by temporarily blocking the DNA2′ capture sequence during this capture step. Following capture of the complex, information transfer takes place by intra molecular extension (i.e. within an individual DNA1-DNA2-peptide complex), as illustrated in the lower middle panel. In the bottom right panel, USER cleavage and washing removes from DNA1 the region of complementarity created by intra molecular extension. This enables the peptide-DNA2 fragment to be released under mild conditions following trypsinization.

FIG. 10B top left recapitulates FIG. 10A bottom right for purposes of continuity. FIG. 10B top middle shows moiety-DNA1 and peptide-DNA2 complexes captured via their respective DNA1′ and DNA2′ capture sequences attached to a solid support. The top right panel and lower middle panel illustrate an encoding process to assess the polypeptide sequence and the moiety, where seqA and seqB identify the moiety (Biotin, “B”) and peptide (phenylalanine, “F”) binding agents respectively. The lower right panel shows the capping step that uses the sp sequence to add R1, a cap sequence, to enable subsequent sequence analysis via NGS.

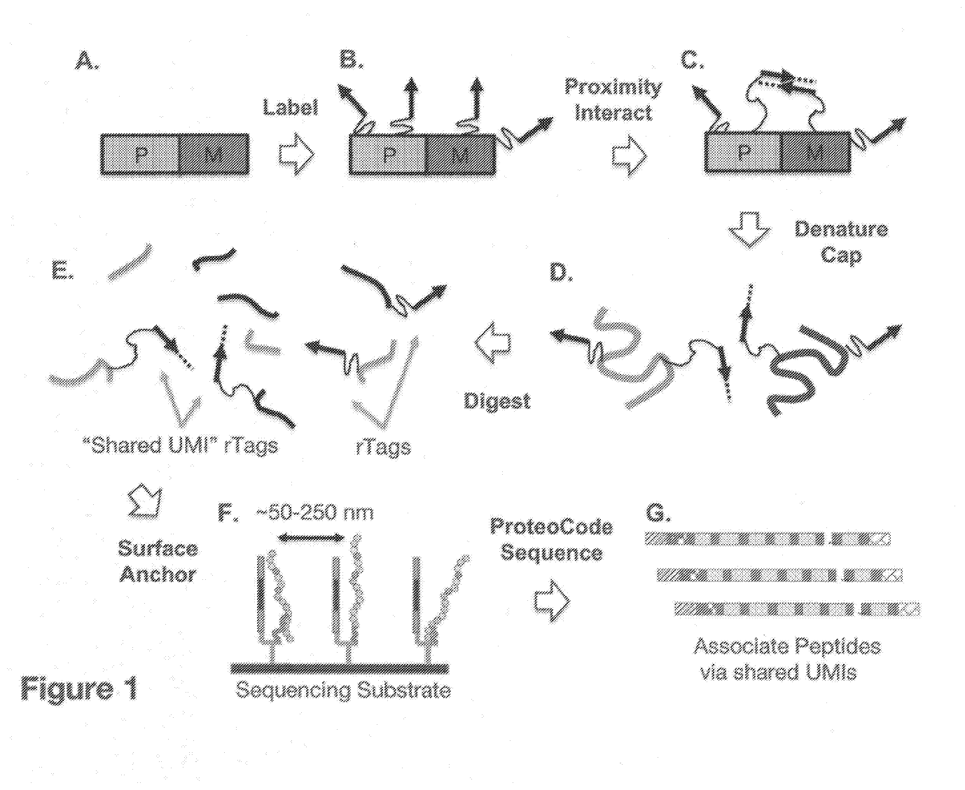

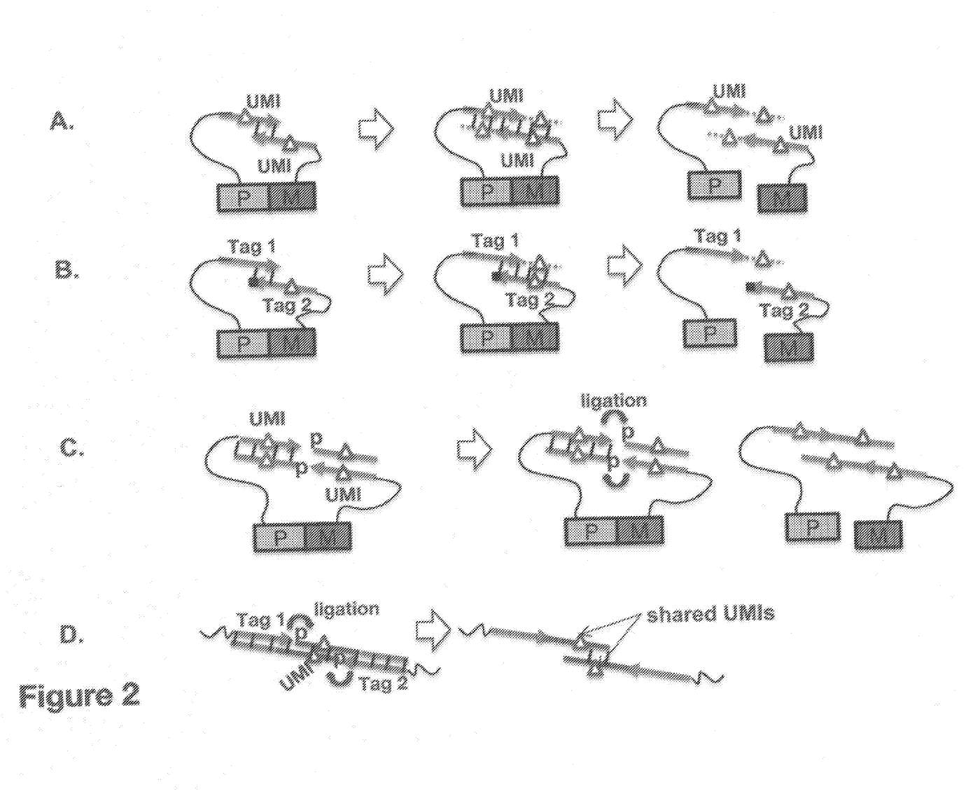

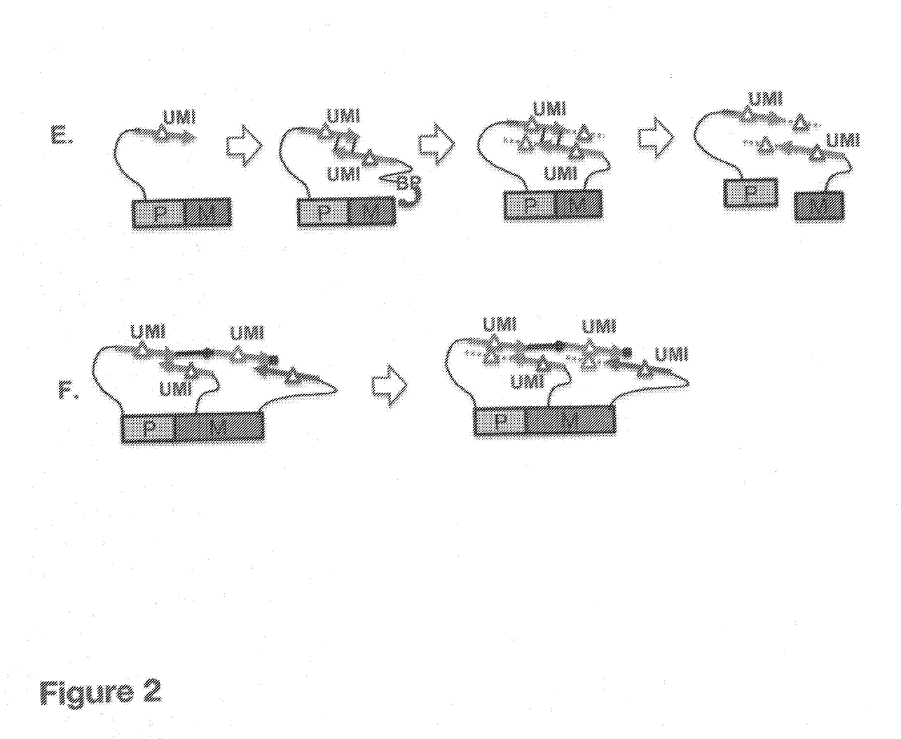

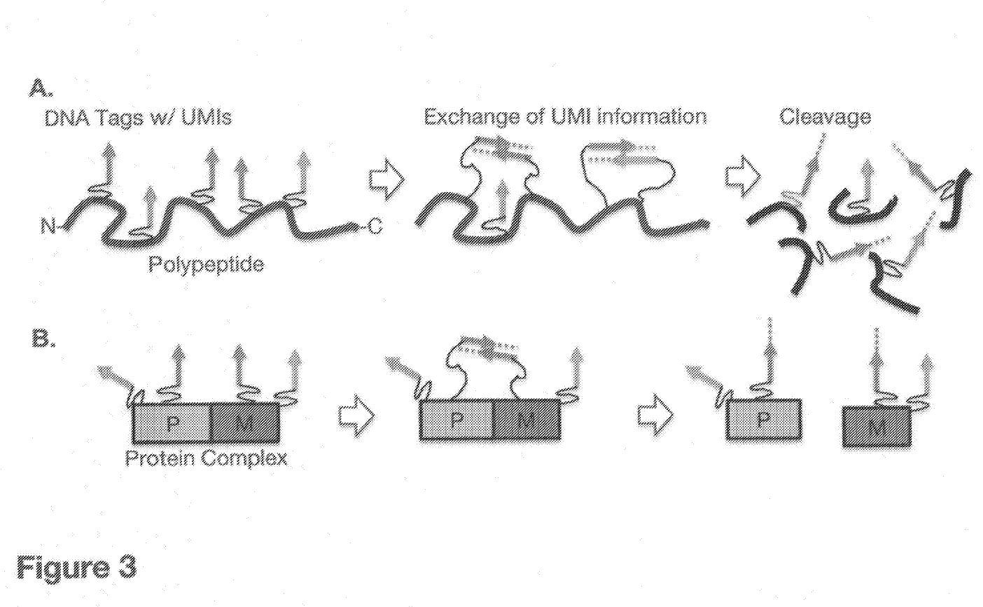

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has i of been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide and/or oligonucleotide synthesis and modification, polymer array synthesis, hybridization and ligation of polynucleotides and/or oligonucleotides, detection of hybridization, and nucleotide sequencing. Specific illustrations of suitable techniques can be had by reference to the examples herein. However, other equivalent conventional procedures can, of course, also be used. Such conventional techniques and descriptions can be found in standard laboratory manuals such as Green, et al., Eds. Genome Analysis: A Laboratrny Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformaties: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al, Biochemistry (2002) 5th Ed., W. H. Freeman Pub., New York. N.Y., all of which are herein incorporated in their entireties by reference for all purposes.

Provided herein are methods and approaches for assessing spatial relationship between a polypeptide and one or more moiety in a sample. In some embodiments, the provided methods further include macromolecule analysis, identification, and/or sequencing. In some embodiments, the spatial relationship between a polypeptide and a moiety is assessed by forming a linking structure between a site of a polypeptide in a sample and a site of a moiety in said sample. In some embodiments, the linking structure comprising a polypeptide tag associated with said site of said polypeptide and a moiety tag associated with said site of said moiety, wherein said polypeptide tag and said moiety tag are associated. In some embodiments, the method also comprises assessing the polypeptide tag and the moiety tag. In some cases, the assessing is for determining the sequence (e.g. partial sequence) of the polypeptide tag and the identity (e.g., partial sequence or identity) of the moiety using a multiplexed macromclecuie binding assay. In some embodiments, the binding assay converts the information from the macromolecule binding assay into a nucleic acid molecule library for readout by next generation sequencing.

Existing methodologies for determireng molecular interactions occurring in biological systems includes imaging and microscopy techniques, for example, Förster or fluorescence resonance energy transfer (FRET) techniques. Other biochemical assays that measure protein interaction include yeast two-hybrid assays, affinity purification assays, mass spectroscopy, and eo-iinmunoprccipitatkm teclmiques. However, there remains a need for improved techniques for assessing spatiai interaction of macromoiecules (e.g., polypeptides or polynucleotides) that are high-throughput, and can detect more than one interaction between various molecules that can also provide the identity/sequence of the molecules to the sample, as well as a need for such products, related methods, and kits for accomplishing the same. In some embodiments, there is a need for technology and methods for assessing identity of molecules and assessing spatial relationships that is accurate, sensitive, and/or high-throughput. In some embodiments, the provided methods allow for assessments, analysis and/or sequencing that overcomes constraints to achieve accurate, sensitive, and/or high-throughput assessment of spatial relationships between molecules and the identity of the molecules (e.g., sequence).

In some cases, the provided, methods allow for identification of the molecules in proximity without the need for specific binding reagents to detect molecular targets for which information regarding the spatial interaction is desired. In some examples, the provided methods for assessing spatial proximity do not require specific target-binding moieties, such as antibodies or binding fragments thereof; to bind to specific molecular targets. In some embodiments, the present disclosure provides, in part, methods for analyzing proximity of molecules (e.g., proteins, polypeptides, moieties), for assessing interactions between molecules, and/or to map interactions between two or more molecules. In some embodiments, the provided methods comprise attachinn of polypeptide tags and moiety tags that are able to bind a variety of polypeptides and moieties. In some embodiments, an exemplary advantage of the provided methods include the ability to assess interactions of numerous molecules (e.g., polypeptides and moieties) in a sample that are in proximity.

In some embodiments, the target polypeptide is a part of a larger polypeptide and the moiety is also part of the same larger polypeptide. In some embodiments, the provided methods are used to analyze a polypeptide and a moiety which are both part of a larger polypeptide and the analysis is useful for applications in sequencing. In some embodiments, the method includes assessing at least a partial sequence of the polypeptide and the moiety. In some cases, the sequence information of the polypeptide and moiety can be used for identifying peptide sequence matches. In some examples, the provided methods allow increased confidence and/or accuracy for sequencing applications, including mapping sequences to polypeptides.

In some embodiments, the provided methods may provide the benefit that shorter and/or less accurate sequences can be used compared to the longer and/or more accurate sequences that may be required using a method for identifying proteins without information of proximal molecules. In some embodiments, the provided methods may be used together with physical partitioning. In some embodiments, the provided methods allow construction of a network using the proximity information such that physical partitioning is not required.

### Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

As used herein, the term “macromolecule” encompasses large molecules composed of smaller subunits. Examples of macromolecules include, but are not limited to peptides, polypeptides, proteins, nucleic acids, carbohydrates, lipids, macrocycles, A macromolecule also includes a chimeric macromolecule composed of a combination of two or more types of macromolecules, covalently linked together (e.g., a peptide linked to a nucleic acid). A macromolecule may also include a “macromolecule assembly”, which is composed of non-covalent complexes of two or more macromolecules. A macromolecule assembly may be composed of the same type of macromolecule (e.g., protein-protein) or of two more different types of macromolecules (e.g., protein-DNA).

As used herein, the term “polypeptide” encompasses peptides and proteins, and refers to a molecule comprising a chain of two or more amino acids joined by peptide bonds. In some embodiments, a polypeptide comprises 2 to 50 amino acids, e.g., having more than 20-30 amino acids. In some embodiments, a peptide does not comprise a secondary, tertiary, or higher structure. In some embodiments, the polypeptide is a protein. In some embodiments, a protein comprises 30 or more amino acids, e.g, having more than 50 amino acids. In some embodiments, in addition to a primary structure, a protein comprises a secondary, tertiary, or higher structure. The amino acids of the polypeptides are most typically 1-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by none amino acids. The term also encompasses an amino acid polymer that has been modified naturally or by intervention; for example, disulfide bond formation, glycosylation, lipidation, acetylation, phosphotylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring amino acids include Alanine (A or Ala), Cysteine (C or Cys), Aspartic Acid (D or Asp), Glutamic Acid (E or Glu), Phenylalanine (F or Phe), Glycine (G o Gly), Histidine (H or His), Isoleucine (I or Ile), Lysine (K or Lys), Leucine (L or Leu), Methionine (M or Met) Asparagine (N of Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). An amino acid may be an L-amino acid or a D-amino acid. Non-standard amino acids may be modified amino acids, amino acid analogs, amino acid mimetics, non-standard proteinogenic amino acids, or non-proteinogertic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids.

As used herein, the term “post-translational modification” refers to modifications that occur on a peptide after its translation by ribosonies is complete. A post-translational modification may be a covalent chemical modification or enzymatic modification. Examples of post-translation modifications include, but are not limited to, acylation, acetylation, alkylation (including methylation), biotinylation, butyrylation, carbamylation, earbonylation, deamidation, deiminiation, diphtbamide formation, disulfide bridge formation, eliminylation, flavin attachment, formylation, gamtna-carboxylation, gititamylation, glycylation glycosylation, glypiation, heme C attachment, hydroxylation, hypusine formation, iodination, isoprenylation, lipidation, malonylation, methylation, myristolylation, oxidation, pahnitoylation, pegylation, phosphopantetheinylation, phosphorylation, prenylation, propionylation, retinylidene Schiff base formation, S-glutathionylation, S-nitrosylation, S-sulfenylation, selenation, succinyiation, sulfinution, uhiquitination, and C-terminal amidation. A post-tarislational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C₁-C₄ alkyl). A post-translational modification also is modifications, such as but not limited to those described above, of amino acids falling between the amino and carboxy termini. The term post-translational modification can also include peptide modifications that include one or more detectable labels.

As used herein, the term “binding agent” refers to a nucleic acid molecule, a peptide, a polypeptide, protein, carbohydrate, or a small molecule that binds to, associates, unites with, recognizes, or combines with a polypeptide or a component or feature of a polypeptide. A binding agent may form a covalent association or non-covalent association with the polypeptide or component or feature of a polypeptide. A binding agent may also be a chimeric binding agent, composed of two or more types of molecules, such as a nucleic acid molecule-peptide chimeric binding agent or a carbohydrate-peptide chimeric binding agent. A binding agent may be a naturally occurring, synthetically produced, or recortibinamly expessed molecule. A binding agent may bind to a single monomer or subunit of a polypeptide (e.g., a single amino acid of a polypeptide) or bind to a plurality of linked subunits of a polypeptide (e.g., a di-peptide tri-peptide, or higher order peptide of a longer peptide, polypeptide, or protein molecule). A binding agent may bind to a linear molecule or a molecule having a three-dimensional structure (also referred to as conformation). For example, an antibody binding agent may bind to linear peptide, polypepetide, or protein, or in to a conformational peptide, polypeptide, protein. A binding agent may bind to an N-terminal peptide, a C-terminal peptide, or an intervening peptide of a peptide, polypeptide, or protein molecule. A binding agent may bind to an N-terminal amino acid, C-terminal amino acid, or an intervening amino acid of a peptide molecule. A binding agent may preferably bind to a chemically modified or labeled amino acid (e.g., an ammo acid that has been functionalized by a reagent comprising a compound of any one of Formula (I)-(VII) as described in International Patent Application No. WO 2019/089846) over a non-modified or unlabeled amino acid. For example, a binding agent may preferably bind to an amino acid that has been fianctionalized with an acetyl moiety, cbz moiety, gtamyl moiety, amino guanidine moiety, dansyl moiety, phenylthiocarbamoyl (PTC) moiety, dinitrophenyl (DN) moiety, sulfonyl nitrophenyl (SNP) moiety, etc., over an amino acid that does not possess said moiety. A binding agent may bind to a post-translational modification of a peptide molecule. A binding agent may exhibit selective binding to a component or feature of a polypeptide (e.g., a binding agent may selectively bind to one of the 20 possible natural amino acid residues and with bind with very low affinity or not at all to the other 19 natural amino acid residues). A binding agent may exhibit less selective binding, where the binding agent is capable of binding a plurality of components or features of a polypeptide (e.g., a binding agent may bind with similar affinity to two or more different amino acid residues). A binding agent comprises a coding tag, which may be joined to the binding agent by a linker.

As used herein, the term “fluorophore” refers to a molecule which absorbs electromagnetic energy at one wavelength and re-emits energy at another wavelength. A fluorophore may be a molecule or part of a molecule including fluorescent dyes and proteins. Additionally, a fluorophore may be chemically, genetically, or otherwise connected or fused to another molecule to produce a molecule that has been “tagged” with the fluorophore.

As used herein, the term “linker” refers to one or more of a nucleotide, a nucleotide analog, an amino acid, a peptide, a polypeptide, or a non-nucleotide chemical moiety that is used to join two molecules. A linker may be used to join a bindina agent with a coding tag, a recording tag with a polypeptide, a polypeptide with a solid support, a recording tag, with a solid support, etc. In certain embodiments, a linker joins two molecules via enzymatic reaction or chemistry reaction (e.g., click chemistry).

The term “ligand” as used herein refers to any molecule or moiety connected to the compounds described herein. “Ligand” may refer to one or more ligands attached to a compound. In some embodiments, the ligand is a pendant group or binding site the site to which the binding agent binds).

As used herein, the term “proteome” can include the entire set of proteins, polypeptides, or peptides (including conjugates or complexes thereof) expressed by a genome, cell, tissue, or organism at a certain time, of any organism. In one aspect, it is the set of expressed proteins in a given type of cell or organism, at a given time, under defined conditions. Proteomics is a the study of the proteome. For example, a “cellular proteome” may include the collection of proteins found in a particular cell type leader a particular set of environmental conditions, such as exposure to hormone stimulation. An organism's complete proteome may include the complete set of proteins from ail of the various cellular proteomes. A proteome may also include the collection of proteins in certain sub-cellular biological systems. For example, all of the proteins in a virus can be called a viral proteome. As used herein, the term “proteome” include subsets of a proteome, including but not limited to a kinome; a secrerome; a receptome (e.g., GPCRome); immunoproteome; a nutriproteome; a proteome subset defined by a post-translational modification (e.g., phosphorylation, ubiquitination. methylation, acetylation, glycosylation, oxidation, lipidation, and/or nitrosylation), such as a phosphoproteome pbosphotyrosine-protecime, tyrosine-kinome, and tyrosine-phosphatome), a glycciprotecime, etc.; a proteome subset associated. with a tissue or organ, a developmental stage, or a physiological Pathological condition; a proteome subset associated a cellular process, such as cell cycle, differentiation (or de-differentiation), cell death, senescence, cell migration, transformation, or metastasis; or any combination thereof. As used herein the term “proteomics” refers to quantitative analysis of the proteonre within cells, tissues, and bodily fluids, and the corresponding spatial distribution o3 the proteome within the cell and within tissues. Additionally, proteomics studies include the dynamic state of the proteome, continually changing in time as a function of biology and defined biological or chemical stimuli.

As used herein, the tem “non-cognate binding agent” refers to a binding adent that is not capable of binding or binds with low affinity to a polypeptide feature, convorient, or subunit being interrogated in a particular binding cycle reaction as compared to a “cognate binding agent”, which binds with high affinity to the corresponding polypeptide feature, component, or subunit. For example, if a tyrosine residue of a peptide molecule is being interrogated in a binding reaction, non-cognate binding agents are those that bind with low affinity or not at all to the tyrosine residue, such that the non-cognate binding agent does not efficiently transfer coding tag information to the recording tag under conditions, that are suitable for transferring coding tag information from cognate binding agents to the recording tag. Alternatively, if a tyrosine residue of a peptide molecule is being interrogated in a binding reaction, non-cognate binding agents are those that bind with low affinity or not at all to the tyrosine residue, such that recording tag information does not efficiently transfer to the coding g under suitable conditions for those embodiments involving extended coding tags rather than extended recording tags.

The terminal amino acid at one end of the peptide chain that has a free amino group is referred to herein as the “N -terminal amino acid” (NTAA). The terminal amino acid at the other end of the chain that has a free carboxyl group is referred to herein as the “C-terminal amino acid” (CTAA). The amino acids making up a peptide may be numbered in order, with the peptide being “n” amino acids in length. As used herein, NTAA is considered the n^{th} amino acid (also referred to herein as the “n NTAA”). Using this nomenclature, the next amino acid is the amino acid, then the n-2 amino acid, and so on dmvn the length of the peptide from the N-terminal end to C-terminal end. In certain embodiments, an NTAA, CTAA, or both may be thnc ona i ed with a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, .18, 19, 20, 21, 22, 23 24, 25, 26, 27, 28, 29 or 30 bases) providing a unique identifier tag or origin information for a polypeptide, a binding agent, a set of binding agents from a binding cycle, a sample polypeptides, a set of samples, polypeptides within a compartment (e.g., droplet, head, or separated location), polypeptides within a set of compartinents, a fraction of polypeptides, a set of polypeptide fractions, a spatial region or set of spatial regions, a library of polypeptides, or a library of binding, agents. A barcode can be art artificial sequence or a naturally occurring sequence. In certain embodiments, each barcode within a population of barcodes is different. In other embodiments, a portion of barcodes in a population of bareodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randonaly generated. In certain embodiments, a population of barcodes are error correcting barcodes. Barcodes can be used to computationally deconvolute the multiplexed sequencing data and identify sequence reads derived from an individual polypeptide, sample, library, etc. A barcode can also be used for deconvolution of a collection of polypeptides that have been distributed into small compartments for enhanced mapping. For example, rather than mapping a peptide back to the proteome, the peptide is mapped back to its originating protein molecule or protein complex.

A “sample barcode”, also referred to as “sample tag” identifies from which sample a polypeptide derives.

A “spatial barcode” identifies which region of a 2-D or 3-D tissue section from which a polypeptide derives. Spatial barcodes may be used for molecular pathology on tissue sections. A spatial barcode allows for multiplex sequencing of a plurality of samples or libraries from tissue section(s).

As used herein, the term “coding tag” refers to a polynucleotide with any suitable length, e.g., a nucleic acid molecule of about 2 bases to about 100 bases, including any integer including 2 and 100 and in between, that comprises identifying information for its associated binding agent. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence, which is optionally flanked by one spacer on one side or flanked by a spacer on each side. A coding tag may also be comprised of an optional UMI and/or an optional binding cycle-specific barcode. A coding tag may be single stranded or double stranded. A double stranded coding tag may comprise blunt ends, overhanging ends, or both. A coding tag may refer to the coding tag that is directly attached to a binding agent, to a complementary sequence hybridized to the coding tag directly attached to a binding agent (e.g., for double stranded coding tags), or to coding tag information present in an extended recording tag. In certain embodiments, a coding tag may farther comprise a binding cycle specific spacer or barcode, a unique molecular identifier, a universal priming site, or any combination thereof.

As used herein, the term “encoder sequence” or “encoder barcode” refers to a nucleic acid molecule of about 2 bases to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) in length that provides identifying information for its associated binding agent. The encoder sequence may uniquely identify its associated binding agent. In certain embodiments, an encoder sequence provides identifying information for its associated binding agent and for the binding cycle in which the binding agent is used. In other embodiments, an encoder sequence is combined with a separate binding cycle-specific barcode within a coding tag. Alternatively, the encoder sequence may identify its associated binding agent as belonging to a member of a set of two or more different binding agents. In some embodiments, this level of identification is sufficient for the purposes of analysis. For example, in some embodiments involving a binding agent that binds to an amino acid, it may be sufficient to know that a peptide comprises one of two possible amino acids at a particular position, rather than definitively identify the amino acid residue at that position. In another example, a common encoder sequence is used for polyclonal antibodies, which comprises a mixture of antibodies that recognize more than one epitope of a protein target, and have varying specificities. In other embodiments, where an encoder sequence identifies a set of possible binding agents, a sequential decoding approach can be used to produce unique identification of each binding agent. This is accomplished by varying encoder sequences for a given binding agent in repeated cycles of binding (see, Gunderson et al., 2004, Genome Res. 14:870-7). The partially identifying coding tag information from each binding cycle, when combined with coding information from other cycles, produces a unique identifier for the binding agent, e.g., the particular combination of coding tags rather than an individual coding tag (or encoder sequence) provides the uniquely identifying information for the binding agent. Preferably, the encoder sequences within a library of binding agents possess the same or a similar number of bases.

As used herein the term “binding cycle specific tag”, “binding cycle specific barcode”, or “binding cycle specific sequence” refers to a unique sequence used to identify a library of binding agents used within a particular binding cycle. A binding cycle specific tag may comprise about 2 bases to about 8 bases (e.g., 2, 3, 4, 5, 6, 7, or 8 bases) in length. A binding cycle specific tag may be incorporated within a binding agent's coding tag as part of a spacer sequence, part of an encoder sequence, part of a UMI, or as a separate component within the coding tag.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag or coding tag. In certain embodiments, a spacer sequence flanks an encoder sequence of a coding tag on one end or both ends. Following binding of a binding agent to a polypeptide, annealing between complementary spacer sequences on their associated coding tag and recording tag, respectively, allows transfer of binding information through a primer extension reaction or ligation to the recording tag, coding tag, or a di-tag construct. Sp′ refers to spacer sequence complementary to Sp. Preferably, spacer sequences within a library of binding agents possess the same number of bases. A common (shared or identical) spacer may be used in a library of binding agents. A spacer sequence may have a “cycle specific” sequence in order to track binding agents used in a particular bnuling cycle. The spacer sequence (Sp) can be constant aeross all binding cycles, be specific for a particular class of polypeptides, or be binding cycle number specific. Polypeptide class-specific spacers permit annealing of a cognate binding agent's coding tag infhmiation present in an extended recording tag from a completed binding/extension cycle to the coding tag of another binding agent recognizing the same class of polypeptides in a subsequent binding cycle via the class-specie spacers. Only the sequential binding of correct cognate pairs results in interacting spacer elements and effective primer extension. A spacer sequence may comprise sufficient number of bases to anneal to a complementary spacer sequence in a recording, tag to initiate a primer extension (also referred to as polymerase extension) reaction, or provide a “splint” for a ligation reaction, or mediate a “sticky end” ligation reaction. A spacer sequence may comprise a fewer number of bases than the encoder sequence within a coding tag.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, from which identifying information about the macromolecule (e.g., UMI information) associated with the recording tag can be trartsferred to the coding tag. Identifying information can comprise any information characterizing a molecule such as information pertaining to sample, fraction, partition, spatial location, interacting neighboring molecule(s), cycle number, etc. Additionally, the presence of UMI information can also be classified as identifying information. In certain embodiments, after a binding agent binds a polypeptide, information from a coding tag linked to a binding agent can be transferred to the recording tag associated with the polypeptide while the binding agent is bound to the polypeptide. In other embodiments, after a binding agent binds a polypeptide, information from a recording tag associated with the polypeptide can be transferred to the coding tag linked to the binding agent while the binding agent is bound to the polypeptide. A recoding tag may be directly linked to a polypeptide, linked to a polypeptide via a multifunctional linker, or associated with a polypeptide by virtue of its proximity (or co-localization) on a solid support. A recording tag may be linked via its 5′ end or 3′ end or at art internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “primer extension”, also referred to as “polymerase extension”, refers to a reaction catalyzed by a nucleic acid polymerase (e.g., DNA polymerase) whereby a nucleic acid molecule oligonucleotide primer, spacer sequence) that anneals to a complementary strand is extended by the polymerase, using the compiementary strand as template.

As used herein, the term “unique molecular identifier” or “UMI” refers to a nucleic acid molecule of about 3 to about 40 bases (3, 4, 5, 6, 7, 8, 9, 10, 11. 12, 13, 14, 15, 16, 17, 18, 19, 20, 21, 27, 23, 24, 25, 26, 27, 28, 29, 30, 31, 32, 33, 34, 35, 36, 37, 38, 39, or 40 bases in length providing a unique identifier tag for each polypeptide or binding agent to which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A binding agent UMI can be used to identify each individual molecular binding agent that binds to a particular polypeptide. For example, a can be used to identify the number of individual binding events for a binding agent specific for a single amino acid that occurs for a particular peptide molecule. It is understood that when UMI and barcode are both referenced in the context of a binding agent or polypeptide, that the barcode refers to identifying information other that the UMI for the individual binding agent or polypeptide (e.g., sample barcode, compartment barcode, binding cycle barcode).

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81). Alternatively, recording tag molecules may be circularized and sequenced directly by polymerase extension from universal priming sites (Korlach et al., 2008, Proc. Natl. Acad. Sci. 105:1176-1184). The term “forward” when used in context with a “universal priming site” or “universal primer” may also be referred to as “5” or “sense”. The term “reverse” when used in context with a “universal priming site” or “universal primer” may also be refetred to as “3′” or “antisense”.

As used herein, the term “extended recording tag” refers to a recording tag to which information of at least one binding agent's coding tag (or its complementary sequence) has been transferred following, binding of the binding agent to a polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise binding agent information of 1, 2, 3, 4, 5, 6, 7, 8, 9, 10, 11, 12, 13, 14, 15, 16, 17, 18, 19, 20, 21, 22, 21, 24, 25, 26, 27, 26, 29, 30, 31, 32, 33, 34, 35, 36, 37, 38, 39, 40, 45, 50, 55, 60, 65, 70, 75, 80, 85, 90, 95, 100, 125, 150, 175, 200 or more coding tags. The base sequence of an extended recording tag may reflect the temporal and sequential order of binding of the binding agents identified by their coding tags, may reflect a partial sequenlial order if binding of the binding agents identifw by the coding tags, or may not reflect any order of binding of the binding agents identified by the coding tags. In certain embodiments, the coding tag information present in the extended recording tag represents with at least 25%, 30%, 35%, 40%, 45%, 50%, 55%, 60%, 65%, 70%, 75%, 80%, 85%, 90%, 91%, 92%, 93%, 94%, 95%, 96%, 97% 98%, 99%, or 100% identity the polypeptide sequence being analyzed. In certain embodiments where the extended recording tag does not represent the polypeptide sequence being analyzed with 100% identity, errors may be due to off-target binding by a binding agent, or to a “missed” binding cycle (e.g., because a binding agent fails to bind to a polypeptide during a binding cycle, because of a failed primer extension reaction), or both.

As used herein, the term “extended coding tag” refers to a coding tag to which information of at least one recording tag (or its complementary sequence) has been transferred following binding of a binding agent, to which the coding tag is joined, to a polypeptide, to which the recording tag is associated. Information off recording tag may be transferred to the coding tag directly (e.g., ligation), or indirectly (e.g., primer extension). Information of a recording tag may be transferred enzymatically or chemically. In certain embodiments, an extended coding tag comprises information of one recording tag, reflecting one binding event. As used herein, the term “di-tag” or “di-tag construct” or “di-tag molecule” refers to a nucleic acid molecule to which information of at least one recording tag (or its complementary sequence) and at least one coding tag (or its complementary sequence) has been transferred following binding of a binding agent, to which the coding tag is joined, to a polypeptide, to which the recording tag is associated (see, e.g., FIG. 11B of International Patent Application Publication No. WO 2017/192633). Information of a recording tag and coding tag may be transferred to the di-tag indirectly (e.g., primer extension). Information of a recording tag may be transferred enzymatically or chemically. In certain embodiments, a di-tag comprises a UMI of a recording tag, a compartment tag of a recording tag, a universal priming site of a recording tag, a UMI of a coding tag, an encoder sequence of a coding tag, a binding cycle specific barcode, a universal priming site of a coding tag, or any combination thereof.

As used herein, the term “solid support”, “solid surface”, “solid substrate”, “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, nylon, a silicon wafer chip, a flow through chip, a flow cell, a biochip including signal transducing electronics, a channel, a microtiter well, an ELISA plate, a spinning interferometry disc, a nitrocellulose membrane, a nitrocelltilose-based polymer surface, a polymer matrix, a nanoparticle, or a microsphere. Materials for a solid support include but are not limited to acrylarnide, agarose, cellulose, nitrocellulose, glass, gold, quartz, polystyrene, polyethylene vinyl acetate, polypropylene, polymethacrylate, polyethylene, polyethylene oxide, poiysilicates, polycarbonates, Teflon, fluorocarbous, nylon, silicon rubber, polyanhydrides, polyglycolic acid, polyactic acid, polyorthoesters, functionalized silane, potypropylfutnerate, collagen, glycosaminoglycans, polynmino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, polystyrene bead, a polymer bead, a methylstyrene bead, an agarose bead, an acrylamide bead, a solid core bead, a porous bead, a paramagnetic bead, a glass bead, or a controlled pore bead. A bead may be spherical or an irregularly shaped. A bead or support may be porous. A bead's size may range from nanometers, e.g., 100 nm, to millimeters, e.g., 1 mm. In certain embodiments, beads range in size from about 0,2 micron to about 200 microns, or from about 0.5 micron to about 5 microns. In some embodiments, beads can be about 1, 1.5, 2, 2,5, 2.8, 3, 3.5, 4, 4,5, 5, 5.5, 6, 6,5, 7, 7.5, 8, 8.5, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, between about 100 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), γPNAs, morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boranophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In same embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, a γPNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymetase to amplify (these groupings are sometimes referred to as polymerase colonies or polonies). Consequently, during the sequencing process, a nucleotide at a particular position can be sequenced multiple times (e.g., hundreds or thousands of times) this depth of coverage is referred to as “deep sequencing.” Examples of high throughput nucleic acid sequencing technology include platforms provided by Illumina, BGI Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays, as reviewed by Service (Science 311:1544-1546, 2006).

As used herein, “single molecule sequencing” or “third generation sequencing” refers to next-generation sequencing methods wherein reads from single molecule sequencing instruments are generated by sequencing of a single molecule of DNA. Unlike next generation sequencing methods that rely on amplification to clone many DNA molecules in parallel for sequencing in a phased approach, single molecule sequencing interrogates single molecules of DNA and does not require amplification or synchronization. Single molecule sequencing includes methods that need to pause the sequencing reaction after each base incorporation (‘wash-and-scan’ cycle) and methods which do not need to halt between read steps. Examples of single molecule sequencing methods include single molecule real-time sequencing (Pacific Biosciences), nanopore-based sequencing (Oxford Nanopore), duplex internmted nanopore sequencing, and direct imaging of DNA using advanced microscopy.

As used herein, “analyzing,” the polypeptide means to quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n-1, n-2, n-3, and so forth). This is accomplished by elimination of the n NTAA, thereby converting the n-1 amino acid of the peptide to an N-terminal amino acid (referred to herein as the “n-1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

As used herein, the term “compartment” refers to a physical area or volume that separates or isolates a subset ofpolypeptides from a sample of polypeptides. For example, a compartment may separate an individual cell from other cells, or a subset of a sainples proteome from the rest of the sample's proteome. A compartment may be an aqueous compartment (e.g., microfluidic droplet) a solid compartment (e.g., picotiter well or microtiter well on a plate, tube, vial, gel bead), a bead surface, a pomis bead interior or a separated region on a surface. A compartment may comprise one or more beads to which polypeptides may be immobilized.

As used herein, the term “compartment tag” or “compartment barcode” refers to a single or double stranded nucleic acid molecule of about 4 bases to about 100 bases (including 4 bases, 100 bases, and any integer between) that comprises identifying information for the constituents (e.g a single cell's proteome), within one or more compartments (e.g., microfluidic droplet or bead surface, etc.). A compartment barcode identifies a subset of polypeptides in a sample that have been separated into the same physical compartment or group of compartments from a plurality (e.g., millions to billions) of compartments. Thus, a compartment tag can be used to distinguish constituents derived from one or more compattinents having the same compartment tag from those in another compartment havinh a different compartment tag, even after the constituents are pooled together. By labeling the proteins and/or peptides within each compartment or within a group of two or more compartments with a unique compartment tag, peptides derived from the same protein, protein complex, or cell within an individual compartment or group of compartments can be identified. A compartment tag comprises a barcode, which is optionally flanked by a spacer sequence on one or both sides, and an optional universal primer. The spacer sequence can be complementary to the spacer sequence of a recording tag, enabling transfer of comparMient tag information to the recording tag. A compartment tag may also comprise a universal priming site, a unique molecular identifier (for providing identifying information for the peptide attached thereto), or both, particularly for embodiments where a compartment tag comprises a recording tag to be used in downstream peptide analysis methods described herein. A compartment tag can comprise a functional moiety (e.g., aldehyde, NHS, mTet, alkyne, etc.) for coupling to a peptide. Alternatively, a compartment tag can comprise a peptide comprising a recognition sequence for a protein ligase to allow ligation of the compartment tag to a peptide of interest. A compartment can comprise a single compartment tag, a plurality of identical compartment tags save for an optional UMI sequence, or two or more different compartment tags. In certain embodiments each compartment comprises a unique compartment tag (one-to-one mapping). In other embodiments, multiple compartments from a larger population of compartments comprise the same compartment tag (many-to-one mapping). A compartment tag may be joined to a solid support within a compartment (e.g., bead) or joined to the surface of the compartment itself (e.g., surface of a picotiter well). Alternatively, a compartment tag may be free in solution within a compartment.

As used herein, the term “partition” refers to an assignment, e.g., a random assignment, of a unique barcode to a subpopulation of polypeptides from a population of polypeptides within a sample. In certain embodiments, partitioning may be achieved by distributing polypeptides into compartments. A partition may be comprised of the polypeptides within a single compartment or the polypeptides within multiple compartments from a population of compartments.

As used herein, a “partition tag” or “partition barcode” refers to a single or double stranded nucleic acid molecule of about 4 bases to about 100 bases (including 4 bases, 100 bases, and any integer between) that comprises identifying information tor a partition. In certain embodiments, a partition tag for a polypeptide refers to identical compartment tags arising from the partitioning of polypeptides into compartments) labeled with the same barcode.

As used herein, the term “fraction” refers to a subset of polypeptides within a sample that have been sorted from the rest of the sample or organelles using physical or chemical separation methods, such as fractionating by size, hydrophobicity, isoelectric point, affinity, and so on. Separation methods include HPLC separation, gel separation, affinity separation, cellular fractionation, cellular organelle fractionation, tissue fractionation, etc. Physical properties such as fluid flow, magnetism, electrical current, mass, density, or the like can also be used for separation.

As used herein, the term “fraction barcode” refers to a single or double stranded nucleic acid molecule of about 4 bases to about 100 bases (including 4 bases, 100 bases, and any integer therebetween) that comprises identifying information for the polypeptides within a fraction

### I. Methods of Proximity Interaction Analysis via Forming a Linking Structure

In one aspect, tlie present disclosure provides a method for assessing identity and spatial relationship between a polypeptide and a moiety in a sample, which method comprises: a) forming a linking structure between a site of i polypeptide in a sample and a site of a moiety in said sample, said linking structure comprising a polypeptide tag associated with said site of said polypeptide and a moiety tag associated with said site of said moiety, wherein said polypeptide tag and said moiety tag are associated; b) transferring information between said associated polypeptide tag and said moiety tag cr ligating said associated polypeptide tag and said moiety tag to form a shared unique molecule identifier (UMI) and/or barcode; c) breaking said linking structure via dissociating said polypeptide from said moiety and dissociating said polypeptide tag from said moiety tag, while maintaining association between said polypeptide atnl said polypeptide tag. and maintaining associttion between said moiety and said moiety tag; and d) assessing said polypeptide tag and at least a partial sequence of said polypeptide, and assessing said moiety tag and at least a partial identity of said moiety, wherein said assessed portions of said polypeptide tag and said moiety tag comprise said shared unique molecule identifier (UMI) and/or barcode indicates that said site of said polypeptide and said site of said moiety in said sample are in spatial proximity.

Also provided herein is a method for assessing identity and spatial relationship between a polypeptide and a moiety in a sample including, a) forming a linking structure between a site of a polypeptide in a sample and a site of a moiety in said sample, said linking structure comprising a polypeptide tag associated with said site of said polypeptide and a moiety tag associated with said she of said moiety, wherein said polypeptide tag and said moiety tag are associated; b) transferring information between said associated polypeptide tag and said moiety tag to form a shared unique molecule identifier (UMI) and/or barcode, wherein the shared UMI and/or barcode is formed as a separate record polynucleotide; c) breaking said linking structure via dissociating said polypeptide from said moiety and dissociating said polypeptide tag from said moiety tag, while maintaining association between said polypeptide and said polypeptide tag, and maintaining association between said moiety and said moiety tag; d) assessing said polypeptide tag and at least a partial sequence of said polypeptide, and assessing said moiety tag and at least a partial identity of said moiety; and e) assessing said separate record polynucleotide to establish the spatial relationship between the site of the polypeptide and the site of the moiety. In some embodiments, step e) establishes the spatial relationship between the site of the polypeptide and two or more sites of said moiety or two or more moieties. In some embodiments, the separate record polynucleotide is released from said polypeptide tag and/or said moiety tag.

Any suitable moiety can be used in the present methods. For example, the moiety can be an atom, an inorganic moiety, an organic moiety or a complex thereof. The organic moiety can be an amino acid, a polypeptide, e.g., a peptide or a protein, a nucleoside, a nucleotide, a polynucleotide, e.g., an oligonucleotide or a nucleic acid, a vitamin, a monosaccharide, an oligosaecharide, a carbohydrate, a lipid and a complex thereof. In, some embodiments, the moiety can comprise a polypeptide. In other embodiments, the moiety can comprise a polynucleotide.

In some embodiments, the poly peptide and/or moiety has a three-dimensional structure. In some embodiments, the polypeptide and the moiety belong to different molecules, and the present methods can be used to assess identity and spatial relationship between the polypeptide and the moiety in different molecules, e.g., in a protein-protein complex, a protein-DNA complex or a protein-RNA complex. A macromolecule assembly may be composed of the same type of macromolecule (e.g., protein-protein) or of two or more different types of macromolecules (e.g., protein-DNA). In other embodiments, the polypeptide and the moiety belong to the same macromolecule.

A. Exemplary Tags and Components

Any suitable polypeptide tag can be used in the present methods. For example, the polypeptide tag can be an atom, an inorganic moiety, an organic moiety or a complex thereof. The organic moiety can be an amino acid, a polypeptide, e.g., a peptide or a protein, a nucleoside, a nucleotide, a polynucleotide, e.g., ari oligonucleotide or a nucleic acid, a vitamin, a monosaccharide, an oligosaccharide, a carbohydrate, a lipid and a complex thereof. In some embodiments, the polypeptide tag can comprise a polynucleotide.

Any suitable moiety tag can be used in the present methods. For example, the moiety tag can be an atom, an inorganic moiety, an organic moiety or a complex thereof. The organic moiety can be an amino acid, a polypeptide, e.g., a peptide or a protein, a nucleoside, a nucleotide, a polynucleotide, eg., are oligonudeotide or a nucleic acid, a vitamin, a monosaccharide, an oligosaccharide, a carbohydrate, a lipid and a complex thereof. In some embodiments, the moiety tag can comprise a polynucleotide.

Both the polypeptide tag and the moiety tag can comprise polynucleotides. In some embodiments, the polypeptide tag comprises a UMI and/or barcode. In some embodiments, the moiety tag comprises a UMI and/or barcode. In some embodiments, the polypeptide tag comprises a first polynucleotide and the moiety tag comprises a second polyaucleotide, the first and second polynucleotides comprise a complementary sequence, and the polypeptide tag and the moiety tag are associated via the complementary sequence, in some embodiments, the sequence and complementary sequence comprise a palindromic sequence, in some embodiments, the polypeptide tag and/or moiety tag does not comprise a palindromic sequence.

In some embodiments, the polypeptide tag and the moiety tag are used for creating a separate record polynucleotide. In some embodiments, the separate record polynucleotide is or comprises a DNA or RNA molecule. In some embodiments, the separate record polynucleotide comprises information regarding one or more polypeptides and/or one or more moieties.

In some embodiments, the polypeptide tag and the separate record poly meleotide comprises a complementary sequence. In some embodiments, the polypeptide tag and the separate record polynucleotide are associated via the complementary sequence. In some embodiments, the moiety tag and the separate record polynucleotide comprise a complementary sequence. In some cases, the moiety tag arid the separate record polynucleotide are associated via the complementary sequence.

In some embodiments, the polypeptide tag and the moiety tag each comprises one or more nucleic acid strand(s) arranged into a double-stranded palindromic region, a double stranded barcode region, and/or a primer binding region. In some cases, the polypeptide tag and the moiety tag comprise the following in the order listed: palindromic region—barcode region—primer-binding region. In some embodiments, the polypeptide tag and the moiety tag each comprise a hairpin structure baying a partially-double-stranded primer-binding region, a double-stranded barcode region, a double-stranded palindromic region, and a single-stranded loop region containing a target-binding moiety. In some embodiments, a molecule that terminates polymerization is located between the double-stranded palindromic region and the loop region.

In some embodiments, the moiety tag and/or the polypeptide tag comprise one or more nucleic acid strands arranged into a double-stranded palindromic region, a double-stranded barcode region, and/or a primer-binding region. In some embodiments, the tags are arranged to form a hairpin structure, which is a single stretch of contiguous nucleotides that folds and forms a double-stranded region, referred to as a “stem,” and a single-stranded region, referred to as a “loop.” The double-stranded region is formed when nucleotides of two regions of the same nucleic acid base pair with each other (intramolecular base pairing).

In some embodiments, the polypeptide tag and/or the moiety tag comprise a two parallel nucleic acid strands (e.g., as two separate nucleic acids or as a contiguous folded hairpin). One of the strands is referred to as a “complementary strand,” and the other strand is referred to as a “displacement strand.” The complementary strand typically contains the primer-binding region, or at least a single-stranded segment of the primer-binding region, where the primer binds (e.g., hybridizes). The complementary strand and the displacement strand are bound to each other at least through a double-stranded barcoded region and through a double-stranded palindromic region. The “displacemest strand” is the strand that is initially displaced by a newly-generated half-record, as described herein, and, in turn, displaces the newly-generated half-record as the displacement strard “re-binds” to the complementary strand.

Two nucleic acids or two nucleic acid regions are “complementary” to one another if they basc-pair, or bind, to each other to form a double-stranded nucleic acid molecule via Watson-Crick interactions (also referred to as hybridization). As used herein, “binding” refers to an association between at least two molecules due to, for example, electrostatic, hydrophobic, ionic and/or hydrogen-bond interactions under physiological conditions.

A “double-stranded region” of a nucleic acid refers to a region of a nucleic acid (e.g., DNA or RNA) containing two parallel nucleic acid strands bound to each other by hydrogen bonds between complementary purines (e.g., adenine and guanine) and pyrimidines (e.g., thymine, cytosine and uracil), thereby forming a double helix. In some embodiments, the two parallel nucleic acid strands forming the double-stranded region are part of a contiguous nucleic acid strand. For example, the polypeptide tag and moiety tag can comprise a hairpin structure or are attached to a hairpin structure.

A “double-stranded palindromic region” refers to a region of a nucleic acid (e.g., DNA or RNA) that is the same sequence of nucleotides whether read 5′ (five-prime) to 3′ (three prime) on one strand or 5′ to 3′ on the complementary strand with which it forms a double helix.

In some embodiments, palindromic sequences permit joining of the polypeptide tag and moiety tag that are proximate to each other. Polymerase extension of a primer bound to the primer-binding region produces a “half-record,” which refers to the newly generated nucleic acid strand. Generation of the half record displaces one of the strands of the polypeptide or moiety tag, referred to as the “displacement strand.” This displacement strand, in turn, displaces a portion of the half record (by binding to its “complementary strand”), starting at the 3′ end, enabling the 3′ end of the half record, containing the palindromic sequence, to bind to another half record similarly displaced from a proximate barcoded nucleic acid.

In some embodiments, a double-stranded palindromic region has a length of 4 to 10 nucleotide base pairs. That is, in some embodiments, a double-stranded palindromic region may comprise 4 to 10 contiguous nucleotides bound to 4 to 10 respectively complementary nucleotides. For example, a double-stranded palindromic region may have a length of 4, 5, 6, 7, 8, 9 or 10 nucleotide base pairs. In some embodiments, a double-stranded palindromic region may have a length of 5 to 6 nucleotide base pairs. In some embodiments, the doable-stranded palindromic region is longer than 10 nucleotide base pairs. For example, the double-stranded palindromic region may have a length of 4 to 50 nucleotide base pairs. In some embodiments, the double-stranded palindromic region has a length of 4 to 40, 4 to 30, or 4 to 20 nucleotide base pairs.

A double-stranded palindromic region may comprise guanine (G), cytosine (C), adenine (A) and/or thymine (T). In some embodiments, the percentage of G and C nucleotide base pairs (G/C) relative to A and T nucleotide base pairs (A/T) is greater than 50%. For example, the percentage of G/C, relative to A/T of a double-stranded palindromic region may be 50% to 100%. In some embodiments, the percentage of G/C relative to A/T is greater than 60%, 65%, 70%, 75%, 80%, 85%, 90%, 95%.

In some embodiments, a double-stranded palindromic region may include an even number of nucleotide base pairs, although double-stranded palindromic region of the present disclosure are not so limited. For example, a double-stranded palindromic region may include 4, 6, 8 or 10 nucleotide base pairs. Alternatively, a double-stranded palindromic region may include 5, 7 or 9 nucleotide base pairs.

Among a plurality of polypeptide and moiety tags, typically, the ouble-stranded palindromic regions are the same for each tag of the plurality such that a polypeptide tag proximate to a moiety tag are able to bind to each other through generated half-records containing the palindromic sequence. In some embodiments, however, the double-stranded palindromic regions may be the same only among a subset of polypeptidelmoiety tags such that two different subsets contain two different double-stranded palindromic regions.

A “primer-binding region” refers to a region of a nucleic acid (e.g., DNA or RNA) comprising the moiety tag or polypeptide tag where a single-stranded primer (e.g., DNA or RNA primer) binds to start replication. A primer-binding region may be a single stranded region or a partially double stranded region, which refers to a region containing both a single-stranded segment and a double-stranded segment. A primer-binding region may comprise any combination of nucleotides in random or rationally-designed order. In some embodiments, a primer-binding region has a length of 4 to 40 nucleotides (or nucleotide base pairs, or a combination of nucleotides and nucleotide base pairs, depending the single- and/or double-stranded nature of the primer-binding region). For example, a primer-binding region may have a length of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides (and/or nucleotide base pairs). In some embodiments, a primer-binding region may have a length of 4 to 10, 4 to 15, 4 to 20, 4 to 25, 4 to 30, 4 to 35, or 4 to 40 nucleotides (and/or nucleotide base pairs). In some embodiments, a primer-binding region is longer than 40 nucleotides. For example, a primer-binding region may have a length of 4 to 100 nucleotides. In some embodiments, a primer-binding region has a length of 4 to 90, 4 to 80, 4 to 70, 4 to 60, or 4 to 50 nucleotides.

In some embodiments, a primer-binding region is designed to accommodate binding of more than one (e.g., 2 or 3 different) primers. A “primer” is a single-stranded nucleic acid that serves as a starting point for nucleic acid synthesis. A polymerase adds nucleotides to a primer to generate a new nucleic acid strand. Primers of the present disclosure are designed to be complementary to and to bind to the primer-binding region of the polypeptide tag or the moiety tag. Thus, primer length and composition (e.g., nucleotide composition) depend, at least in part, on the length and composition of a primer-binding region of a polypeptide or moiety tag. In some embodiments, a primer has a length of 4 to 40 nucleotides. For example, a primer may have a length of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. In some embodiments, a primer may have a lerwth of 4 to 10, 4 to 15, 4 to 20, 4 to 25, 4 to 30, 4 to 35, or 4 to 40 nucleotides.

Primers may exist attached in pairs or other combinations (e.g., triplets or more, in any geometry) for the purpose, for example, of restricting binding to those meeting their geometric criteria. The rigid, double-stranded linkage shown enforces both a minimum and a maximum distance between a moiety tag and polypeptide tag. The double-stranded “ruler” domain may be any length (e.g., 2 to 100 nucleotides, or more) and may optionally include a barcode itself that links the two halves by information content, should they become separated during processing. In some embodiments, a double stranded ruler domain, which enforces a typical distance between a moiety tag and polypeptide tag at which records may be generated, is a complex structure, such as a 2-, 3-, or 4-DNA helix bundle, DNA nanostructure, such as a DNA origami structure, or other structure that adds or modifies the stifthess/rigidity of the ruler.

A “strand-displacing polymerase” refers to a polymerase that is capable of displacing downstream nucleic acid (e.g., DNA) encountered during nucleic acid synthesis. Different polymerases can have varying degrees of displacement activity. Examples of strand-displacing polymerases include, without limitation, list large fragment polymerase (e.g., New England Biolabs (NEB) #M0275), phi 29 polymerase (e.g., NEB #M0269), Deep VentR polymerase, Klenow fragment polymerase, and modified Tag polymerase. Other strand-displacing polymerases are contemplated.

In some embodiments, a primer comprises at least one nucleotide mismatch relative to the single-stranded primer-binding region. Such a mismatch may be used facilitate displacement of a half record from the complementary strand of the moiety tag and/or polypeptide tag. In some embodiments, a primer comprises at least one artificial linker.

In some embodiments, extension of a primer (bound to a primer-binding site) by a displacing polymerase is typically terminated by the presence of a molecule or modification that terminates polymerization. Thus, in some embodiments, the moiety tag and/or polypeptide tag may comprise a molecule or modification that terminates polymerization. A molecule or modification that terminates polymerization (“stopper” or “blocker”) is typically located in a double-stranded region of the moiety tag or polypeptide tag, adjacent to tlie double-stranded palindromic region, such that polymerization terminates extension of the primer through the double-stranded palindromic region. For moiety or polypeptide tags arranged in the form of a hairpin, a molecule or modification that terminates polymerization may be located between the double-stranded palindromic region and the hairpin loop. In some embodiments, the molecule that terminates polymerization is a synthetic non-DNA linker, for example, a triethylene glycol spacer, such as the Int Spacer 9 (iSp9), C3 Spacer, or Spacer 18 (Integrated DNA Technologies (IDT). It should be understood that any non-native linker that terminates polymerization by a polymerase may be used as provided herein. Other non-limiting examples of such molecules and modifications include a three-carbon linkage (/iSpC3/) (IDT), ACRYDITE™ (IDT), adenylation, azide, digoxigenin (NHS ester), cholesteryl-TEG (IDT), I-LINKER™ (IDT), and 3-cyanovinylcarbazole (CNVK) and variants thereof. Typically, but not always, short linkers (e.g., iSp9) lead to faster reaction times.

In some embodiments, the molecule that terminates polymerization is a single or paired non-natural nucleotide sequence, such as iso-dG and iso-dC (IDT), which are chemical variants of cytosine and guanine, respectively. Iso-dC will base pair (hydrogen bond) with Iso-dG but not with dG. Similarly, Iso-dG will base pair with Iso-dC but not with dC. By incorporating these nucleotides in a pair on opposite sides of the hairpin, at the stopper position, tltc polymerase will be halted, as it does not have a complementary nucleotide in solution to add at that position.

In some embodiments, the efficiency of performance of a “stopper” or “blocker” modification be improved by lowering dNTP concentrations (e.g., from 200 μm) in a reaction to 100 μm, 10 μm, 1 μm, or less.

Inclusion of a molecule or modificstion that terminates polymerization often creates a “bulge” in a double-stranded region of the moiety tag or polypeptide tag (eg., a stem region for hairpin structures) because the molecule or modification is not paired. Thus, in some embodiments, the moiety and/or polypeptide tags are designed to include, opposite the molecule or modification, a single nucleotide (e.g., thymine), at least two of same nucleotide (e.g., a thymine dimer (TT) or trimer (TTT)), or an non-natural modification.

In some aspects, to prevent the polymerase from extending an end (e.g., a 5′ or 3′ end) of a moiety tag and/or polypeptide tag, a poly-T sequence (e.g., a sequence of 2, 3, 4, 5, 7, 8, 9 or 10 thymine nucleotides) may be used. Alternatively, a synthetic base (e.g., an inverted dT) or other modification may be added to an end (e.g., a 5′ or 3′ end) of the tag to prevent unwanted polymerization of the tag. Other termination molecules (molecules that prevent extension of a 3′ end not intended to be extended) include, without limitation, iso-dG and iso-dC or other unnatural nucleotides or modifications.

In some embodiments, generation of a half record displaces one of the strands of the moiety tag or polypeptide tag. This displaced strand, in turn, displaces a portion of the half record, starting at the 3′ end. This displacement of the half-record is facilitated, in some embodiments, by a “double-stranded displacement region” adjacent to the molecule or modification that terminates polymerization. In embodiments wherein the moiety tag and/or polypeptide tag has a hairpin stincture, the double-stranded displacement region may be located between the molecule or modification that terminates polymerization and the hairpin loop. A double-stranded displacement region may comprise any combination of nucleotides in random or rationally-designed order. In some embodiments, a double-stranded displacement region has a length of 2 to 10 nucleotide base pairs. For example, a double-stranded displacement region may have a length of 2, 3, 4, 5, 6, 7, 8, 9 or 10 nucleotide base pairs. In some embodiments, a double-stranded palindromic region may have alength of 5 to 6 nucleotide base pairs. In some embodiments, a double-stranded palindromic region may contain only a combination of C and G nucleotides.

Displacement of the half-record may also be facilitated, in some embodiments, by modifying the reaction conditions. For example, some auto-cyclic reactions may include, instead of natural, soluble dNTPs for new strand generation, phosphorothioate nucleotides (2′-Deoxynucleoside Alpha-Thiol 2′-Deoxynucleoside Alpha-Thiol Triphosphate Set, Trilink Biotechnologies). These are less stable in hybridization that natural dNTPs, and result in a weakened interaction between half record and stem. They may be used in any combination (e.g., phosphorothioate A with natural T, C, and G bases, or other combinations or ratios of mixtures). Other such chemical modifications may be made to weaken the half record pairing and facilitate displacement.

In some embodiments, the moiety tag and/or polypeptide tag itself may be modified, in some embodiments, with unnatural nucleotides that serve instead to strengthen the hairpin stem. In such embodiments, the displacing polymerase that geneintes the half record can still open and copy the stem, but, during strand displacement, stem sequence re-hybridization is energetically favorable over half-record hybridization with stem template. Non-limiting examples of unnatural nucleotides include 5-methyl dC (5-methyl deoxycytidine; when substituted for dC, this molecule increase the melting temperature of nucleic acid by as much as 5° C. per nucleotide insertion), 2,6-diaminopurine (this molecule can increase the melting temperature by as much as 1-2° C. per insertion), Super T (5-hydroxybutynl-2′-deoxyuridine also increases melting temperature of nucleic acid), and/or locked nucleic acids (LNAs). They may occur in either or both strands of the hairpin stem.

In some embodiments, unnatural nucleotides may be used to introduce mismatches between new half record sequence and the stem. For example, if an isoG nucleotide existed in the template strand of the stem, a polymerase, in some cases, will mistakenly add one of the soluble nucleotides available to extend the half record, and in doing so create a ‘bulge’ between the new half record and the stem template strand, much like the bulge (included in the primer). It will, in some aspects, serve the same purpose of weakening half-record-template interaction and encourage displacement.

In some embodiments, the molety tag and/or the polypeptide tag are arranged to form a hairpin structure, which is a single stretch of contiguous nucleotides that folds and forms a double-stranded region, referred to as a “stem,” and a single-stranded region, referred to as a “loop.” In some embodiments, the single-stranded loop region has a length of 3 to 50 nucleotides. For example, the single-stranded loop region may have a length of 3, 4, 5, 6, 7, 8, 9 or 10 nucleotides. In some embodiments, the single-stranded loop region has a length of 3 to 10, 3 to 15, 3 to 20, 3 to 25, 3 to 30, 3 to 35, 3 to 40, 3 to 45, or 3 to 50 nucleotides. In some embodiments, the single-stranded loop region is longer than 50 nucleotides. For example, the single-stranded loop region may have a length of 3 to 200 nucleotides. In some embodiments, the single-stranded loop region has a length of 3 to 175, 3 to 150, 3 to 100, or 3 to 75 nucleotides. In some embodiments, a loop region includes smaller regions of intramolecular base pairing. A hairpin loop, in some embodiments permits flexibility in the orientation of the moiety tag and/or the polypeptide tag relative to a target binding-moiety. That is, the loop typically allows the moiety tag or the polypeptide tag to occupy a variety of positions and angles with respect to the target-binding moiety, thereby permitting interactions with a multitude of nearby tags (e.g., attached to other targets) in succession.

The moiety tag and/or the polypeptide tag, in some embodiments, comprise at least one locked nucleic acid (LNA) nucleotides or other modified base. Pairs of LNAs, or other modified bases, can serve as stronger (or weaker) base pairs in double-stranded regions of the moiety tag and/or the polypeptide tag, thus biasing the strand displacement reaction. In some embodiments, at least one LNA molecule is located on a complementary stranded of a tag, between a double-strand.ed barcoded region and a single-stranded primer-binding region.

The moiety tag and/or the polypeptide tag may be DNA such as D-form DNA and L-form DNA and RNA, as well as various modifications thereof. Nucleic acid modifications include base modifications, sugar modifications, and backbone modifications. Non-limiting examples of such imidifications are provided below.

Examples of modified nucleic acids (e.g., DNA variants) that may be used in accordance with the present disclosure include, without limitation, L-DNA (the backbone enantiomer of DNA, known in the literature), peptide nucleic acids (PNA) bisPNA clamp, a psendocomplementary PNA, locked nucleic acid (LNA), and co-nucleic acids of the above such as DNA-LNA co-nucleic acids. Thus, the present disclosure contemplates nanostructures that comprise DNA, RNA, LNA, PNA or combinations thereof. It is to be understood that the nucleic acids used in methods and compositions of the present disclosure may be homogeneous or heterogeneous in nature. As an example, nucleic acids may be completely DNA in nature or they may be comprised of DNA and non-DNA (e.g., LNA) monomers or sequences. Thus, any combination of nucleic acid elements may be used. The nucleic acid modification may render the nucleic acid more stable and/or less susceptible to degradation under certain conditions. For example, in some embodiments, nucleic acids are nuclease-resistant.

Also provided herein are pluralities of moiety tags and the polypeptide tags. A “plurality” comprises at least two tags. In some embodiments, a plurality comprises 2 to 2 million tags (e.g., unique tags). For example, a plurality may comprise 100, 500, 1000, 5000, 10000, 100000, 1000000, or more, tags. This present disclosure is not limited in this aspect.

B. Information Transfer

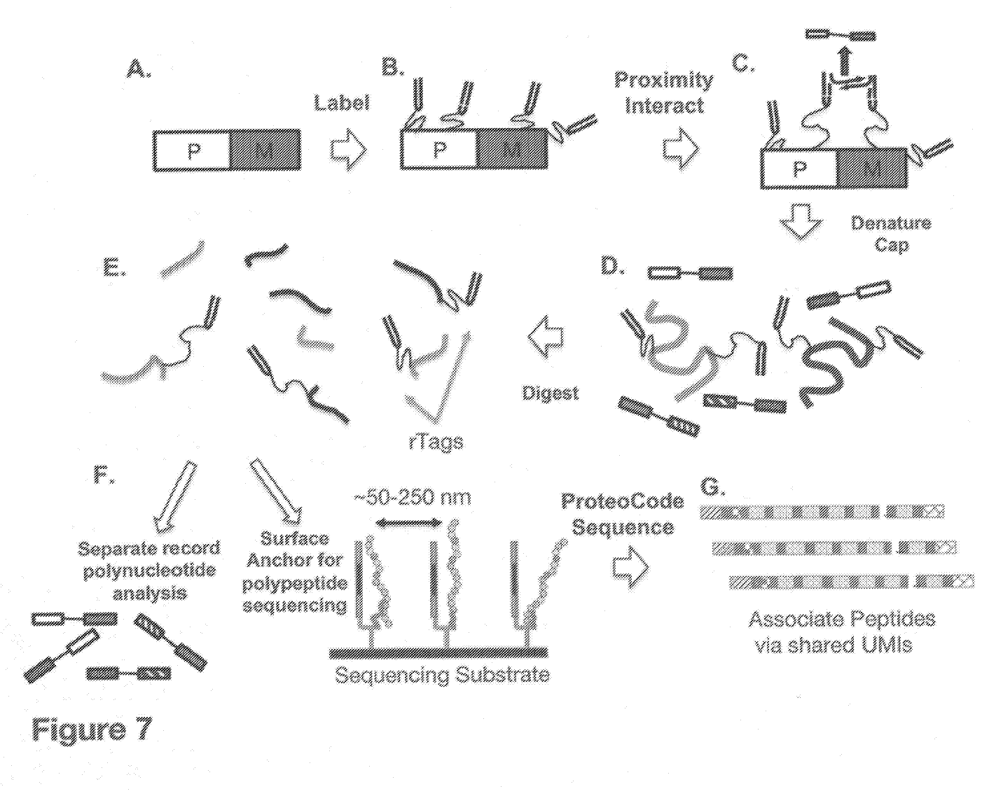

Information between the associated polypeptide tag and moiety tag can be transferred in any suitable manner to form the shared UMI and/or barcode. In some embodiments, information between the associated polypeptide tag and moiety tag can be transferred to a separate record polynucleotide (e.g., FIG. 7C). In some embodiments, the separate record polynucleotide is a newly fowled polypeptide that comprises the shared UMI and/or barcode.

In some embodiments, transferring information between the associated polypeptide tag and moiety tag comprises extending both the first polynucleotide of the polypeptide tag and the second polynucleotide of the moiety tag to form the shared UMI and/or barcode. In other embodiments, transferring information between the associated polypeptide tag and moiety tag comprises extending one of the first polynucleotide of the polypeptide tag and the second polynucleotide of the moiety tag to form the shared UMI and/or barcode. In still other embodiments, the polypeptide tag comprises a double-stranded polyancleotide and the moiety tag comprise a double-stranded polynucleotide, and transferring information between the associated polypeptide tag and moiety tag comprises ligating the double-stranded polynueleotides to form the shared UMI and/or barcode. The shared UMI and/or barcode can comprise sequences of both the double-stranded polynucleotides. The shared UMI and/or barcode can also comprise sequence of one of the double-stranded polynucleotides. In some embodiments, transferring information between the associated polypeptide tag and moiety tag comprises extending the poly-peptide tag and the moiety tag followed by a ligation reaction to form a double-stranded separate record polynucleotide comprising information from the polypeptide tag and the moiety tag (e.g., shared UMI and/or barcode).

In some embodiments, the shared unique molecule identifier (UMI) and/or barcode comprises information regarding one or more polypeptides and/or one or more moieties.

In some embodiments, information transfer between the associated polypeptide tag and moiety tag can be mediated by a polymerase, e.g., a DNA polymerase, an RNA polyrnerase, or a reverse transcriptase. In other embodiments, information transfer between the associated polypeptide tag and moiety tag can be mediated by a ligase, e.g., a DNA ligase, a ssDNA ligase (e.g., Circligase), a dsDNA ligase, or an RNA ligase. In other embodiments, information transfer between the associated polypeptide tag and the moiety tag can be mediated by a topoisomerase. In other embodiments, information transfer between the associated polypeptide tag and moiety tag can be mediated by chemical ligation. In some embodiments, information transfer between the associated polypeptide tag and moiety tag can be mediated by extension and/or ligation.

In the linking structure, the polypeptide tag and the moiety tag can be associated in any suitable manner. In some embodiments, the linking structure between the polypeptide tag and the moiety tag and their respective polypeptide and moiety can be joined using methods of covalent cross-linking as described by Schenider et al. and Holding in cross-linking mass spectrometry for proteoirlic applications (Holding 2015, Schneider, Belsorn et al. 2018). In some embodiments, in the linking structure, the polypeptide tag and the moiety tag can be associated stably or covalently. In other embodiments, in the linking structure, the polypeptide tag and the moiety tag can be associated transiently. The association between the polypeptide tag, and the moiety tag can vary over time or over performance of the present methods. The association between the polypeptide tag and the moiety tag can be different before and after information transfer between the polypeptide tag and the moiety tag. For example, in the linking structure, the polypeptide tag and the moiety tag can be associated transiently before the information transfer between the polypeptide tag and the moiety tag. After the information transfer between the polypeptide tag and the moiety tag, the association between the polypeptide tag and the moiety tag can become more stabilized. In still other embodiments, in the linking structure, the polypeptide tag and the moiety tag can be associated directly. In yet other embodiments, in the linking structure, the polypeptide tag and the moiety tag can be associated indirectly, e.g., via a linker or UMI between the polypeptide tag and the moiety tag.

In some of any of the provided embodiments, in the linking structure, the polypeptide tag and the separate record polynucieotide are associated directly. In some of any of the provided embodiments, in the linking structure, the moiety tag and the separate record polynucleotide are associated directly. In some embodiments, in the linking structure, the polypeptide tag and the moiety tag can be associated via a separate record polynucleotide. In some embodiments, the linking structure formed between the polypeptide tag and the moiety tag via the separate record polynucleotide is transient. In some embodiments, the separate record polynucleotide is formed by extension between the polypeptide tag and the moiety tag. In some embodiments, the separate record polynueleotide comprises complementary sequences to the polypeptide tag and the moiety tag. In some embodiments, the separate record polynucleotide is formed by ligation. For example, in some embodiments, the separate record polynucleotide is formed by ligation of the polypeptide tag and the moiety tag.

In forming the linking structure, any suitable number of the polypeptide tag(s) can be associated with a suitable number of site(s) of the polypeptide. For example, in forming the linking structure, a single polypeptide tag can be associated with a single site of the polypeptide, a single polypeptide tag can be associated with a plurality of sites of the polypeptide, or a plurality of the polypeptide tags can be associated with a plurality of sites of the polypeptide. Similarly, in forming the linking structure, any suitable number of the moiety tag(s) can be associated with a suitable number of site(s) of the moiety. For example, in forming the linking structure, a single moiety tag can be associated with a single site of the moiety, a single moiety tag can be associated with a plurality of sites of the moiety, or a plurality of the moiety tags can be associated with a plurality of sites of the moiety.

In some embodiments, information transfer between the associated polypeptide tag and moiety tag to the separate record polynucleotide uses cyclic annealing, extension, and ligation. For example, in some eases, the polypeptide tag, and moiety tag is used as a template to generate double stranded DNA tags (e.g., using primer extension). In some embodiments, the double stranded DNA tags (e.g., polypeptide tag and moiety tag) are ligated. In some embodiments, the DNA tag is or comprises a separate record polynueleotide. In some embodiments, the separate record polynucleotides are thrther PCR amplified.

In some embodiments, information transfer between the associated polypeptide tag and moiety tag to the separate record polynucleotide can be mediated by a polymerase, e.g., a DNA polymerase, an RNA polymerase, or a reverse transcriptase. In some embodiments, the transfer is based on an “autocycle” reaction (See e.g., Schaus et al., Nat Comm (2017) 8:696; and U.S. Patent Application Publication No. US 2018/0010174 and International Patent Application Publication No. WO 2018/017914 and WO 2017/143006). In some embodiments of the repetitive autocycling which forms separate record polynucleotides, the reaction takes place at or around 37° C. in the presence of a displacing polymerase. The polypeptide tag and moiety tag associated with the polypeptide and moiety respectively are bareoded, and are designed such that in the presence of a displacing polymerase and a universal, soluble primer, the moiety tag and/or the polypeptide tag direct an auto-cyclic process that repeatedly produces records of proximate tags. In some specific embodiments, the auto-cyclic process for transferring information includes 1) applying pairs of primer exchange hairpins as a polypeptide or moiety tag, with individual extension to bound half records, 2) strand displacement and 3′ palindromic domain hybridization, and 3) half-record extension to a separate record polynucleotide.

In some further embodiments, the method includes, in a first step, a soluble universal primer binds each of the polypeptide tag and the moiety tag ata common single-stranded primer-binding region, and a displacing polymerase extends the primer through the barcode region and a palindromic region to a molecule or modification that terminates polymerization (e.g., a synthetic non-DNA linker), thereby generating a “half-record,” which refers to a newly generated nucleic acid stand. Secondly, the half records are partially displaced from the barcoded polypeptide or moiety tag by a “strand displacement” mechanism (see, e.g., Yurke et al., Nature 406: 605-608, 2000; and Zhang et al. Nature Chemistry 3: 103-113, 2011, each of which is incorporated by reference herein), and proximate half-records hybridize to each other through the 3′ palindromic regions. Thirdly, the half-records are extended through the barcode regions and primer-binding regions, releasing soluble, separate record polynucleotides that include information from both polypeptide tag and the moiety tag. The polypeptide tag and moiety tag associated with the same or other molecular pairings (other polypeptide-moiety parings or interactions) undergo similar cycling to form separate record polynucleotides.

In some embodiments, upon termination of the cycling reaction, separate record polynucleotides are collected, prepared, amplified, analyzed and/or sequenced (e.g., using parallel next generation sequencing techniques). In some embodiments, the separate record polynucleotides are sequenced, thereby producing sequencing data. In some embodiments, separate record polynucleotides are collected and modified. In some embodiments, separate record polynucleotides are collected and attached (e.g., concatenated). In some embodiments, the method comprises concatenating said collected separate record polynucleotides prior to assessing said separate record polynucleotide. For example, in scririe embodiments, the concatenating is mediated by a ligase or by Gibson assembly. In some embodiments, the concatenated separate record polynucleotides are analyzed, assessed, or sequenced using any suitable techniques, r procedures. For example, the concatenated separate record polynucleotides are sequenced as a suing. In some embodiments, the concatenated polynucleotide is sequenced using nanopore sequencing.

In some embodiments, the, separate record polynucleotides are assessed, and the assessing of the shared unique molecule identifier (UMI) and/or barcode indicates that the site of the polypeptide and said site of the moiety are in spatial proximity. In some embodiments, the sequence data represents spatial configurations and, in some instances, connectivities and/or interactions, of the macromolecules. In some embodiments, the method further includes reconstruction and/or statistical analysis. In some embodiments, the sequencing data provides information regarding two or more molecular interactions.

In other embodiments, information transfer between the associated polypeptide tag and moiety tag to the separate record polynucleotide can be mediated by a ligase, e.g., a DNA ligase, a ssDNA ligase (e.g., Cireligase), a dsDNA ligase, or an RNA ligase. In other embodiments, information transfer between the associated polypeptide tag and the moiety tag to the separate record polynucleotide can be mediated by a topoisomerase. In other embodiments, information transfer between the associated polypeptide tag and moiety tag can be mediated by chemical ligation. In some embodiments, information transfer between the associated polypeptide tag and/or moiety tag to the separate record polynucleotide(s) can be mediated by extension and/or ligation.

In some embodiments, the method forms multiple separate record polypeptides between the polypeptide tag and more than one site of said moiety or between the polypeptide tag and more than one moiety.

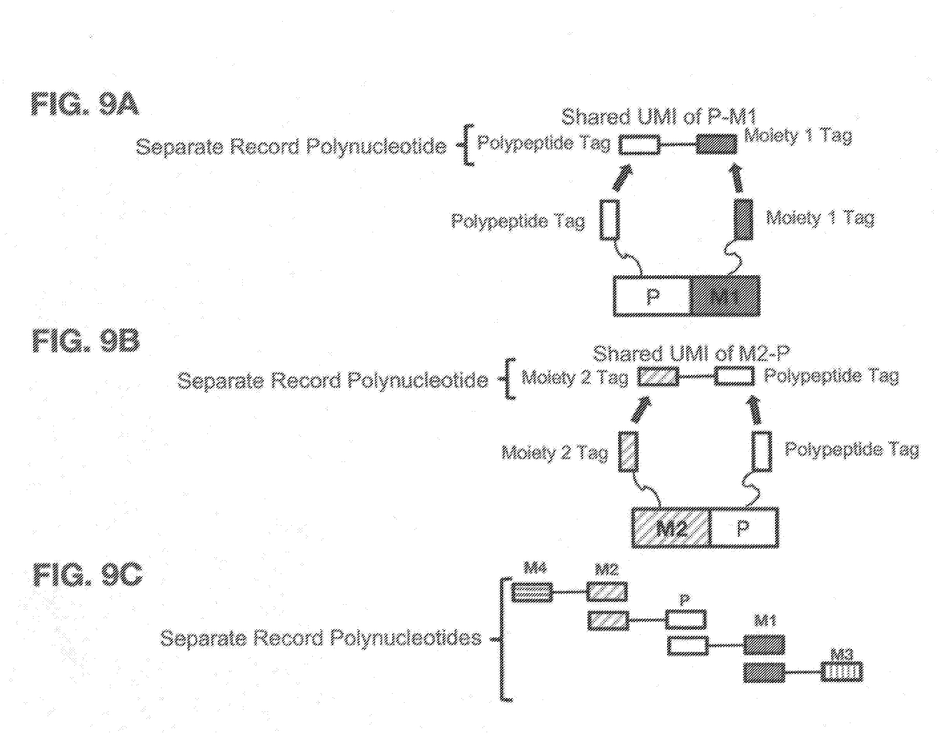

In some embodiments, the linking structure is formed between the site of a polypeptide and one or more sites of a moiety or between the polypeptide tag and one or more moieties. In some embodiments, one or more linking structure(s) is formed between the site of a polypeptide and two or more sites of a moiety or two or more moieties. In some embodiments, the linking structure(s) is formed between the site of a polypeptide and 1, 2, 3, 4, 5, 6, 7, 8, 9, 10 or more sites of a moiety or between the site of a polypeptide and 1, 2, 3 4, 5, 6, 7, 8, 9, 10 or more moieties. In some embodiments, the sites of the moieties each belong to a different polypeptide or protein. In some embodiments, the sites of the moieties are each a different site on a polypeptide. In some examples, the linking structure is formed between the site of a polypeptide and the site of moiety 1, between the site of the polypeptide and the site of moiety 2, between the site of the polypeptide and the site of moiety 3, etc. In some embodiments, the same site of a polypeptide can form, in a pairwise manner, a linking structure with more than one site on the moiety or with more than one moiety (see e.g., FIG. 9A-9C). In some embodiments, a first linking structure is formed between the polypeptide and a first moiety (M1), dissociated, and a second or subsequent linking structure is formed between the polypeptide and a second or subsequent moiety (M2). In some embodiments, the overlapping UMI and/or barcode indicates that the polypeptide formed a linking structure with M1 and M2. In some embodiments, the information from the two or more shared UMI and/or barcodes indicates that the site of the polypeptide and the site of each of the moieties, M1 and M2, are in spatial proximity. In some examples, indirect or overlapping pairwise information from two or more separate record polynucleotides indicates spatial proximity information for the polypeptide with two or more moieties (FIG. 9C).

Transferring information between tie associated polypeptide tag and the moiety tag or ligating the associated polypeptide tag and the moiety tag can form any suitable number of the shared unique molecule identifier (UMI) and/or barcode. For example, transferring information between the associated polypeptide tag and the moiety tag or ligating the associated polypeptide tag and the moiety tag can form a single shared unique molecule identifier (UMI) and/or barcode. The single shared unique molecule identifier (UMI) and/or barcode can comprise any suitable substance or sequence. In some embodiments, the single shared unique molecule identifier (UMI) and/or barcode can be formed by combining multiple sequences, e.g., multiple UMIs and/or barcodes from the polypeptide tag and/or the moiety tag. In some examples, the shared UMI and/or barcode is a composite tag or composite UMI that comprises the sequence of the UMI and/or barcode of the polypeptide tag and the sequence of the UMI and/or barcode of the moiety tag. In another example, transferring information between the associated polypeptide tag and the moiety tag or ligating the associated polypeptide tag and the moiety tag can form a plurality of shared unique molecule identifiers (UMI) and/or barcodes.

The UMI can comprise any suitable substance or sequence. In some embodiments, the UMI has a suitably or sufficiently low probability of occurring multiple times in the sample by chance. In other embodiments, the UMI comprises a polynucleotide comprising from about 3 nucleotides to about 40 nucleotides. The nucleotides in the UMI polynucleotide may or may not be contiguous. In still other embodiments, the polynucleotide in the UMI comprises a degenerate sequence. In yet other embodiments, the polynucleotide in the UMI does not comprise a degenerate sequence. In yet other embodiments, the UMI comprises a nucleic acid, an oligonucleotide, a modified oligonucleotide, a DNA molecule, a DNA with pseudo-complementary bases, a DNA with protected bases, an RNA molecule, a BNA molecule, an XNA molecule, a LNA molecule, a PNA molecule, aγPNA molecule, a morpholino DNA, or a combination thereof. The DNA molecule can be backbone modified, sugar modified, or nucleobase modified. The DNA molecule can also have a nucleobase protecting group such as Alloc, an electrophilic protecting group such as thiarane, an acetyl protecting group, a nitrobenzyl protecting group, a sulfonate protecting group, or a traditional base-labile protecting group including Ultramild reagent.

The polypeptide tag and the moiety tag can be dissociated from each other using any suitable techniques or procedures. For example, if the polypeptide tag and the moiety tag are associated with each other via polypeptide-polypeptide, polypeptide-polynucleotide or polynucleotide-polynucleotide interaction, the polypeptide tag and the moiety tag can be dissociated from each other using any techniques or procedures suitable for breaking such polypeptide-polypeptide, polypeptide-polynucleotide or polynucleotide-polynucleotide interaction. In some embodiments, in the linking structure, the shared UMI and/or barcode comprises a complementary polynucleotide hybrid, and dissociating the polypeptide tag from the moiety tag comprises denaturing the complementary polynucleotide hybrid.

The polypeptide and the moiety can be dissociated from each other using any suitable techniques or procedures. For example, if the polypeptide and the moiety are associated with each other via polypeptide-polypeptide or polypeptide-polynucleotide interaction, the polypeptide and the moiety can be dissociated from each other using any techniques or procedures suitable for breaking such polypeptide-polypeptide or polypeptide-polynucleotide interaction. In some embodiments, both the polypeptide and the moiety are parts of a larger polypeptide, and dissociating the polypeptide from the moiety comprises fragmenting the larger polypeptide into peptide fragments. The larger polypeptide can be fragmented using any suitable techniques or procedures. For example, the larger polypeptide can be fragmented into peptide fragments by a protease digestion. Any suitable protease can be used. For example, the protease can be an exopeptidase such as an aminopeptidase or a carboxypeptidase. In another example, the protease can be an endopeptidase or endoprotehiase such as trypsin, LysC, LysN, ArgC, chymotrypsin, pepsin, thermolysin, papain, or elastase. (See e.g., Switzar, Giera et al. 2013.) In some embodiments, the assessing of at least a partial sequence of the polypeptide and at least a partial identity of the moiety is performed after the polypeptide and moiety are dissociated from each other. For example, the dissociated polypeptide and moiety can be used in a peptide or polypeptide sequencing assay (e.g., a degradation-based polypeptide sequencing assay by construction of an extended recording tag). In some cases, the dissociated polypeptide and moiety can be used in an assay which comprises cyclic removal of a terminal amino acid.

The present methods can be used for assessing identity and spatial relationship between a polypeptide and a moiety in a sample, regardless whether the polypeptide and the moiety belong to the same molecule or not. For example, the target polypeptide and the moiety can belong to two different molecules. In another example, the target polypeptide and the moiety can be parts of the same molecule.

In some embodiments, the target polypeptide is a part of a larger polypeptide and the moiety is also part of the same larger polypeptide. The moiety can be any suitable substance or a complex thereof. For example, the moiety can comprise an amino acid or a polypeptide. The moiety amino acid or polypeptide can comprise one or more modified amino acid(s). Exemplary modified amino acid(s) includes a glycosylated amino acid, a phosphorylated acid, a methylated amino acid, an acylated amino acid, a hydroxyproline or a sulfated amino acid. The glycosylated amino acid can comprise a N-linked or an O-linked glycosyl moiety. The phosphorylated amino acid can be phospholyrosine, phosphoserine or phosphothreonine. The acylated amino acid can comprise a farnesyl, a myristoyl, or a palmitoyl moiety. The sulfated amino acid can be a sulfotyrosine or a part of a disulfide bond.

In other embodiments, the moiety can be a part of a molecule that is bound to, complexed with or in close proximity with the polypeptide in the sample. The moiety can be any suitable substance or a complex thereof. For example, the moiety carr be an atom, an amino acid, a polypeptide, a nucleoside, a nucleotide, a polymicleotide, a vitamin, a monosaccharide, arr oligosaccharide, a carbohydrate, a lipid or a complex thereof. In specific embodiments, the moiety comprises an amino acid or a polypeptide. The moiety amino acid or polypeptide can comprise one or more modified amino acid(s). Exemplary modified amino acid(s) includes a glycosylated amino acid, a phosphorylated amino acid, a methylated amino acid, an acylated amino acid, a hydroxyproline or a sulfated amino acid. The glycosylated amino acid can comprise a N-linked or an O-linked glycosyl moiety. The phosphorylated amino acid can be phosphotyrosine, phosphoserine or phosphothreonine. The acylated amino acid can comprise a fartiesyl, a myristoyl, or a palmitoyl moiety. The sulfated amino acid can be a sulfotyrosine or a part of a disulfide bond.

In some embodiments, the polypeptide and the moiety can belong to two different proteins in the same protein complex. In other embodiments, the moiety can be a part of a poly nucleotide molecule, e.g., a DNA or a RNA molecule, that is bound to, complexed with or in close proximity with the polypeptide in the sample.

The polypeptide tag, the moiety tag, at least a partial sequence of the polypeptide, and/or at least a partial identity of the moiety can be assessed using any suitable techniques or procedures. For example, if the polypeptide tag, the moiety and/or the moiety tag comprises a polypeptide and/or a polynucleotide, any suitable techniques or procedures for assessing identity or sequence of a polypeptide and/or a polynucleotide can be used. Similarly, any suitable techniques or procedures for assessing a polypeptide can be used to assess at least a partial sequence of the polypeptide.

In some embodiments, the polypeptide tag and/or the moiety tag comprises a polypeptide(s), the polypeptide tag and/or the moiety tag can be assessed using a binding assay, e.g., an immunoassay. Exemplary immunoassays include an enzyme-linked immunosorbent assay (ELISA), immunoblotting, immunoprecipitation, radioirrimunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

In some embodiments, the polypeptide tag and/or the moiety tag comprises a polynucleotide, e.g., DNA or RNA. Before or concurrently with the assessment, the polynucleotide can be amplified. The polynucleotide in the polypeptide tag and/or the moiety tag can be amplified using any suitable techniques or procedures. For example, polynucleotide can be amplified using a procedure of polymerase chain reaction (PCR), strand displacement amplification (SDA), transcription mediated amplification (TMA), ligase chain reaction (LCR), nucleic acid sequence based amplification (NASBA), primer extension, rolling circle amplification (RCA), self-sustained sequence replication (3SR), or loop-mediated isothermal amplification (LAMP).

At least a partial sequence of the polypeptide or at least a partial identity of the moiety can be assessed using any suitable techniques or procedures. If the moiety comprises polypeptide, at least a partial sequence of the both of the polypeptide and the moiety can be assessed by any suitable polypeptide sequencing techniques or procedures. For example, at least a partial sequence of the both of the polypeptide and the moiety can be assessed by N-terminal amino acid analysis, C-terminal amino acid analysis, the Edman degradation, and identification by mass spectrometry. In some embodiments, at least a partial sequence of one or both of the polypeptide and the moiety can be assessed by using cognate binding agents (e.g., antibodies or mixed population of monoclonal antibodies) that bind or recognize at least a portion of a macromolecule. In another example, at least a partial sequence of both of the polypeptide and the moiety can be assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Application No. PCT/US2017/030702, published as WO 2017/192633 A1. In some embodiments, the polypeptide and moiety are dissociated from each other and immobilized on a support prior to assessing at least a partial sequence of the polypeptide and/or at least partial identity of the moiety. In some aspects, the assessing of at least a partial sequence of the polypeptide or at least a partial identity of the moiety is performed using a method that includes or uses DNA and/or DNA encoding.

In some embodiments, the at least a partial sequence of the polypeptide is assessed using a procedure comprising: a1) providing the polypeptide and the associated polypeptide tag that serves as a recording tag; b1) contacting the polypeptide with a first binding agent capable of binding to the polypeptide, wherein the first binding agent comprises a first coding tag with identifying information regarding the first binding agent; c1) transferring the information of the first coding tag to the recording tag to generate a first order extended recording tag; and d1) analyzing the first order extended recording tag. The step a1) can comprise providing the polypeptide and an associated polypeptide tag joined to a solid support. The method can further comprise contacting the polypeptide with a second (or higher order) binding agent comprising a second (or higher order) binding portion capable of binding to the polypeptide and a coding tag with identifying information regarding the second (or higher order) binding agent, transferring the information of the second (or higher order) coding tag to the first order extended recording tag to generate a second order (or higher order) extended recording tag, and analyzing the second order (or higher order) extended recording tag.

In some embodiments, the at least a partial sequence of the polypeptide is assessed using a procedure comprising: a1) providing the polypeptide and the associated polypeptide tag that serves as a recording tag; b1) contacting the polypeptide with a first binding agent capable of binding to the N-terminal amino acid (NTAA) of the polypeptide, wherein the first binding agent comprises a first coding tag with identifying information regarding the first binding agent; c1) transferring the information of the first coding tag to the recording tag to generate an extended recording tag; and d1) analyzing the extended recording tag. The method can farther comprise providing the polypeptide and an associated polypeptide tag joined to a solid support. The method can farther comprise contacting the target polypeptide with a second (or higher order) binding agent comprising a second (or higher order) coding tag with identifying information regarding the second (or higher order) binding agent, wherein the second (or higher order) binding agent is capable of binding to a NTAA other than the NTAA of the polypeptide. The contact between the polypeptide with the second (or higher order) binding agent can be conducted in any suitable manner. For examle, contacting the polypeptide with the second (or higher order) binding agent can occur in sequential order following the polypeptide being contacted with the first binding agent. In another example, contacting the polypeptide with the second (or higher order) binding agent can occur simultaneously with the polypeptide being contacted with the first binding agent.

In some embodiments, the at least a partial sequence of the polypeptide is assessed using a procedure comprising: a1) providing, the polypeptide and the associated polypeptide tag that serves as a recording tag; b1) contacting the polypeptide with a first binding agent capable of binding to the N-terminal amino acid (NTAA) of the polypeptide, wherein the first binding agent comprises a first coding tag with identifying information regarding the first binding agent; c1) transferring the information of the first coding tag to the recording tag to generate a first order extended recording tag; d1) removing the NTAA to expose a new NTAA of the target polypeptide; e1) contacting the polypeptide with a second (or higher order) binding agent comprising a second (or higher order) coding tag with identifying information regarding the second (or higher order) binding agent, wherein the second (or higher order) binding agent is capable of binding to the new NTAA, wherein the second (or higher order) binding agent comprises a second coding tag with identifying information regarding the second (or higher order) binding agent; f1) transferring the information of the second (or higher order) coding tag to the first extended recording tag to generate a second order (or higher order) extended recording lag; and g1) analyzing the second order (or higher order) extended recording tag. The steps d1)-g1) can be repeated one or more times. The method can further comprise providing the polypeptide and the associated polypeptide tag joined to a solid support.

In some embodiments, the at least a partial sequence of the polypeptide is assessed using a procedure comprising: a1) providing toe polypeptide and the associated polypeptide tag that serves as a recording tag; b1) modifying the N-terminal amino acid (NTAA) of the polypeptide, e.g., with a chemical agent; c1) contacting the polypeptide with a first binding agent capable of binding to the modified NTAA, wherein the first binding agent comprises a first coding tag with identifying information regarding the first binding agent; d1) transferring the information of the first coding tag to the recording tag to generate a first order extended recording tag; and e1) analyzing the first order extended recording tag. The step a1) can comprise providing the polypeptide and an associated polypeptide tag joined to a solid support. The method can further comprise contacting the polypeptide with a second (or higher order) binding agent comprising a second (or higher order) coding tag with identifying information regarding die second (or higher order) binding agent, wherein the second (or higher order) binding ageut is capable of binding to a modified NTAA other than die modified NTAA of step b1). The contact between the polypeptide and the second (or higher order) binding agent can be conducted in any suitable manner. For example, contacting the polypeptide with the second (or higher order) binding agent can occur in sequential order following the polypeptide being contacted with the first binding agent. In another example, contacting the polypeptide with the second (or higher order) binding agent can occur simultaneously with the polypeptide being contacted with the first binding agent.

In some embodiments, analyzing the first order and/or the second (or higher order) extended recording tag also assesses the polypeptide tag.

In some embodiments, the moiety comprises a moiety polypeptide, and at least a partial identity or sequence of the moiety can te assessed using a procedure comprising: a2) providing the moiety polypeptide and the associated moiety tag that serves as a recording tag; b2) contacting the moiety polypeptide with a first binding agent capable of binding to the moiety polypeptide, wherein the first binding agent comprises a first coding tag with identifying information regarding the first binding agent; c2) transferring the information of die first coding tag to the recording tag to generate a first order extended recording tag; and d2) analyzing the first order extended recording tag. The method can further comprise contacting the moiety polypeptide with a second (or higher order) binding agent comprising a second (or higher order) binding portion capable of binding to the moiety polypeptide and a coding tag with identifying information regarding the second (or higher order) binding agent, transferring the information of the second (or higher order) coding tag to the first order extended recording tag to generate a second order (or higher order) extended recording tag, and analyzing the second order (or higher order) extended recording tag.

In some embodiments, the at least a partial sequence of the moiety polypeptide is assessed using a procedure comprising: a2) providing the moiety polypeptide and the associated moiety tag that serves as a recording tag; b2) contacting the moiety polypeptide with a first binding agent capable of binding to the N-terminal amino acid (NTAA) of the moiety polypeptide, wherein the first binding agent comprises a first coding tag with identifying information regarding the first binding agent; c2) transferring the information of the first coding tag to the recording tag to generate an extended recording tag; and d2) analyzing the extended recording tag. The method can further comprise providing the moiety polypeptide and an associated moiety tag joined to a solid support. The method can further comprise contacting the moiety poiypeptide with a second (or higher order) binding agent comprising a second (or higher order) coding tag with identifying information regarding the second (or higher order) binding agent, wherein the second (or higher order) binding agent is capable of binding to a NTAA other than the NTAA of the polypeptide. The contact between the moiety polypeptide with the second (or higher order) binding agent can be conducted in any suitable manner. For example, contacting the moiety polypeptide with the second (or higher order) binding agent can occur in sequential order following the moiety polypeptide being contacted with the first binding agent. In another example, contacting the moiety polypeptide with the second (or higher order) binding agent can occur simultaneously with the moiety polypeptide being contacted with the first binding agent.

In some embodiments, the at least a partial sequence of the moiety polypeptide is, assessed using a procedure comprising: a2) providing the moiety polypeptide and the associated moiety tag that serves as a recording tag; b2) contacting the moiety polypeptide with a first binding agent capable of binding to the N-terminal amino acid (NTAA) of the moiety polypeptide, wherein the first binding agent comprises a first coding tag with identifying information regarding the first binding agent; c2) transferring the information of the first coding tag to the recording tag to generate a first order extended recording tag; d2) removing the NTAA to expose a new NTAA of the moiety polypeptide; e2) contacting the moiety polypeptide with a second (or higher order) binding agent comprising a second (or higher order) coding tag with identifying information regarding the second (or higher order) binding agent, wherein the second (or higher order) binding agent is capable of binding to the new NTAA, wherein the second (or higher order) binding agent comprises a second coding tag with identifying information regarding the second (or higher order) binding agent; f2) transferring the information of the second (or higher order) coding tag to the first extended recording tag to generate a second order (or higher order) extended recording tag; and g2) analyzing the second order (or higher order) extended recording tag. The steps d2)-g2) can be repeated one or more times. The method can further comprise providing the moiety polypeptide and the associated moiety tag joined to a solid support.

In some embodiments, the at least a partial sequence of the moiety polypeptide is assessed using a procedure comprising: a2) providing the moiety polypeptide and the associated moiety tag that serves as a recording tag; b2) modifying the N-terminal amino acid (NTAA) of the moiety polypeptide, e.g., with a chemical agent; c2) contacting the moiety polypeptide with a first binding agent capable of binding to the modified NTAA, wherein the first binding agent comprises a first coding tag with identifying information regarding the first binding agent; d2) fransferring the information of the first coding tag to the recording tag to generate a first order extended recording tag; and e2) analyzing the first order extended recording tag. The step a2) can comprise providing the moiety polypeptide and the associated moiety tag joined to a solid support. The method can further comprise contacting the moiety polypeptide with a second (or higher order) binding agent comprising a second (or higher order) coding tag with identifying information regarding the second (or higher order) binding agent, wherein the second (or higher order) binding agent is capable of binding to a modified NTAA other than the modified NTAA of step b2). The contact between the moiety polypeptide and the second (or higher order) binding agent can be conducted in any suitable manner. For example, contacting the moiety polypeptide with the second (or higher order) binding agent can occur in sequential order following the moiety polypeptide being contacted with the first binding agent. In another example, contacting the moiety polypeptide with the second (or higher order) binding agent can occur simultaneously with the moiety polypeptide being contacted with the first binding agent.

In some embodiments, the methods described herein use a binding agent capable of binding to the macromolecule, e.g., the polypeptide or the moiety. A binding agent can be any molecule (e.g., peptide, polypeptide, protein, nucleic acid, carbohydrate, small molecule, and the like) capable of binding to a component or feature of a polypeptide. A binding agent can be a naturally occurring, synthetically produced, or recombinantly expressed molecule. In some embodiments, the scaffold used to engineer a binding agent can be from any species, e.g., human, non-human, transgenic. A binding agent may hind to a single monomer or subunit of a polypeptide (e.g., a single amino acid) or bind to multiple linked subunits of a polypeptide (e.g., dipeptide, tripeptide, or higher order peptide of a longer polypeptide molecule) or bind to an epitope.

In certain embodiments, a binding agent may be designed to bind covalently. Covalent binding can be designed to be conditional or favored upon binding to the correct moiety. For example, an NTAA and its cognate NTAA-specific binding agent may each be modified with a reactive group such that once the NTAA-specific binding agent is bound to the cognate NTAA, a coupling reaction is carried out to create a covalent linkage between the two. Non-specific binding of the binding agent to other locations that lack the cognate reactive group would not result in covalent attachment. In some embodiments, the polypeptide comprises a ligand that is capable of forming a covalent bond to a binding agent. In some embodiments, the polypeptide comprises a functionalized NTAA which includes a ligand group that is capable of covalent binding to a binding agent. Covalent binding between a binding agent and its target may allow for more stringent washing to be used to remove binding agents that are non-specifically bound.

In certain embodiments, a binding agent may be a selective binding agent. As used herein, selective binding refers to the ability of the binding agent to preferentially bind to a specific ligand (e.g., amino acid or class of amino acids) relative to binding to a different ligand (e.g., amino acid or class of amino acids). Selectivity is commonly referred to as the equilibrium constant for the reaction of displacement of one ligand by another ligand in a complex with a binding agent. Typically, such selectivity is associated with the spatial geometry of the ligand and/or the manner and degree by which the ligand binds to a binding agent, such as by hydrogen bonding or Van der Waals forces (non-covalent interactions) or by reversible or non-reversible covalent attachment to the binding agent. It should also be understood that selectivity may be relative, and as opposed to absolute, and that different factors can affect the same, including ligand concentration. Thus, in one example, a binding agent selectively binds one of the twenty standard amino acids. In some examples, a binding agent binds to an N-temlinal amino acid residue, a C-terminal amine acid residue, or an internal amino acid residue.

In some embodiments, the binding agent is partially specific or selective. In some aspects, the binding agent preferentially binds one or more amino acids. In some examples, a binding agent may bind to two or more of the twenty standard amino acids. For example, a binding agent may preferentially bind the amino acids A, C, and G over other amino acids. In some other examples, the binding agent may selectively or specifically bind more than one amino acid. In some aspects, the binding agent may also have a preference for one or more amino acids at the second, third, fourth, fifth, etc. positions from the terminal amino acid. In some cases, the binding agent preferentially binds to a. specific terminal amino acid and one or more penultimate amino acid. In some cases, the binding agent preferentially binds to one or more specific terminal amino acid(s) and one penultimate amino acid. For example, a binding agent may preferentially bind AA, AC, and AG or a binding agent may preferentially bind AA, CA, and GA. In some specific examples, binding agents with different specificities can share the same coding tag. In some embodiments, a binding agent may exhibit flexibility and variability in target binding preference in some or all of the positions of the targets. In some examples, a binding agent may have a preference for one. or more specific target terminal amino acids and have a flexible preference for a target at the penultimate position. In some other examples, a binding agent may have a preference for ono or more specific target amino acids in the penultimate amino acid position and have a flexible preference for a target at the terminal amino acid position. In some embodiments, a binding agent is selective for a target comprising a terminal amino acid and other components of a macromolecule, in some examples, a binding agent is selective for a target comprising a terminal amino acid and at least a portion of the peptide backbone. In some particular examples, a binding agent is selective for a target comprising a terminal amino acid and an amide peptide backbone. In some cases, the peptide backbone comprises a natural peptide backbone or a post-translational modification. In some embodiments, the binding agent exhibits allosteric binding.

In the practice of the methods disclosed herein, the ability of a binding agent to selectively bind a feature or component of a macromolecule, e.g., a polypeptide, need only be sufficient to allow transfer of its coding tag information to the recording tag associated with the polypeptide. Thus, selectively need only be relative to the other binding agents to which the polypeptide is exposed. It should also be understood that selectivity of a binding agent need not be absolute to a specific amino acid, but could be selective to a class of amino acids, such as ammo acids with polar or non-polar side chains, or with electrically (positively or negatively) charged side chains, or with aromatic side chains, or some specific class or size of side chains, and the like. In some embodiments, the ability of a binding agent to selectively bind a feature or component of a macromolecule is characterized by comparing binding abilities of binding agents. For example, the binding ability of a bindi ng agent to the target can be compared to the binding ability of a binding agent which binds to a different target, for example, comparing a binding agent selective for a class of amino acids to a binding agent selective for a different class of amino acids. In some examples, a binding agent selective for non-polar side chains is compared to a binding agent selective for polar side chains. In some embodiments, a binding agent selective for a feature, component of a peptide, or one or More amino acid exhibits at least 1×, at least 2×, at least 5×, at least 10×, at least 50×, at least 100×, or at least 500× more binding compared to a binding agent selective for a different feature, component of a peptide, or one or more amino acid.

In a particular embodiment, the binding agent has a high affinity and high selectivity for the macromolecule. In particular, a high binding affinity with a low off-rate may be efficacious for information transfer between the coding tag and recording tag. In certain embodiments, a binding agent has a Kd of about <500 nM, <200 nM, <100 nM, <50 nM, <10 nM, <5 nM, <1 nM, <0.5 nM, or <0.1 nM. In some cases, a binding agent has a Kd of about <100 nM. In a particular embodiment, the binding agent is added to the polypeptide at a concentration >10×, >100×, or >1000× its Kd to drive binding to completion. For example, binding kinetics of an antibody to a single protein molecule is described in Chang et al., J Immunol Methods (2012) 378(1-2): 102-115.

In certain embodiments, a binding agent may bind to an NTAA, a CTAA, an intervening amino acid, dipeptide (sequence of two amino acids), tripeptide (sequence of three amino acids), or higher order peptide of a peptide molecule. In some embodiments, each binding agent in a libruy of binding agents selectively binds to a particular amino acid, for example one of the twenty standard naturally occuning amino acids. The standard, naturally occurring amino acids include Alanine (A or Ala), Cysteine (C or Cys), Aspartic Acid (D or Asp), Glutamic Acid (E or Glu), Phenylalaninw (F or Phe), Glycine (G or Gly):Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). In some embodiments, the binding agent binds to an unmodified or native amino acid. In some examples, the binding agent binds to an unmodified or native dipeptide (sequence of two amino acids), tripeptide (sequence of three amino acids), or higher order peptide of a peptide molecule. A binding agent may be engineered for high affinity for a native or unmodified NTAA, high specificity for a native or unmodified NTAA, or both. In some embodiments, binding agents can be developed through directed evolution of promising affinity scaffolds using phage display.

In some embodiments, a binding agent may bind to a native or unmodified of unlabeled terminal amino acid. In certain embodiments, a binding agent may bind to a modified or labeled terminal amino acid (e.g., an NTAA that has been functionalized or modified). In some embodiments, a binding agent may bind to a chemically or enzymatically modified terminal amino acid. A modified or labeled NTAA can be one that is functionalized with PITC, 1-fluoro-2,4-dinitrobenzene (Sanger's reagent, DNFB), benzyloxycarbonyl chloride or carbobenzoxy chloride (Cbz -Cl), N-(Benzyjoxycarborlyloxy)succinimide (Cbz-OSu or Cbz-O-NHS), dansyl chloride (DNS-Cl, or 1-dimethylaminonaphthalene-5-sulfonyl chloride), 4-sulfonyl-2-nitrofluorobenzene (SNFB), an acetylating reagent, a guanidinylation reagent, a thioacylation reagent, a thioacetylation reagent, or a thiobenzylation reagent. In some examples, the binding agent binds an amino acid labeled by contacting with a reagent or using a method as described in international Patent Publication No. WO 2019/089846. In some cases, the binding agent binds an amino acid labeled by an amine modifying reagent.

In some embodiments, the, binding agent is derived from a biological, naturally occurring, non-naturally occurring, or synthetic source. In some examples, the binding agent is derived from de novo protein design (Huang et al., (2016) 537(7620):320-327). In some examples, the binding agent has a siructure, sequence, and/or activity designed from first principles. In certain embodiments, a binding agent can be an aptamer (e.g., peptide aptamer, DNA aptamer, or RNA aptamer), a peptoid, an amino acid binding protein or enzyme, an antibody or a specific binding fragment thereof, an antibody binding fragment, an antibody mimetic, a peptide, a pcptidomimctic, a protein, or a polynucleotide (e.g., DNA, RNA, peptide nucleic acid (PNA), a gPNA, bridged nucleic acid (BNA), xeno nucleic acid (XNA), glycerol nucleic acid (GNA), or thrcosc nucleic acid (TNA), or a variant thereof).

Potential scaffolds that can be engineered to generate binding agents for use in the methods described herein include: an anticalin, a lipocalin, an amino acid tRNA synthetase (aaRS), CIpS, an Affilin®), an Adnectin™, a T cell receptor, a zinc finger protein, a thiorcdoxin, GST A1-1, DARPin, an affimer, an affitin, an alphabody, an avimer, a Kunitz domain peptide, a monobody, an antibody, a single domain antibody, a nanobody, EETl-II, HPSTI, intrabody. PHD-finger, V(NAR) LDTI, evibody, Ig(NAR), knottin, maxibody, microbody, neocarzinostatin. pVIII, tendamistat, VLR, protein A scaffold, MTI-II, ccotin. GCN4, Im9, kunitz domain, PBP, trans-body, tetranectin, WW domain, CBM4-2, DX-88, GFP, tMab, Ld1 receptor domain A, Min-23, PDZ-domain, avian pancreatic polypeptide, charybdotoxin/10Fn3, domain antibody (Dab), a2p8 ankyrin repeat, insect defending A peptide. Designed AR protein, C-type lectin domain, staphylococcal nuclease, Src homology domain 3 (SH3), or Src homology dotnain 2 (SH2). In some embodiments, a binding agent is derived from an enzyme which binds one or more amino acids (e.g., an aminopeptiduse). In certain embodiments, a binding agent can be derived from an anticaiin or an ATP-dependent Clp protcasc adaptor protein (ClpS).

In some embodiments, a binding ajcnt comprises a coding tag containing identifying information regarding the binding agent. A coding tag is a nucleic acid molecule of about 3 bases to about 100 bases that provides unique identifying information for its associated binding agent. A coding tag may comprise about 3 to about 90 bases, about 3 to about 80 bases, about 3 to about 70 bases, about 3 to about 60 bases, about 3 bases to about 50 bases, about 3 bases to about 40 bases, about 3 bases to about 30 base, about 3 bases to about 20 bases, about 3 bases to about 10 bases, or about 3 bases to about 8 bases. In some embodiments, a coding tag is about 3 bases, 4 bases, 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 25 bases, 30 bases, 35 bases, 40 bases, 55 bases, 60 bases, 65 bases, 70 bases, 75 bases, 80 bases, 85 bases, 90 bases, 95 bases, or 100 bases in length. A coding tag may be eompascd of DNA, RNA, polynucleotide analogs, or a combination thereof. Polynucleotide analogs include PNA, gPNA, BNA, GNA, TNA, LNA, morpholino polynucleotides, 2′-O-Methyl polynucleotides, alkyl ribosyl substituted polynucleotides, phosphorothioate polynucleotides, and 7-deaza purine analogs.

A coding tag comprises an encoder sequence that provides identifying information regarding the associated binding agent. An encoder sequence is about 3 bases to about 30 bases, about 3 bases to about 20 bases, about 3 bases to about 10 bases, or about 3 bases to about 8 bases. In some embodiments, an encoder sequence is about 3 bases, 4 bases, 5 bases, 6 bases, 7 bases, 8 bases, 9 bases, 10 bases, 11 bases, 12 bases, 13 bases, 14 bases, 15 bases, 20 bases, 25 bases, or 30 bases in length. In some embodiments, the length of the encoder sequence determines the number of unique encoder sequences that can be generated. Shorter encoding sequences generate a smaller number of unique encoding sequences, which may be useful when using a small number of binding agents. In a specific embodiment, a set of >50 unique encoder sequences are used for a binding agent library.

In some embodiments, each unique binding agent within a library of binding agents has a unique encoder sequence. For example, 20 unique encoder sequences may be used for a library of 20 binding agents that bind to the 20 standard amino acids. Additional coding tag sequences may be used to identify modified amino acids (e.g., post-transiationally modified amino acids). In another example, 30 unique encoder sequences may be used for a library of 30 binding agents that bind to the 20 standard amino acids and 10 post-translational modified amino acids (e.g., phosphorylated amino acids, acetylated amino acids, methylated amino acids). In other embodiments, two or more different binding agents may share the same encoder sequence. For example, two binding agents that each bind to a different standard amino acid may share the same encoder sequence.

In certain embodiments, a coding tag further comprises a spacer sequence at one end or both ends. A spacer sequence is about 1 base to about 20 bases, about 1 base to about 10 bases, about 5 bases to about 9 bases, or about 4 bases to about 8 bases. In some embodiments, a spacer is about 1 base, 2 bases, 3 bases, 4 bases, 5 bases, 6 bases, 7 bases, 8 bases, 9 bases, 10 bases, il bases, 12 bases, 13 bases, 14 bases, 15 bases or 20 bases in length. In some embodiments, a spacer within a coding tag is shorter than the encoder sequence, e.g., at least 1 base, 2, bases, 3 bases, 4 bases, 5 bases, 6, bases, 7 bases, 8 bases, 9 bases, 10 bases, 11 bases, 12 bases, 13 bases, 14 bases, 15 bases, 20 bases, or 25 bases shorter than the encoder sequence. In other embodiments, a spacer within a coding tag is the same length as the encoder sequence. In certain embodiments, the spacer is binding agent specific so that a spacer from a previous binding cycle only interacts with a spacer from the appropriate binding agent in a current binding cycle. An example would be pairs of cognate antibodies containing spacer sequences that only allow information transfer if both antibodies sequentially bind to the polypeptide. A spacer sequence may be used as the primer annealing site for a primer extension reaction, or a splint or sticky end in a ligation reaction. A 5′ spacer on a coding tag may optionally contain pseudo complementary bases to a 3′ spacer on the recording tag to increase T, (Lehoud et al., 2008, Nucleic Acids Res. 36:3409-3419). In other embodiments, the coding tags within a library of binding agents do not have a binding cycle specific spacer sequence.

In some embodiments, the coding tags within a collection of binding agents share a common spacer sequence used in an assay (e.g. the entire library of binding agents used in a multiple binding cycle method possess a common spacer in their coding tags). In another embodiment, the coding tags are comprised of a binding cycle tags, identifying a particular binding cycle. In other embodiments, the coding tags within a library of binding agents have a binding cycle specific spacer sequence. In some embodiments, a coding tag comprises one binding cycle specific spacer sequence. For example, a coding tag for binding agents used in the first binding cycle comprise a “cycle 1” specific spacer sequence, a coding tag for binding agents used in the second binding cycle comprise a “cycle 2” specific spacer sequence, and so on up to “n” binding cycles. In further embodiments, coding tags for binding agents used in the first binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. In some embodiments, a spacer sequence comprises a sufficient number of bases to anneal to a complementary spacer sequence in a recording tag or extended recording tag to initiate a grinner extension reaction or sticky end ligation reaction.

In some embodiments, coding tags associated with binding agents used to bind in an alternating cycles comprises different binding cycle specific spacer sequences. For example, a coding tag for binding agents used in the first binding cycle comprise a “cycle 1” specific spacer sequence, a coding tag for binding agents used in the second binding cycle comprise a “cycle 2” specific spacer sequence, a coding tag for binding agents used in the third binding cycle also comprises the “cycle 1” specific spacer sequence, a coding tag for binding agents used in the fourth binding cycle comprises the “cycle 2” specific spacer sequence. In this manner, cycle specific spacers are not needed for every cycle.

A cycle specific spacer sequence can also be used to concatenate information of coding tags onto a single recording tag when a population of recording tags is associated with a polypeptide. The first binding cycle transfers information from the coding tag to a randomly-chosen recording tag, and subsequent binding cycles can prime only the extended recording tag using cycle ependent spacer sequences. More specifically, coding tags for binding agents used in the fast binding cycle comprise a “cycle 1” specific spacer sequence and a “cycle 2” specific spacer sequence, coding tags for binding agents used in the second binding cycle comprise a “cycle 2” specific spacer sequence and a “cycle 3” specific spacer sequence, and so on up to “n” binding cycles. Coding tags of binding agents from the first binding cycle are capable of annealing to recording tags via complementary cycle 1 specific spacer sequences. Upon transfer of the coding tag information to the recording, tag, the cycle 2 specific spacer sequence is positioned at the 3′ terminus of the extended recording tag at the end of binding cycle 1. Coding tags of binding agents from the second binding cycle are capable of annealing, to the extended recording tags via complementary cycle 2 specific spacer sequences. Upon transfer of the coding tag information to the extended recording tag, the cycle 3 specific spacer sequence is positioned at the 3′ terminus of the extended recording tag at the end of binding cycle 2, and so on through “n” binding cycles. This embodiment provides that transfer of binding information in a particular binding cycle among multiple binding cycles will only occur on (extended) recording tags that have experienced the previous binding cycles. However, sometimes a binding agent may fail to bind to a cognate polypeptide. Oligonucleotides comprising binding cycle specific spacers after each binding cycle as a “chase” step can be used to keep the binding cycles synchronized even if the event of a binding cycle failure. For example, if a cognate binding agent fails to bind to a polypeptide during binding cycle 1, adding a chase step followhig binding cycle 1 using oligonucleotides comprising both a cycle 1 specific spacer, a cycle 2 specific spacer, and a “null” encoder sequence. The “null” encoder sequence can be the absence of an encoder sequence or, preferably, a specific barcode that positively identifies a “null” binding cycle. The “null” oligonucleotide is capable of annealing to the recording tag via the cycle 1 specific spacer, and the cycle 2 specific spacer is transferred to the recording tag. Thus, binding agents from binding cycle 2 are capable of annealing to the extended recording tag via the cycle 2 specific spacer despite the failed binding cycle 1 event. The “null” oligonucleotide marks binding cycle 1 as a failed binding event within the extended recording tag.

In some embodiments, a coding tag comprises a cleavable or nickable DNA strand within the second (3′) spacer sequence proximal to the binding agent. For example, the 3′ spacer may have one or more uracil bases that can be nicked by moil-specific excision reagent (USER). USER generates a single nucleotide gap at the location of the uracil. In another example, the 3′ spacer may comprise a recognition sequence for a nicking endonuclease that hydrolyzes only one strand of a duplex. Preferably, the enzyme used for cleaving or nicking the 3′ spacer sequence acts only on one DNA strand (the 3′ spacer of the coding tag), such that the other strand within the duplex belonging to the (extended) recording tag is left intact. These embodiments is particularly useful in assays analysing proteins in their native conformation, as it allows the non-denaturing removal of the binding agent from the (extended) recording tag after primer extension has occurred and leaves a single stranded DNA spacer sequence on the extended recording tag available for subsequent binding cycles.

In certain embodiments, a coding tag may further comprise a unique molecular identifier for the binding agent to which the coding tag is linked.

A coding tag may include a terminator nucleotide incorporated at the 3′ end of the 3′ spacer sequence. After a binding agent binds to a polypeptide and their corresponding coding tag and recording tags anneal via complementary spacer sequences, it is possible for primer extension to transfer information from the coding tag to the recording tag, or to transfer information. from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag may be a single stranded molecule, a double stranded molecule, or a partially double stranded. A coding tag may comprise blunt ends, overhanging ends, or one of each. In some embodiments, a coding tag is partially double stranded, which prevents annealing of the coding tag to internal encoder and spacer sequences in a growing extended recording tag. In some embodiments, the coding tag comprises a hairpin. In certain embodiments, the hairpin comprises mutually complementary nucleic acid regions are connected through a nucleic acid strand. In some embodiments, the nucleic acid hairpin can also further comprise 3′ and/or 5′ single-stranded region(s) extending from the double-stranded stem segment. In some examples, the hairpin comprises a single strand of nucleic acid.

In some embodime,nts, a coding tag n:tay include a terminator nucleotide incorporated at the 3′ end of the 3′ spacer sequence. After a binding agent binds to a macromolecule and their corresponding coding tag and recording tags anneal via complementary spacer sequences, it is possible for primer extension to transfer information from the coding tag to the recording tag, or to transfer information from the recording tag to the coding tag. Addition of a terminator nucleotide on the 3′ end of the coding tag prevents transfer of recording tag information to the coding tag. It is understood that for embodiments described herein involving generation of extended coding tags, it may be preferable to include a terminator nucleotide at the 3′ end of the recording tag to prevent transfer of coding tag information to the recording tag.

A coding tag is joined to a binding agent irestny of indirectly, by any means known in the art, including covalent and non-covalent interactions. In some embodiments, a coding tag may be joined to binding agent enzymatically ar chemically. In some embodiments, a coding tag may be joined to a binding agent via ligation. In other embodiments, a coding tag is joined to a binding agent via affinity binding pairs (e.g., biotin and streptavidin). In some cases, a coding tag may be joined to a binding agent to an unnatural amino ac such as via a covalent interaction with an unnatural amino acid.

In some embodiments, a binding agent is joined to a coding tag via. SpyCatcher-SpyTag interaction. The SpyTag peptide forms an irreversible covalent bond to the SpyCatcher protein via a spontaneous isopeptide linkage, thereby offe.ring a genetically encoded way to create peptide interactions that resist force and harsh conditions (Zakeri et al., 2012, Proc. Natl. Acad. Sci. 109:E690-697; Li et al., 2014, J. Mol. Biol. 426:309-317). A binding agent may be expressed as a fusion protein comprising the SpyCatcher protein. In some embodiments, the SpyCatcher protein is appended on the N-terminus or C-teiminus of the binding agent. The SpyTag peptide can be coupled to the coding tag using standard conjugation chemistries (Bioconjugate Techniques, G. T, Hermanson, Academic Press (2013)). In some embodiments, an enzyme-based strategy is used to join the binding agent to a coding tag. In one example, a protein, e.g., SpyLigase, is used to join the binding agent to the coding tag (Fierer et al., Proc Natl Acad Sci S USA. 2014 Apr. 1; 111(13): E1176-E1181).

In other embodiments, a binding agent is joined to a coding tag via SnoopTag-SnoopCatcher peptide-protein interaction. The SnoopTag peptide forms an isopeptide bond with the SnoopCatcher protein (Veggiani et al., Proc. Natl. Acad. Sci. USA, 2016, 113:1202-1207). A binding agent may be expressed as a fusion protein comprising the SnoopCatcher protein. In some embodiments, the SnoopCatcher protein is appended on the N-terminus or C-terminus of the binding agent. The SnoopTag peptide can be coupled to the coding tag using standard conjugation chemistries.

In yet other embodiments, a binding agent is joined to a coding tag via the HaloTag® protein fusion tag and its chemical ligand. HaloTag is a modified haloalkane dehalogenase designed to covalently bind to synthetic ligands (HaloTag ligands) (Los et al., 2008, ACS Chem. Biol. 3:373-382). The synthetic ligands comprise a chloroalkane linker attached to a variety of useful molecules. A covalent bond forms between the HaloTag and the chloroalkane linker that is highly specific occurs rapidly under physiological conditions, and is essentially irreversible.

In some cases, a binding agent is joined to a coding tag by attaching (conjugating) using an enzyme, such as sortase-mediated labeling (See e.g., Antos at al., Curr Protoc Protein Sci. (2009) CHAPTER 15: Unit-15.3; International Patent Publication No. WO2013003555). The sortase enzyme catalyzes a transpeptidation reaction (See e.g., Falck et al, Antibodies (2018) 7(4):1-19). In some aspects, the binding agent is modified with or attached to one or more N-terminal or C-terminal glycine residues.

In some embodiments, a binding agent is joined to a. coding tag using π-clamp-mediated cysteine bioconjugation (See e.g., Zhang et al., Nat Chem. (2016) 8(2):120-128).

In some embodiments, the binding agent is linked, directly or indirectly, to a multimerization domain. Thus, monomeric, dimeric, and higher order (e.g., 3, 4, 5, or more) multimeric polypeptides comprising one or more binding agents are provided herein. In some specific embodiments, the binding agent is dimeric. In some examples, two polypeptides of the invention can be covalently or non-covalently attached to each other to form a dimer.

In some embodiments, analyzing the first order and/or the second (or higher order) extended recording tag also assesses the moiety tag.

In some embodiments, the first order and/or the second (or higher order) extended recording tag comprises a polynucleotide, e.g., DNA or RNA, and at least a partial sequence of the polynucleotide in the first order and/or the second (or higher order) extended recording tag is assessed to assess the at least a partial sequence of polypeptide and/or the moiety, aud/or to assess the polypeptide tag and/or the moiety tag. The polynucleotide sequence can be assessed using any suitable techniques or procedures. For example, the polynucleotide sequence can be assessed using Maxam-Gilbert sequencing, a chain-termination method, shotgun sequencing, bridge PCR, single-molecule real-time sequencing, ion semiconductor (ion torrent sequencing), sequencing by synthesis, sequencing by ligation (SOLiD sequencing), chain termination (Sanger sequencing), massively parallel signature sequencing (MPSS). polony sequencing, 454 pyrosequeucing, lllumina (Solexa) sequencing, DNA nanoball sequencing, heliscope single molecule sequencing, single molecule real time (SMRT) sequencing, nanoporc DNA sequencing, tunnelling currents DNA sequencing, sequencing by hybridization, sequencing with mass spectrometry, microfluidic Sanger sequencing, a microscopy-based technique. RNAP sequencing, or in vitro vims high-throughput sequencing.

The present methods can be used to assess any suitable type of spatial proximity between a polypeptide and a moiety in a sample. In some embodiments, both the polypeptide and the moiety are parts of a larger polypeptide. In some examples, the larger polypeptide has a primary protein structure, and the polypeptide and the moiety are in spatial proximity in the primary protein structure. In some examples, the larger polypeptide has a secondary, tertiary and/or quaternary protein structures), and the polypeptide and the moiety are in spatial proximity in the secondary, tertiary and/or quaternary protein structure(s).

In other embodiments, the polypeptide and the moiety belong to two different molecules. For example, the polypeptide and the moiety can belong to two different proteins in the same protein complex. In other examples, the moiety can be a part of a polynucleotide molecule, e.g., a DNA or a RNA molecule, that is bound to, complexed with or in close proximity with the polypeptide in the sample. In these embodiments, the present methods can be used to assess any suitable type of spatial proximity between or among different molecules, e.g., spatial proximity between or among different subunits in a protein complex, a protein-DNA complex or a protein-RNA complex.

### II. Methods of Proximity Interaction Analysis Using a Pre-Assembled Structure

In one aspect, the present disclosure provides a method for assessing identity and spatial relationship between a polypeptide and a moiety in a sample, which method comprises: a) providing a pre-assembled structure comprising a shared unique molecule identifier (UMI) and/or barcode in the middle portion flanked by a polypeptide tag on one side and a moiety tag on the other side; b) forming a linking structure between a site of a polypeptide in, a sample and a site of a moiety in said sample by associating said polypeptide tag of said pre-assembled structure to said site of said polypeptide and associating said moiety tag of said pre-assembled structure to said site of said moiety; c) breaking said linking structure via dissociating said polypeptide from said moiety and dissociating said polypeptide tag from said moiety tag, while maintaining association between said polypeptide and sais potypeptide tag, and maintaining association between said moiety and said moiety tag; and d) assessing said polypeptide tag and at least a partial sequence of said polypeptide, and assessing said moiety tag and at least a partial identity of said moiety, wherein said assessed portions of said polypeptide tag and said moiety tag comprise said shared unique molecule identifier (UMI) and/or barcode indicates that said site of said polypeptide and said site of said moiety in said sample are in spatial proximity.

Any suitable moiety can be used in the present methods. For example, the moiety can be an atom, an inorganic moiety, an organic moiety or a complex thereof. The organic moiety can be an amino acid, a polypeptide, e.g., a peptide or a protein, a nucleoside, a nucleotide, a polynucleotide, e.g., an oligonucleotide or a nucleic acid, a vitamin, a monosaccharide, an oligosaccharide, a carbohydrate, a lipid and a complex thereof. In some embodiments, the moiety can comprise a polypeptide. In other embodiments, the moiety can comprise a polynueleotide.

Any suitable polypeptide tag can be used in the present methods. For example, the polypeptide tag can be an atom, an inorganic moiety, an organic moiety or a complex thereof. The organic moiety can be an amino acid, a polypeptide, e.g., a peptide or a protein, a nucleoside, a nucleotide, a polynucleotide, e.g., an oligonucleotide or a nucleic acid, a vitamin, a monosaccharide, an oligosaccharide, a carbohydrate, a lipid and a complex thereof. In some embodiments, the polypeptide tag can comprise a polynucleotide.

Any suitable moiety tag can be used in the present methods. For example, the moiety tag can be an atom, an inorganic moiety, an organic moiety or a complex thereof. The organic moiety can be an amino acid, a polypeptide, e.g., a peptide or a protein, a nucleoside, a nucleotide, a polynucleotide, e.g., an oligonucleotide or a nucleic acid, a vitamin, a monosaccharide, an oligosaccharide, a carbohydrate, a lipid and a complex thereof. In some embodiments, the moiety tag can comprise a polynucleotide.

Both the polypeptide tag and the moiety tag can comprise polynucleotides. In some embodiments, the polypeptide tag comprises a UMI and/or barcode. In some embodiments, the moiety tag comprises a UMI and/or barcode. In some embodiments, the polypeptide tag comprises a first polynucleotide and the moiety tag comprise a second polynucleotide, the first and second polynucleotides comprise a complementary sequence, and the polypeptide tag and the moiety tag are associated via the complementary sequence.

In some embodiments, the pre-assembled structure comprises one or more barcodes or one or more UMIs. In some examples, each pre-assembled structure comprises two barcodes. In some examples, each pre-assembled structure comprises two UMIs. In some embodiments, the relationship or association of the two or more associated UMIs of each pre-assembly is established. In some embodirnents, two or more associated UMIs of the pre-assembled structure is assessed (e.g., sequenced) to establish the relationship or association of the UMIs with each other. In some cases, the two or more UMIs are synthesized as a pre-assembled structure. In some cases, the two or more UMIs are joined (directly if indirectly via a linker) to form a pre-assembled structure, in some embodiments, a pre-assembled snucture is joined to a polypeptide and a moiety in proximity, such as by joining a DNA comprising one UMI of the pre-assembled structure to the polypeptide and a DNA comprising one UMI of the pre-assembled structure to the moiety. In some cases, after joining of the pre-assembled structure to the polypeptide and the moiety, the two or more UMIs of the pre-assembled structure are dissociated from each other (while each UMI main ins association with the polypeptide or the moiety). In some embodiments, the relationship or association of the two or more associated UMIs of each pre-assembled is established before dissociating the UMIs from each other. In some embodiments, the assessing of the two or more associated UMIs is performed before dissociating the UMIs from each other. In some embodiments, the methods includes dissociating the two or more UMIs of a pre-assembled structure and dissociating the polypeptide and the moiety.

In some embodiments, the pre-assembled structure comprises a cleavable or nickable DNA strand (e.g. between a first UMI and a second UMI. For example, the pre-assembled structure may have one or more uracil bases that can be nicked by uracil-specific excision reagent (USER).

In some embodiments, the pre assembled structure comprises complementary sequences of a UMI. In some embodiments, the pre-assembled structure comprises a single stranded DNA, a double stranded DNA complex, a DNA duplex, or a DNA hairpin. In some embodiments, the pre-assembied structure comprising a UMI is synthesized or generated by extension, or ligation from a template sequence in the pre-assembled structure to generate the complementary of the UMI sequence in the preassembied structure.

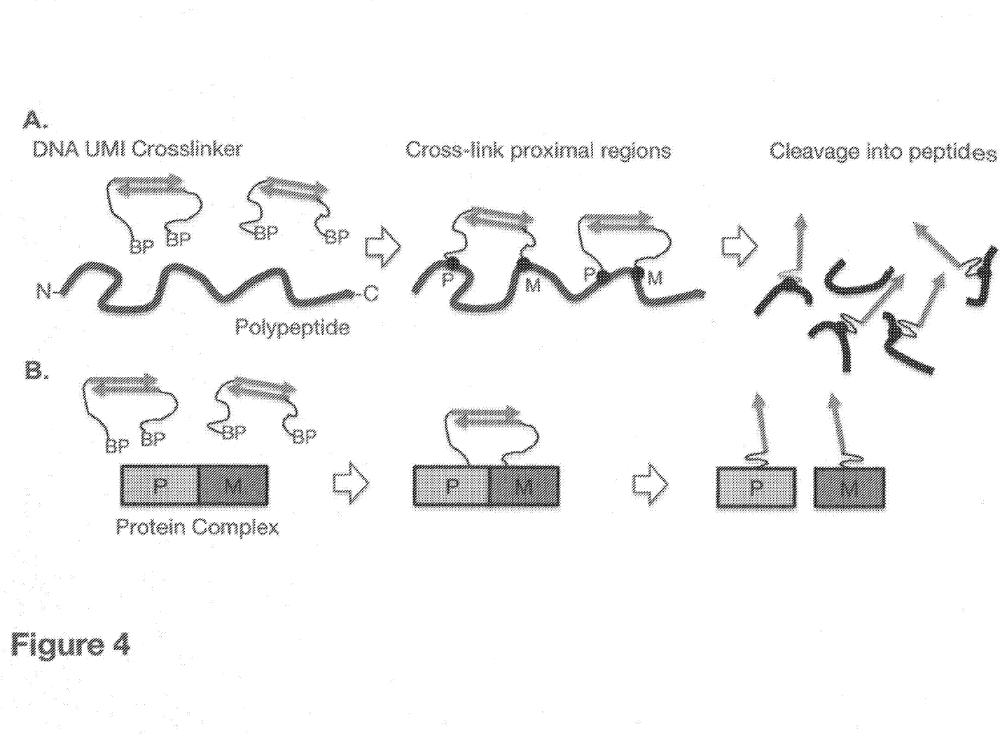

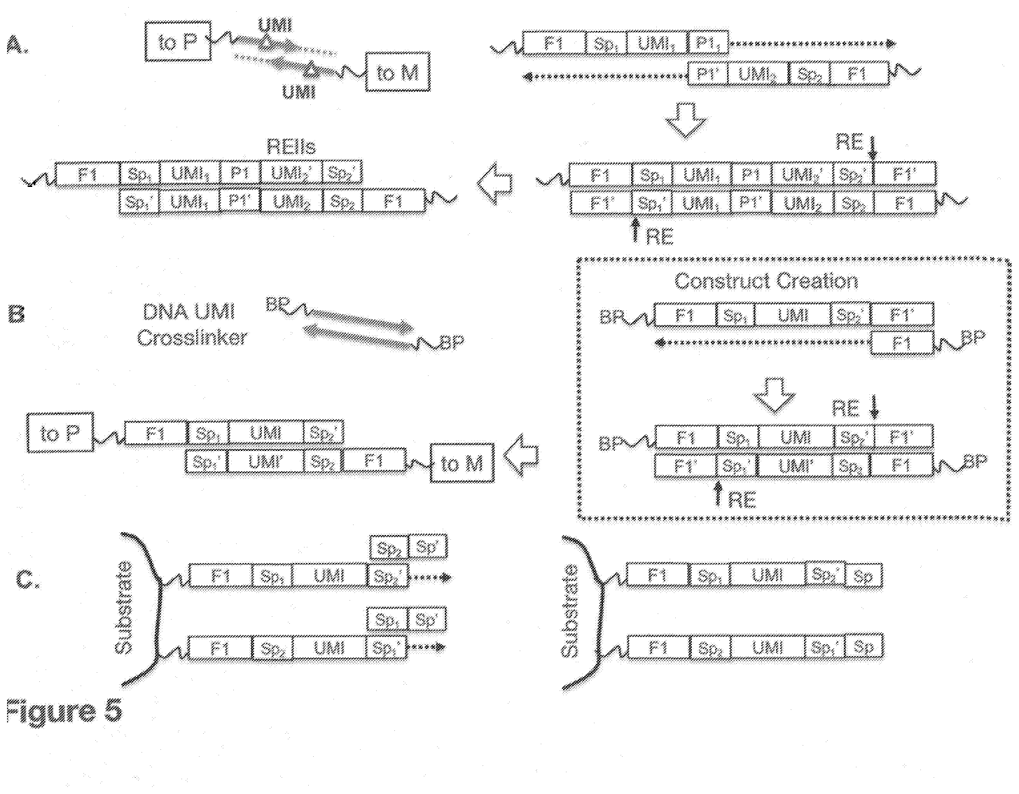

In some embodiments, the methods provide a pre-assembled structure comprising a DNA crosslinker comprising a UMI or a barcode for attaching directly or indirectly to the polypeptide and the moiety in proximity (FIG. 4A-4B). In some examples, a polypeptide and a moiety in proximity labeled with or attached to a DNA complex (e.g., DNA crosslinker) or portion thereof, are dissociated from each other. After dissociation of the polypeptide and the moiety, the polypeptide maintains attachment to one strand of the DNA complex (e.g., DNA crosslinker) comprising the UMI or barcode and the moiety maintains attachment to an at least partially complementary strand of the DNA complex (e.g., DNA crosslinker) containing the UMI or barcode (FIG. 5A-5C). In some embodiments, the DNA complex (e.g., DNA crosslinker (or portion thereof)) is attached directly or indirectly (e.g. to a nucleic acid attached) to the polypeptide and the moiety via enzymatic (e.g. ligation) or chemical methods.

In the linking structure, the polypeptide tag and the moiety tag can be associated in any suitable manner. In some embodiments, in the linking structure, the polypeptide tag and the moiety tag can be associated stably. In other embodiments, in the linking structure, the polypeptide tag and the moiety tag can be associated transiently. The association between the polypeptide tag and the moiety tag can vary over time or over performance of the present methods. In still, other embodiments, in the linking structure, the polypeptide tag and the moiety tag can be associated directly. In yet other embodiments, in the linking structure, the polypeptide tag and the moiety tag can be associated indirectly, e.g., via a linker or UMI between the polypeptide tag and the moiety tag. In some embodiments, the linking structure is formed by associating the polypeptide tag of said pre-assembled structure (e.g., DNA crosslinker) to a site of a polypeptide and associating the moiety tag of said pre-assembled structure to a site of the moiety.

In forming the linking structure, any suitable number of the polypeptide tag(s) can be associated with a suitable number of site(s) of the polypeptide. For example, in forming the linking structure, a single polypeptide tag can be associated witb a single site of the polypeptide, a single polypeptide tag can be associated with a plurality of sites of the polypeptide, or a plurality of the polypeptide tags can be associated with a plurality of sites of the polypeptide. Similarly, in forming the linking structure, any suitable number of die moiety tag(s) can be associated with a suitable number of site(s) of the moiety. For example, in forming the linking structure, a single moiety tag can be associated with a single site of the moictv, a single moiety tag can be associated with a plurality of sites of the moiety, or a plurality of the moiety tags can be associated with a plurality of sites of the moiety.

The formed linking structure can comprise any suitable number of the shared unique molecule identifier (UMI) and/or barcode. For example, the formed linking structure can comprise a single shared unique molecule identifier (UMI) and/or barcode. In another example, the formed linking structure can comprise a plurality of shared unique molecule identifiers (UMI) and/or barcodes. In some examples, the shared UMI and/or barcode is a composite tag or composite UMI that comprises the sequence of the UMI and/or barcode of the polypeptide tag and die sequence of the UMI and/or barcode of the moiety tag.

The UMI and/or the barcode can comprise any suitable substance or sequence. In some embodiments, the UMI has a suitably or sufficiently low probability of occurring multiple times in the sample by chance. In other embodiments, the UMI comprises a polynucleotide comprising from about 3 nucleotides to about −40 nucleotides. The nucleotides in the UMI polynucleotide may or may not lie contiguous. In still other embodiments, the polynucleotide in the UMI comprises a degenerate sequence. In yet other embodiments, the polynucleotide in the UMI docs not comprise a degenerate sequence. In yet other embodiments, the UMI comprises a nucleic acid, an oligonucleotide, a modified oligonucleotide, a DNA molecule, a DNA with pscudo-complcmcntary bases, a DNA with protected bases, an RNA molecule, a DNA molecule, an XNA molecule, a LNA molecule, a PNA molecule, a yPNA molecule, a morpholino DNA, or a combination thereof. The DNA molecule can be backbone modified, sugar modified, or nucleobase modified. The DNA molecule can also have a nucleobase protecting group such as Alloc, an electrophilic protecting group such asthiaranc, an acetyl protecting group, a nitrobenzyl protecting group, a sulfonate protecting group, or a traditional base-labile protecting group including Ultramild reagent.

The polypeptide tag and the moiety tag can be dissociated from each other using any suitable techniques or procedures. For example, if the polypeptide tag and the moiety tag are associated with each other via poiypeptide-polypeptide, polypeptide-polynticleotide or polynucleotide-polynucleotide interaction, the polypeptide tag and the moiety tag can be dissociated from each other using any techniques or procedures suitable for breaking such polypeptide-polypeptide, polypeptide-polynucleotide or polynucleotide-polynucleotide interaction. In some embodiments, in Me linking structure, the shared UNIT and/or barcode comprises a complementary polynucieotide hybrid, and dissociating the polypeptide tag from the moiety tag comprises denaturing the complementary polynueleotide hybrid.

The polypeptide and the moiety can be dissociated from each other using any suitable techniques or procedures. For example, if the polypeptide and the moiety are associated with each other via polypeptide-polypeptide cr polypeptide-polynucleotide interaction, the polypeptide and the moiety can be dissociated from each other using any techniques or procedures suitable for breaking such polypeptide-polypeptide or polypeptide-polynueleotide interaction. In some embodiments, both the polypeptide and the moiety are parts of a larger polypeptide, and dissociating the polypeptide from the moiety comprises fragmenting the larger polypeptide into peptide fimgments. The larger polypeptide can be fragmented using any suitable techniques or procedures. For example, the larger polypeptide can be fragmented into peptide fragments by a protease digestion. Any suitable protease can be used. For example, the protease can be an exopeptidase such as an aminopeptidase or a carboxypeptidase. In another example, the protease can be an endopeptidase cr endoproteinase such as trypsin, LysC, LysN, ArgC, chymotrypsin, pepsin, thermolysin, papain, or elastase. (See e.g., Switzar, Giera et al. 2013.)

The present methods can be used for assessing identity and spatial relationship between a polypeptide and a moiety in a sample, regardless whether the polypeptide and the moiety belong to the same molecule or not. For example, the target polypeptide and the moiety can belong to two different molecules. In another example, the target polypeptide and the moiety can be parts of the same molecule.

In some embodiments, the target polypeptide is a. part of a larger polypeptide and the moiety is also part of the same larger polypeptide. The moiety can be any suitable substance or a complex thereof. For example, the moiety can comprise an amino acid or a polypeptide. The moiety amino acid or polypeptide can comprise one or more modified amino acid(s). Exemplary modified amino acid(s) includes a glycosylated amino acid, a phosphorylated amino acid, a methylated amino acid, an acylated amino acid, a hydroxyproline or a sulfated amino acid. The glycosylated amino acid can comprise a N-linked or an O-linked glycosyl moiety. The phosphorylated amino acid can be phosphotyrosine, phosphoserine or phosphothreonine, The acylated amino acid can comprise a farnesyl, a myristoyl, or a palmitoyl moiety. The sulfated amino acid can be a sulforyrosine, or a part of a disulfide bond.

In other embodiments, the moiety can be a part of a molecule that is bound to, complexed with or in close, proximity with the polypeptide in the sample. The moiety can be any suitable substance or a complex thereof. For example, the moiety can be an atom, an amino acid, a polypeptide, a nucleoside, a nucleotide, a polynucleotide, a vitamin, a monosaccharide, an oligosaccharide, a carbohydrate, a lipid or a complex thereof. In specific embodiments, the moiety comprises an amino acid or a polypeptide. The moiety amino acid or polypeptide can comprise one or more modified amino acid(s). Exemplary modified amino acid(s) includes a glycosylated amino acid, a phosphorylated amino acid, a methylated amino acid, an acylated amino acid, a hydroxyproline or a sulfated amino acid. The glycosylated amino acid can comprise a N-linked or an O-linked glycosyl moiety. The phosphorylated amino acid can be phosphotyrosine, phosphoserine oz phosphothreonine. The acylated amino acid can comprise a farnesyl, a myristoyl, or a palmitoyl moiety. The sulfated amino acid can be a sulfotyrosine or a part of a disulfide bond.

In some embodiments, the polypeptide and the moiety can belong to two different proteins in the same protein complex. In other embodiments, the moiety can be a part of a polynucleotide molecule, e.g., a DNA or a RNA molecule, that is bound to, complexed with or in close proximity with the polypeptide in the sample.

The polypeptide tag, the moiety tag, at least a partial sequence of the polypeptide, and/or at least a partial identity of the moiety can be assessed using any suitable techniques or procedures. For example, if the polypeptide tag, the moiety and/or the moiety tag comprises a polypeptide and/or a polynucleotide, any suitable techniques, or procedures for assessing identity or sequence of a polypeptide and/or a polynucleotide can be used. Similarly, any suitable techniques or procedures for assessing a polypeptide can be used to assess at least a partial sequence of the polypeptide.

In some embodiments, the polypeptide tag and/or the moiety tag comprises a polypeptide(s), the polypeptide tag and/ or moiety tag can be assessed using a binding assay, e.g., an immunoassay. Exemplary immunoassays include an enume-linked immunosorbent assay (ELISA), imrnunoblottmg, immunoprecipitation, radioimmunoassay (RIA), immunostaining, latex agglutination, indirect hemagglutination assay (IHA), complement fixation, indirect immunofluorescent assay (IFA), nephelometry, flow cytometry assay, surface plasmon resonance (SPR), chemiluminescence assay, lateral flow immunoassay, u-capture assay, inhibition assay and avidity assay.

In some embodiments, the polypeptide tag anal/or the moiety tag comprises a polynucleotide, e.g., DNA or RNA. Before or concurrently with the assessment, polynucleotide can be amplified. The polynucleotide in the polypeptide tag and/or the moiety tag can be amplified using any suitable lechaliques or procedures. For example, the polynueleotide can be amplified using a procedure of polymerase chain reaction (PCR), strand displacement amplification (SDA), transcription mediated amplification (TMA), ligase chain reaction (LCR), nucleic acid sequence based amplification (NASBA), primer extension, rolling circle amplification (RCA), self-sustained sequence replication (3SR), or loop-mediated isothermal amplification (LAMP).

At least a partial sequence of the polypeptide or at least a partial identity of the moiety can, be assessed using any suitable techniques or procedures. If the moiety comprises polypeptide, at least a partial sequence of the both of the polypeptide and the moiety can be assessed by any suitable polypeptide sequencing techniques or procedures. For example, at least a partial sequence of the both of the polypeptide and the moiety can be assessed by N-terminal amino acid analysis, C-terminal amino acid analysis, the Edman degradation, and, identification b mass spectrometry. In another example, at least a partial sequence of both of the polypeptide and the moiety can be assessed by the techniques or procedures disclosed and/or claimed in U.S. Provisional Patent Application Nos. 62/330,841, 62/339,071, 62/376,886, 62/579,844, 62/582,312, 62/583,448, 62/579,870, 62/579,840, and 62/582,916, and International Patent Application No. PCT/US2017/030702, published as WO 2017/192633 A1. For example, any techniques or procedures for assessing a macromolecule (e.g. a polypeptide) provided herein, e.g., described in Section I, can be used to assess at least a partial sequence of the polypeptide or at least a partial identity of the moiety.

In some embodiments, the at least a partial sequence of the polypeptide is assessed using a procedure comprising: a1) providing the polypeptide and the associated polypeptide tag that serves as a recording tag; b1) contacting the polypeptide with a first binding agent capable of binding to the polypeptide, wherein the first binding agent comprises a first coding tag with identifying information regarding the first binding agent; c1) transferring the information of the first coding tag to the recording tag to generate a first order extended recording tag; and d1) analyzing the first order extended recording tag. The step a1) can comprise providing the polypeptide and an associated polypeptide tag joined to a solid support. The method can further comprise contacting the polypeptide with a second (or higher order) binding agent comprising a second (or higher order) binding portion capable of binding to the polypeptide and a coding tag with identifying information regarding the second (or higher order) binding agent, transferring the information of the second (or higher order) coding tag to the first order extended recording tag to generate a second order (or higher order) extended recording tag, and analyzing the second order (or higher order) extended recording tag.

In some embodiments, the at least a partial sequence of the polypeptide is assessed using a procedure comprising: a1) providing the polypeptide and the associated polypeptide tag that serves as a recording tag; b1) contacting the polypeptide with a first binding agent capable of binding to the N-terminal amino acid (NTAA) of the polypeptide, wherein the first binding agent comprises a first coding tag with identifying information regarding the first binding agent; c1) transferring the information of the first coding tag to the recording tag to generate an extended recording tag; and d1) analyzing the extended recording tag. The method can further comprise providing the polypeptide and an associated polypeptide tag joined to a solid support. The method can further comprise contacting the target polypeptide with a second (or higher order) binding agent comprising a second (or higher order) coding tag with identifying information regarding the second (or higher order) binding agent, wherein the second (or higher order) binding agent is capable of binding to a NTAA other than the NTAA of the polypeptide. The contact between the polypeptide with the second (or higher order) binding agent can be conducted in any suitable manner. For example, contacting the polypeptide with the second (or higher order) binding agent can occur in sequential order following the polypeptide being contacted with the first binding agent. In another example, contacting the polypeptide with the second (or higher order) binding agent can occur simultaneously with the poly-peptide being contacted with the first binding agent.

In some embodiments, the at least a partial sequence of the polypeptide is assessed using a procedure comprising: a1) providing the polypeptide and the associated polypeptide tag that serves as a recording tag; b1) contacting the polypeptide with a first binding agent capable of binding to the N-teminal amino acid (NTAA) of the polypeptide, wherein the first binding agent comprises a first coding tag with identifying information regarding the first binding agent; c1) transferring the information of the first coding tag to the recording tag to generate a first order extended recording tag; d1) removing the NTAA to expose a new NTAA of the target polypeptide; e1) contacting the polypeptide with a second (or liluher order) binding agent comprising a second (or higher order) coding tag with identifying information regarding the second (or higher order) bindiiig agent, wherein the second (or higher order) binding agent is capable of binding to the new NTAA, wherein the second (or higher order) binding agent comprises a second coding tag with identifying information regarding the second (or higher order) binding agent; f1) transferring the information of the second (or higher order) coding tag to the first extended recording tag to generate a second order (or higher order) extended recording tag; and g1) analyzing the second order (or higher order) extended recording tag. The steps d1)-g1) can be repeated one or more times. The method can further comprise providing the polypeptide and the associated polypeptide tag joined to a solid support.

In some embodiments, the at least a partial sequence of the polypeptide is assessed using a procedure comprising: a1) providing the polypeptide and the associated polypeptide tag that serves as a recording tag; b1) modifying the N-terminal amino acid (NTAA) of the polypeptide, e.g., with a chemical agent; c1) contacting the polypeptide with a first binding agent capable of binding to the modified NTAA, wherein the first binding agent comprises a first coding tag with identifying information regarding the first binding agent; d1) transferring the information of the first coding tag to the recording tag to generate a first order extended recording tag; and e1) analyzing the first order extended recording tag. The step a1) can comprise providing the polypeptide and the associated polypeptide tag joined to a solid support. The method can further comprise contacting the polypeptide with a second (or higher order) binding agent comprising a second (or higher order) coding tag with identifying information regarding the second (or higher order) binding agent, wherein the second (or higher order) binding agent is capable of binding to a modified NTAA other than the modified NTAA of step b1). The contact between the polypeptide and the second (or higher order) binding agent can be conducted in any suitable manner. For example, contacting the polypeptide with the second (or higher order) binding agent can occur in sequential order following the target polypeptide being contacted with the first binding agent. In another example, contacting the polypeptide with the second (or higher order) binding agent can occur simultaneously with the polypeptide being contacted with the first binding agent.

In some embodiments, analyzing the first order and/or the second (or higher order) extended recording tag also assesses the polypeptide tag.

In some embodiments, the moiety comprises a moiety polypeptide, and at least a partial identity or sequence of the moiety can be assessed using a procedure comprising: a2) providing the moiety polypeptide and the associated moiety tag that serves as a recording tag; b2) contacting the moiety polypeptide with a first binding agent capable of binding to the moiety polypeptide, wherein the first binding agent comprises a first coding tag with identifying information regarding the first binding agent; c2) transferring the information of the first coding tag to the recording tag to generate a first order extended recording tag; and d2) analyzing the first order extended recording tag. The method can further comprise contacting the moiety polypeptide with a second (or higher order) binding agent comprising a second (or higher order) binding portion capable of binding to the moiety polypeptide and a coding tag with identifying information regarding the second (or higher order) binding agent, transferring the information of the second (or higher order) coding tag to the first order extended recording tag to generate a second order (or higher order) extended recording tag, and analyzing the second order (or higher order) extended, recording tag.

In some embodiments, the at least a partial sequence of the moiety polypeptide is assessed using a procedure comprising: a2) providing the moiety polypeptide and the associated moiety tag that serves as a recording tag; b2) contacting the moiety polypeptide with a first binding agent capable of binding to the N-terminal amino acid (NTAA) of the moiety polypeptide, wherein the first binding agent comprises a first coding tag with identifying information regarding the first binding agent; c2) transferring the information of the first coding tag to the recording tag to generate an extended recording tag; and d2) analyzing the extended recording tag. The method can farther comprise providing the moiety polypeptide and an associated moiety tag joined to a solid support. The method can farther comprise contacting the moiety polypeptide with a second (or higher order) binding agent comprising a second (or higher order) coding tag with identifying information regarding the second (or higher order) binding agent, wherein the second (or higher order) binding agent is capable of binding to a NTAA other than the NTAA of the polypeptide. The contact between the moiety polypeptide with the second (or higher order) binding agent can be conducted in any suitable manner. For example, contacting the moiety poiypeptide with the second (or higher order) binding agent can occur in sequential order following the moiety polypeptide being contacted with the first binding agent. In another example, contacting the moiety polypeptide with the second (or higher order) binding agent can occur simultaneously with the moiety polypeptide being contacted with the first binding agent.

In some embodiments, the at least a partial sequence of the moiety polypeptide is assessed using a procedure comprising: a2) providing the moiety polypeptide and the associated moiety tag that serves as a recording tag; b2) contacting the moiety polypeptide with a first binding agent capable of binding to the N-terminal amino acid (NTAA) of the moiety polypeptide, wherein the first binding agent comprises a first coding tag with identifying information regarding the first binding agent; c2) transfeiring the information of the first coding tag to the recording tag to generate a first order extended recording tag; d2) removing the NTAA to expose a new NTAA of the moiety polypeptide; e2) contacting the moiety polypeptide with a second (or higher order) binding agent comprising a second (or higher order) coding tag with identifying information regarding the second (or higher order) binding agent, wherein the second (or higher order) binding agent is capable of binding to the new NTAA, wherein the second (or higher order) binding agent comprises a second coding tag with identifying information regarding the second (or higher order) binding agent; f2) transferring the information of the second (or higher order) coding tag to the first extended recording tag to generate a second order (or higher order) extended recording tag; and g2) analyzing the second order (or higher order) extended recording tag. The steps d2)-g2) can be repeated one or more times. The method can further comprise providing the moiety polypeptide and the associated moiety tag joined to a solid support.

In some embodiments, the at least a partial sequence of the moiety polypeptide is assessed using a procedure comprising: a2) providing the moiety polypeptide and the associated moiety tag that serves as a recording tag; b2) modifying the N-terminal amino acid (NTAA) of the moiety polypeptide, e.g., with a chemical agent; c2) contacting the moiety polypeptide with a first binding agent capable of binding to the modified NTAA, wherein the first binding agent comprises a first coding tag with identifying information. regarding the first binding agent; d2) transferring the information of the first coding tag to the recording tag to generate a first order extended recording tag; and e2) analyzing the first order extended recording tag. The step a2) can comprise providing the moiety polypeptide and the associated moiety tag joined to a solid support. The method can further comprise contacting the moiety polypeptide with a second (or higher order) binding agent comprising a second (or higher order) coding tag with identifying information regarding the second (or higher order) binding agent, wherein the second (or higher order) binding agent is capable of binding to a modified NTAA other than tfte modified NTAA of step b1). The contact between the moiety polypeptide tad the second (or higher order) binding agent can be conducted in any suitable manner. For example, contacting the moiety polypeptide with the second (or higher order) binding agent can occur in sequential order following the moiety polypeptide being contacted with the first binding agent. In another example, contacting the moiety polypeptide with the second (or higher order) binding agent can occur simultaneously with the moiety polypeptide being contacted with the first binding agent.

In some embodiments, analyzing the first order and/or the second (or higher order) extended recording tag also assesses the moiety tag.

In some embodiments, the first order and/or the second (or higher order) extended recording tag comprises a polynucleotide, e.g., DNA or RNA, and at least a partial sequence of the polynucleotide in the first order and/or the second (or higher order) extended recording tag is assessed to assess the at least a partial sequence of polypeptide and/or the moiety, and/or to assess the polypeptide tag and; or the moiety tag. The polynucleotide sequence can be assessed using any suitable techniques or procedures. For example, the polynucleotide sequence can be assessed using Maxam-Gilbert sequencing, a chain-termination method, shotgun sequencing, bridge PCR, single-molecule real-time sequencing, ion semiconductor (ion torrent sequencing), sequencing by synthesis, sequencing by ligation (SOLiD sequencing), chain termination (Sanger sequencing), massively parallel signature sequencing (MPSS), polony sequencing, 454 pyrosequencing, Illumina (Solexa) sequencing, DNA nanobs sequencing, heliscope single molecule sequencing, single molecule real time (SMRT) sequencing, nanopore DNA sequencing, tunnelling currents DNA sequencing, sequencing by hybridization, sequencing with mass spectrometry, microfluidic Sanger sequencing, a microscopy-based technique, RNAP sequencing, or in vitro virus high-throughpat sequencing.

The present methods can use to assess any suitable of s ,vital proximity between polypeptide and a moiety in a sample. In some embodiments, both the polypeptide and the moiety are parts of a larger polypeptide. In some examples, the larger polypeptide has a primary protein structure, and the polypeptide and the moiety are in spatial proximity in the primary protein structure. In some examples, the larger polypeptide has a secondary, tertiary and/or quaternary protein structure(s), and the polypeptide and the moiety are in spatial proximity in the secondary, tertiary and/or quatematy protein structure(s). In other embodiments, the polypeptide and the moiety belong to two different molecules. For example, the polypeptide and the moiety can belong to two different proteins in the same protein complex. In other examples, the moiety can be a part of a polynucleotide molecule, e.g., a DNA or a RNA molecule, that is bound to, complexed with or in close proximity with the polypeptide in the sample. In these embodiments, the present methods can use to assess any suitable type of spatial proximity between or among different molecules, e.g., spatial proximity between or among different subunits in a protein complex, a protein-DNA complex or a protein-RNA complex.

### III. Uses of the Present Methods

The present methods can be used for any suitable purpose. In some embodiments, the present methods can be used to assess spatial relationship between a single polypeptide and a single moiety in a sample. In other embodiments, the present methods can be used to assess spatial relationship between or among a single polypeptide and a plurality of moieties in a sample. In still other embodiments, the present methods can be used to assess spatial relationship between or among a plurality ofpolypeptides and a plurality of moieties in a sample.

In some embodiments, both the polypeptide and the moiety belong to the same molecule, and the present methods are used to identify and/or assess interaction between the polypeptide and the moiety in the same molecule. For example, the moiety can be a moiety amino acid or a moiety polypeptide in the same protein of the polypeptide, and the present methods are used to identity and/or assess interaction between the polypeptide and the moiety amino acid or moiety polypeptide in the protein. In another example, the present methods are used to identify and/or assess interaction regions or domains in the same protein. In still another example, the moiety is a modified moiety amino acid or a modified moiety polypeptide, and the present methods are used to identify and/or assess interaction between the polypeptide and the modified moiety amino acid or the modified moiety polypeptide in the protein. In some embodiments, both the polypeptide and the moiety are parts of a larger polypeptide and the polypeptide and the moiety are in spatial proximity in the secondary, tertiary and/or quaternary protein structure(s).

In some embodiments, the present methods can further comprise preserving the structure of a target molecule, e.g., by cross-linking, before analysis. For example, the target molecule can be a target protein, and the present methods can further comprise preserving the structure of the target protein, eg., by cross-linking, before analysis. In such examples, the present methods can be used to identify arid/or assess disulfide bond(s) in the target protein.

In some embodiments, the moiety belongs to a molecule that is bound, complexed with in close proximity with a target protein that comprises the target potypeptide, and the present methods are used to identify and/or assess interaction between the target protein and the molecule that is bound to, complexed with or in close proximity with the target protein in a sample. For example, the moiety can be a moiety amino acid or a moiety polypeptide in a moiety protein that is bound to, complexed with or in close proximity with a target protein that comprises the target polypeptide, and the present methods are used to identify and/or assess interaction, between the target protein and the moiety protein in a sample. In another example, the present methods are used to identify and/or assess interaction regions or domains in the target protein and the moiety protein that is bound to, complexed with or in close proximity with the target protein, e.g., to identify and/or assess interaction regions or domains involved in protein subunit binding or complexing, or protein-ligand binding or complexing. In still another example, the present methods are used to assess a probability whether two or more polypeptide regions or domains belong to the same protein, the same protein binding pair or the same protein complex.

In some embodiments, the assessing of at least a partial sequence of the polypeptide and at least partial identity of the moiety is performed separately from formilig the linking structure between the polypeptide and moiety. For example, the assessing of at least a partial sequence of the polypeptide and at least partial identity of the moiety is perfamed after forming a linking structure between the polypeptide and the moiety and after the transferring of formation between the polypeptide tag and the moiety tag to form a shared unique molecule identifier and/or barcode. In some examples, the assessing of at least a partial sequence of the polypeptide and at least partial identity of the moiety is performed after the polypeptide is dissociated from the moiety. In some aspects, the assessing of at least a partial sequence of the polypeptide and at least partial identity of the moiety is performed after the polypeptide (with the associated polypeptide tag) is immobilized on a support, and after the moiety (with the associated moiety tag) is immobilized on a solid support. ht some of any such embodiments, the assessing of at least a partial sequence of the polypeptide and at least partial identity of the moiety includes contacting the polypeptide and moiety with one or more binding agents. In some examples, the contacting of the polypeptide and moiety with one or more binding agents is performed: after forming a linking structure between the polypeptide and the moiety and after the transferring of infomiation between the polypeptide tag and the moiety tag to form a shared unique molecule identifier and/or barcode; after the polypeptide is dissociated from the moiety; after the polypeptide (with the associated polypeptide tag) is immobilized on a support and after the moiety (with the associated moiety tag) is immobilized on a solid support.

In some embodiments, the present methods further comprise a physical partitioning step, e.g., partitioning by emulsions or other physical partitioning techniques. In some embodiments, the present methods do not comprise a physical partitioning step.

In some embodiments, the present methods further comprise limiting the number of proteins, e.g., an average number of proteins, in the analysis. The number of proteins in the analysis can be limited by any suitable technique or procedure. For example, the number of proteins can be limited by dilution. In another example, the number of proteins can be limited by binding the proteins to a solid support such as beads. In some embodiments, the immobilization of the pairwise or interacting polypeptide and moiety on a solid support is performed to achieve the desired sampling. In some cases, the immobilization of the polypeptide and the moiety is performed to increase the likelihood that both the polypeptide and moiety are immobilized on the same solid support. In some examples, either the polypeptide or moiety (and its associated tag) is immobilized on a solid support, then the polypeptide is dissociated from the moiety, and the other of the polypeptide or moiety is immobilized on the same solid support (e.g., same bead).

In some embodiments, the present methods can be used to analyze a protein in its native conformation. In some embodiments, the forming of a linking structure between a polypeptide and a moiety are performed on a polypeptide and a moiety in a sample that is interacting or in spatial proximity while each maintains its secondary, tertiary and/or quaternary protein structure(s). In other embodiments, the present methods can be used to analyze a denatured or renatured protein.

In some embodiments, the present methods can be used to analyze a protcome, e.g., an entire proteome. The proteome can be a proteome of a virus, a viral fraction, a cellular fraction, a cellular organelle, a cell, a tissue, an organ, an organism, or a biological sample.

The present methods can be used to assess spatial relationship between a polypeptide and a moiety in any suitable sample. In some embodiments, the present methods can be used to assess spatial relationship between a target polypeptide and a moiety in a biological sample, e.g., a blood, plasma, serum or urine sample.

In some embodiments, the present methods can be conducted homogeneously, e.g., in a solution. In some embodiments, the present methods can be conducted heterogeneously, e.g., in a suspension.

### IV. Kits and Articles of Manufacture for Assessing Spatial Relationship

Provided herein are kits for assessing spatial, relationship between one or more polypeptides and one or more moieties in a sample including using any of the methods provided herein. In one aspect, the kit further comprises instructions describing a method for assessing a sample using the methods provided herein. In some embodiment, provided herein are a kit and components for use in a method for analysing a macromolecule, the method comprising: a) forming a linking structure between, a site of a polypeptide in a sample and a site of a moiety in said sample, said linking structure comprising a polypeptide tag associated with said site of said polypeptide and a moiety tag associated with said site of said moiety, wherein said polypeptide tag and said moiety tag are associated; b) transferring information between said associated polypeptide tag and said moiety tag or ligating said associated polypeptide tag and said moiety tag to form a shared unique molecule identifier (UMI) and/or barcode; c) breaking said linking structure via dissociating said polypeptide from said moiety and dissociating said polypeptide tag from said moiety tag, while maintaining association between said polypeptide and said polypeptide tag, and maintaining association between said moiety and said moiety tag; and d) assessing said polypeptide tag and at least a partial sequence of said polypeptide, and assessing said moiety tag and at least a partial identity of said moiety, wherein said assessed portions of said polypeptide tag and said moiety tag comprise: aid shared unique molecule identifier (UMI) and/or barcode indicates that said site of said polypeptide and said site of said moiety in said sample are in spatial proximity.

In some embodiment, provided herein are a kit and components for use in a method for assessing identity and spatial relationship between a polypeptide and a moiety, the method comprising: a) forming a linking structure between a site of a polypeptide in a sample and a site of a moiety in said sample, said linking structure comprising a polypeptide tag associated with said site of said polypeptide and a moiety tag associated with said site of said moiety, wherein said polypeptide tag arid said moiety tag are associated; b) transferring information between said associated polypeptide tag and said moiety tag to form a shared unique molecule identifier (UMI) and/or barcode, wherein the shared UMI and/or barcode is formed as a separate record polynucleotide; c) breaking said linking structure via dissociating said polypeptide from said moiety and dissociating said polypeptide tag from said moiety tag, while maintaining association between said polypeptide and said polypeptide tag, and maintaining association between said moiety and said moiety tag; d) assessing said polypeptide tag and at least a partial sequence of said polypeptide, and assessing said moiety tag and at least a partial identity of said moiety; and e) assessing said separate record polynucleotide to establish the spatial relationship between the site of the polypeptide and the site of the moiety.

In some embodiments, provided herein are a kit and components for use in a method for providing a pre-assembled structure comprising a shared unique molecule identifier (UMI) and/or bareode iti the middle portion flanked by a polypeptide tag on one side and a moiety tag on the other side; b) forming a linking structure between a site of a polypeptide in a sample and a site of a moiety in said sample by associating said polypeptide tag of said pre-assembled structure to said site of said polypeptide and associating said moiety tag of said pre-assembled structure to said site of said moiety; c) breaking said linking structure via dissociating said polypeptide from said moiety and dissociating said polypeptide tag from said moiety tag, while maintaining association between said polypeptide and said polypeptide tag, and maintaining association between said moiety and said moiety tag; and d) assessing said polypeptide tag and at least a partial sequence of said polypeptide, and assessing said moiety tag and at least a partial identity of said moiety, wherein said assessed portions of said polypeptide tag and said moiety tag comprise said shared unique molecule identifier (UMI) and/or barcode indicates that said site of said polypeptide and said site of said moiety in said sample are in spatial proximity.

In some embodiments, the kits provided herein include components for performing the methods for assessing spatial interaction and/or relationship, reaction mixture compositions that comprise the components as well as to kits for constructing such reaction mixtures.

In some embodiments, the kit comprises one or more polypeptide taes and one or more moiety tags; reaaents for forming a linking structure between a polypeptide and a moiety in a sample; and reagents for assessing the identity of the moiety and at least a partial sequence of the polypeptide. In some embodiments, the kit further comprises instructions for assessing identity and spatial relationship between a polypeptide. In some embodiments, the kit comprises instructions for preparing the sample. In some embodiments, the kit comprises components, such as polypeptides and polynucleotides as described in section I and II.

In some embodiments, the kit comprises one or more polypeptide tags and one or more moiety tags; reagents for forming a linking structure between a polypeptide and a moiety in a sample, wherein the linking structure is formed as a separate record polynucleotide; and reagents for assessing the identity of the moiety and at least a partial sequence of the polypeptide. In some of any of the provided embodiments, the kit further comprises reagents for analyzing the separate record polynucleotide.

In some of any of the provided embodiments, the kit further comprises one or more reagents for ligation (e.g., an enzymatic or chemical ligation, a splint ligation, a sticky end ligation, a single-strand (ss) ligation such as a ssDNA ligation, or any combination thereof), or a polymerase-mediated reaction (e.g., primer extension of single-stranded nucleic acid or double-stranded nucleic acid), or any combination thereof. In some embodiments, the ligation reagent is a chemical ligation reagent or a biological ligation reagent, for example, a ligase, such as a DNA ligase or RNA ligase for ligating single-stranded nucleic acid or double-stranded nucleic acid, or (ii) a reagent for primer extension of single-stranded nucleic acid or double-stranded nucleic acid, optionally wherein the kit further comprises a ligation reagent comprising at least two ligases or variants thereof (e.g., at least two DNA ligases, or at least two RNA ligases, or at least one DNA ligase and at least one RNA ligase), wherein the at least two ligases or variants thereof comprises an adenylated ligase and a constitutively non-adenylated ligase, or optionally wherein the kit further comprises a ligation reagent comprising a DNA or RNA ligase and a DNA/RNA deadenylase.

In some embodiments, the kit comprises reagents for assessing the identity of the moiety and at least a partial sequence of the polypeptide. In some cases, the kit comprises a library of binding agents, wherein each binding agent composes a binding moiety and a coding polymer comprising identifying information regarding the binding moiety. In some embodiments, the binding moiety is capable of binding to one or more N-terminal, internal, or C-terminal amino acids of the fragment, or capable of binding to the one or more N-terminal, internal, or C-terminal amino acids modified by a functionalizing reagent.

In some embodiments, the kit comprises reagents for providing a polypeptide associated directly or indirectly with a polypeptide tag and for providing a moiety associated directly or indirectly with a moiety tag; a reagent for functionalizing the N-terminal amino acid (NTAA) of the polypeptide; a first binding agent comprising a first binding portion capable of binding to the functionalized NTAA and a first coding tag with identifying information regarding the first binding agent, or a first detectable label; and a reagent for transferring the information of the first coding tag to the recording tag to generate an extended recording tag. In some embodiments, the kit further comprises a reagent for analyzing the extended recording tag or a reagent for detecting the first detectable label.

In some embodiments, the kit additionally comprises a reagent for eliminating the frinctionalized NTAA to expose a new NTAA. Any suitable removing reagent can be used. In some embodiments, the removed amino acid is an amino acid modified using any of the methods or reagents provided herein. For example, the reagent may comprise an enzymatic or chemical reagent to remove one or more terminal amino acid. For example, in some cases, the reagent for eliminating the functionalized NTAA is a carboxypeptidase, aminopeptidase, dipeptidyl peptidase, dipeptidyl aminopeptidase, or variant, mutant, or modified protein thereof; hydrolase or variant, mutant, or modified protein thereof; mild Edman degradation; Edmanase enzyme; TFA, a base; or any combination thereof. In some cases, the removing reagent comprises trifittoroacetic acid or hydrochloric acid. In some examples, the removing reagent comprises acylpeptide hydrolase (APH). In some embodiments, the removing reagent includes a carboxypeptidase or an aminopeptidase or a variant, mutant, or modified protein thereof; a hydrolase or a variant, mutant, or modified protein thereof; a mild Edman degradation reagent: an Edmanase enzyme; anhydrous TFA, a base; or any combination thereof. In some embodiments, the mild Edman degradation uses a dichloro or monochloro acid; the mild Edman degradation uses TFA, TCA, or DCA; or the mild Edman degradation uses triethylamine, triethanolamine, or triethylammonium acetate (Et₃NHOAc).

In some cases, the reagent for removing the amino acid comprises a base. In some embodiments, the base is a hydroxide, an alkylated amine, a cyclic amine, a carbonate buffer, trisodium phosphate buffer, or a metal salt. In some examples, the hydroxide is sodium hydroxide; the alkylated amine is selected from methylarnine, ethylamine, propylamine, dimethylarnine, diethylamine, dipropylamine, trimethylamine, triethylamine, tripropylamine, cyclohexylarnine, beuzylamine, aniline, diphenylamine, N,N-Diisopropylethylamine (DIPEA), and lithium diisopropylamide (LDA), the cyclic amine is selected from pyridine, pyrimidine, imidazole, pyrrole, indole, piperidine, orolidine, 1,8-diazabicyclo[5.4.0]undec-7-ene (DBU), and 1,5-diazabicyclo[4.3.0]non-5-ene (DBN); the carbonate buffer comprises sodium carbonate, potassium carbonate, calcium carbonate, sodium bicarbonate, potassium bicarbonate, or calcium bicarbonate; the metal salt comprises silver; or the metal salt is AgClO₄.

In some embodiments, the method further includes contacting the polypeptide with a peptide coupling reagent. In some embodiments, the peptide coupling reagent is a carbodiimide compound. In some examples, the carbodiimide compound is diisopropyicarbodiimide (DIC) or 1-ethyl-3-(3-dimethylaminopropyl)carbodiimide (EDC).

In one aspect, the kit farther comprises buffers for use with the provided methods. In some examples, the kit farther comprises a detergent or a surfactant. In some embodiments, the provided kits include buffers used for information transfer between the polypeptide tag and the moiety tag, for extension of polynucleotides, for a primer extension reaction, and/or for ligation reactions. In one aspect the kit ftrther comprises one or more solutions or buffers (e.g., Tris, MOPS, etc.) for performing a method according to any of the methods of the invention.

In any of the preceding embodiments, the kit can comprise a support or a substrate, such as a rigid solid support, a flexible solid support, or a soil solid support, and including a porous support or a non-porous support.

In any of the preceding embodiments, the kit can comprise a support which comprises a bead, a porous bead, a porous matrix, an array, a surface, a glass surface, a silicon surface, a plastic surface, a slide, a filter, nylon, a chip, a silicon wafer chip, a flow through chip, a biochip including signal transducing electronics, a well, a microtitre well, a plate, an ELISA plate, a disc, a spinning interferometry disc, a membrane, a nitrocellulose membrane, a nitrocellulose-based polymer surface, a nanoparticle (e.g., comprising a metal such as magnetic nanoparticles (Fe₃O₄), gold nanoparticles, and/or silver nanoparticles), quantum dots, a nanoshell, a nanocage, a microsphere, or any combination thereof. In one embodiment, the support comprises a polystyrene bead, a polymer bead, an agarose bead, an acrylamide bead, a solid core bead, a porous bead, a paramagnetic bead, glass bead, or a controlled pore bead, or any combination thereof. In some embodiments, the support or substrate comprises a plurality of spatially resolved attachment points.

In any of the provided embodiments, the kit can comprise a support and/or can be for analyzing a plurality of the analytes (such as polypeptides), in sequential reactions, in parallel reactions, or in a combination of sequential and parallel reactions. In one embodiment, the analytes are spaced apart on the support at an average distance equal to or greater than about 10 nm, equal to or greater than about 15 nm, equal to or greater than about 20 nm, equal to or greater than about 50 nm, equal to or greater than about 100 nm, equal to or greater than about 150 nm, equal to or greater than about 200 nm, equal to or greater than about 250 nm, equal to or greater than about 300 nm, equal to or greater than about 350 nm, equal to or greater than about 400 nm, equal to or greater than about 450 nm, or equal to or greater than about 500 nm.

In some embodiments, the kit further comprises one or more vessels or containers, e.g., tube vessels (e.g., test tube, capillary, Eppendorf tube) useful for performing the method of use. In some examples, the components are each provided in separate containers.

In one aspect the kit further comprises one or more oligonucleotides, and in one aspect (optionally) free nucleotides, and in one aspect (optionally) sufficient free nucleotides to carry out a PCR reaction, a rolling circle replication, a ligase-chain reaction, a reverse transcription, a nucleic acid labeling or tagging reaction, or derivative methods thereof

In one aspect the kit further comprises at least one enzyme, wherein in one aspect (optionally) the enzyme is a polytnerase. In one aspect the kit further comprises one or more oligonucleotides, free nucleotides and at least one polymerase or enzyme capable of amplifying a nucleic acid in a PCR reaction, a rolling circle replication, a ligase-chain reaction, a reverse transcription or derivative methods thereof. The one or more oligonucleotides can specifically hybridize to a nucleic acid from a sample from a subject, (e.g. from an animal, a plant, an insect, a yeast, a virus, a phage, a nematode, a bacteria or a fungi).

In some embodiments, the kit further comprises reagents and components for purifying, isolating, and/or collecting the polypeptides, moieties, tags, and/or polynucleotides (e.g. separate record polynucleotides). In some embodiments, the kit further comprises reagents for concatenating and collecting the polypeptides, moieties, tags, and/or polynucleotides (e.g. separate record polynucleotides). In some embodiments, the kit further includes instructions for preparing the sample. In some cases, the kit comprises reagents and components for nucleic acid (e.g. DNA or RNA) isolation, precipitation, and/or collection.

### Exemplary Embodiments

Among the provided embodiments are:
1. A method for assessing identity and spatial relationship between a polypeptide and a moiety in a sample, which method comprises:
a) forming a linking structure between a site of a polypeptide in a sample and a site of a moiety in said sample, said linking structure comprising a polypeptide tag associated with said site of said polypeptide and a moiety tag associated with said site of said moiety, wherein said polypeptide tag and said moiety tag are associated;
b) transferring information between said associated polypeptide tag and said moiety tag or ligating said associated polypeptide tag and said moiety tag to forma shared unique molecule identifier (UMI) and/or barcode;
c) breaking said linking structure via dissociating said polypeptide ftorn said moiety and dissociating said polypeptide tag from said moiety tag, while maintaining association between said polypeptide and said polypeptide tag, and maintaining association between said moiety and said moiety tag; and
d) assessing said polypeptide tag and at least a partial sequence of said polypeptide, and assessing said moiety tag and at least a partial identity of said moiety,
wherein said assessed portions of said polypeptide tag and said moiety tag comprise said shared unique molecule identifier (UMI) and/or barcode indicates that said site of said polypeptide and said site of said moiety in said sample are in spatial proximity.
2. The method of embodiment 1, wherein the moiety comprises a polypeptide.
3. The method of embodiment 1, wherein the moiety comprises a polynucleotide.
4. The method of any one of embodiments 1-3, wherein the polypeptide tag comprises a polynucleotide.
5. The method of any one of embodiments 1-4, wherein the moiety tag comprises a polynucleotide.
6. The method of embodiment 5, wherein the polypeptide tag comprises a first polynucleotide and the moiety tag comprise a second polynucleotide, the first and second polynucleotides comprise a complementary sequence, and the polypeptide tag and the moiety tag are associated via the complementary sequence.
7. The method of embodiment 6, wherein transferring infortntition between the associated polypeptide tag and moiety tag comprises extending both the first polymacIeotide of the polypeptide fag and the second polynucleotide of the moiety tag to form the shared UMI and/or barcode.
8. The method of embodiment 6, wherein transferring information between the associated polypeptide tag and moiety tag comprises extending one of the first polynucleotide of the polypeptide tag and the second polynucleotide of the moiety tag to form the shared UMI and/or barcode.
9. The method of embodiment 5, wherein the polypeptide tag comprises a double-stranded polynucleotide and the moiety tag comprise a doubic-strandcd polynucleotide, and transferring information between the associated polypeptide tag and moiety tag comprises ligating the double-stranded polynucleotides to form the shared UMI and/or barcode.
10. The method of embodiment 9, wherein the shared UMI and/or barcode comprises sequences of both, the double-stranded polynucleotides.
11. The method of embodiment 9, wherein the shared UMI and/or barcode comprises sequence of one of the double-stranded polynucleotides.
12. The method of any one of embodiments 1-11, wherein, in the linking structure, the polypeptide tag and the moiety tag are associated stably.
13. The method of any one of embodiments 1-11, wherein, in the linking structure, the polypeptide tag and the moiety tag are associated transiently.
14. The method of any one of embodiments 1-13, wherein, in the linking structure, the polypeptide tag and the moiety tag are associated directly.
15. The method of any one of embodiments 1-13, wherein, in the linking structure, the polypeptide tag and the moiety tag are associated indirectly, e.g., via a linker or UMI between the polypeptide tag and the moiety tag.
16. A method for assessing identity and spatial relationship between a polypeptide and a moiety in a sample, which method comprises:
a) forming a linking structure between a site of a polypeptide in a sample and a site of a moiety in said sample, said linking structure comprising a polypeptide tag associated with said site of said polypeptide and a moiety tag associated with said site of said moiety, wherein said polypeptide tag and said moiety tag are associated;
b) transferring information between said associated polypeptide tag and said moiety tag to form a shared unique molecule identifier (UMI) and/or barcode, wherein the shared UMI and/or barcode is formed as a separate record polynucleotide;
c) breaking said linking structure via dissociating said polypeptide from said tnoiety and dissociating said polypeptide tag from said moiety tag, while maintaining association between said polypeptide and said polypeptide lag. and maintaining association between said moiety and said moiety tag;
d) assessing said polypeptide tag and at least a partial sequence of said polypeptide, and assessing said moiety tag and at least a partial identity of said moiety; and
e) assessing said separate record polynucleotide to establish the spatial relationship between the site of the polypeptide and the site of the moiety.
17. The method of embodiment 16, wherein the polypeptide tag and the moiety tag comprise polynucleotides.
18. The method of embodiment 16 or embodiment 17, wherein the linking structure is formed between the polypeptide tag and the moiety tag via the separate record polynucleotide.
19. The method of any one of embodiments 16-18, wherein the method forms multiple separate record polypeptides between the polypeptide tag and more than one site of said moiety or more titan one moiety.
20. The method of any one of embodiments 16-19, wherein step e) establishes the spatial relationship between the site of the polypeptide and two or more sites of said moiety or two or more moieties.
21. The method of any one of embodiments 16-20, wherein, in the linking structure, the polypeptide tag and the separate record polynucleotide are associated transiently.
22. The method of any one of embodiments 16-21, wherein, in the linking structure, the polypeptide tag and the separate record polynucleotide are associated directly.
23. The method of any one of embodiments 16-22, wherein, in the Unking structure, the moiety tag and the separate record polynucleotide are associated transiently.
24. The method of any one of embodiments 16-23, wherein, in the linking structure, the moiety tag and the separate record polynucleotide are associated directly.
25. The method of any one of embodiments 16-24, wherein the separate record polynucleotide is formed by extension, e.g., primer extension.
26. The method of any one of embodiments 16-24, wherein the separate record polynucleotide is formed by ligation.
27. The method of any one of embodiments 16-26, wherein the separate record polynucleotide is released from said polypeptide tag and said moiety tag.
28. The method of any one of embodiments 16-27, further comprising collecting said separate record polynucleotide prior to assessing said separate record polynucleotide.
29. The method of embodiment 28, wherein assessing said separate record polynucleotide comprises sequencing said collected shared unique molecule identifier (UMI) and/or barcode, thereby producing sequencing data.
30. The method of any one of embodiments 16-29, further comprising concatenating said collected separate record polynucleotides prior to assessing said separate record polynucleotide.
31. The method of embodiment 30, wherein assessing said separate record polynucleotide comprises sequencing said concatenated separate record polynucleotides.
32. The method of any one of embodiments 1-31, wherein in forming the linking structure, a single polypeptide tag is associated with a single site of the polypeptide, a single polypeptide tag is associated with a plurality of sites of the poiypeptide, or a plurality of the polypeptide tags are associated with a plurality of sites of the polypeptide.
33. The method of any one of embodiments 1-32, wherein in forming the linking structure, a single moiety tag Is associated with a single site of the moiety, a single moiety tag is associated with a plurality of sites of the moiety, or a plurality of the moiety tags are associated with a phiraiity of sites of the moiety.
34. The method of any one of embodiments 1-33, wherein transferring information between the associated polypeptide tag and tlve moiety tag or iigating the associated polypeptide rag and the moiety tag lorms a single shared unique molecule identifier (UMI) and/or barcode.
35. The method of embodiment 34, wherein the single shared unique molecule identifier (UMI) and/or barcode is formed by combining multiple sequences, e.g., multiple UMIs and/or barcodes from the polypeptide tag and/or the moiety tag.
36. The method of any one of embodiments 1-33, wherein transferring information between the associated polypeptide tag and the moiety tag or ligating the associated polypeptide tag and the moiety tag forms a plurality of shared unique molecule identifiers (UMI) and/or barcodes.
37. The method of any one of embodiments 1-36, wherein, in the linking structure, the shared UMI and/or barcode comprises a complementary polynucleotide hybrid, and dissociating the polypeptide tag from the moiety tag comprises denaturing the complementary polynucleotide hybrid.
38. The method of any one of embodiments 1-37, wherein both the polypeptide and the moiety are parts of a larger polypeptide, and dissociating the polypeptide from the moiety comprises fragmenting tlte larger polypeptide into peptide fragments.
39. The method of embodiment 38, wherein the larger polypeptide is fragmented into peptide fragments by a protease digestion.
40. The method of any one of embodiments 1-39, wherein the moiety is a part of a molecule that is bound to, complex ed with or in close proximity with t:lte polypeptide in the sample.
41. The method of embodiment 40, wherein the polypeptide and the moiety belong to two different proteins in the same protein complex.
42. The method of embodiment 40, wherein the moiety is a pan of a polynucleotide molecule that is bound to, complcxed with or in close proximity with the polypeptide in the sample.
43. The method of any one of embodiments 1-42, wherein the at least a partial sequence of the polypeptide is assessed using a procedure comprising:

a1) providing the polypeptide and the associated polypeptide tag that serves as a recording tag;

b1) contacting the polypeptide with a first binding agent capable of binding to the polypeptide, wherein the first binding agent comprises a first coding tag with identifying information regarding the first binding agent;

c1) transferring the information of the first coding tag to the recording tag to generate a first order extended recording tag; and

d1) analyzing the first order extended recording tag.
44. The method of embodiment 43, wherein analyzing the first order extended recording tag also assesses the polypeptide lag.
45. The method of any one of embodiments 1-44, wherein the moiety comprises a moiety polypeptide, and at least a partial identity of the moiety is assessed using a procedure comprising:

a2) providing the moiety polypeptide and the associated moiety tag that serves as a recording tag;

b2) contacting the moiety polypeptide with a first binding agent capable of binding lo the moiety polypeptide, wherein the first binding agent comprises a first coding tag with identifying information regarding the first binding agent;

c2) transferring the information of the first coding tag to the recording tag to generate a first order extended recording tag; and

d2) analyzing the first order extended recording tag.
46. The method of embodiment 45, wherein analyzing the first order extended recording tag also assesses the moiety tag.
47. A method for assessing identity and spatial relationship between a polypeptide and a moiety in a sample, which method comprises:
a) providing a pre-assembled structure comprising a shared unique molecule identifier (UMI) and/or barcode in the middle portion flanked by a polypeptide tag on one side and a moiety tag on the other side;
b) forming a linking structure between a site of a polypeptide in a sample and a site of a moiety in said sample by associating said polypeptide tag of said pre-assemblcd structure to said site of suid polypeptide and associating said moiety tag of said pre-assemblcd structure to said site of said moiety;
c) breaking said linking structure via dissociating said polypeptide from said moiety and dissociating said polypeptide mg from said moiety tag, while maintaining association between said polypeptide and said polypeptide tag, and maintaining association between said moiety and said moiety tag; and
d) assessing said polypeptide tag and at least a partial sequence of said polypeptide, and assessing said moiety tag and at least a partial identity of said moiety,
wherein said assessed portions of said polypeptide tag and said moiety tag comprise said shared unique molecule identifier (UMI) and/or barcode indicates that said site of said polypeptide and said site of said moiety in said sample are in spatial proximity.
48. The method of embodiment 47, wherein the moiety comprises a polypeptide.
49. The method of embodiment 47, wherein the moiety comprises a polynucleotide.
50. The method of any one of embodiments 47-49, wherein the polypeptide tag comprises a polynucleotide.
51. The method of any one of embodiments 47-50, wherein the moiety tag comprises a polynucleotide.
52. The method of any one of embodiments 47-51, wherein, in the linking structure, the polypeptide tag and the moiety tag are associated stably.
53. The method of any one of embodiments 47-51, wherein, in the linking structure, the polypeptide tag and the moiety tag are associated transiently.
54. The method of any one of embodiments 47-53, wherein, in the linking structure, the polypeptide tag and the moiety tag are associated directly.
55. The method of any one of embodiments 47-53, wherein, in the linking structure, the polypeptide tag and the moiety tag are associated indirectly, e.g., via a linker or UMI between the polypeptide tag and the moiety tag.
56. The method of any one of embodiments 47-55, wherein in forming the linking structure, a single polypeptide tag is associated with a single site of the polypeptide, a single polypeptide tag is associated with a plurality of sites of the polypeptide, or a plurality of the polypeptide tags are associated with a plurality of sites of the polypeptide.
57. The method of any one of embodiments 47-56, wherein in forming the linking structure, a single moiety tag is associated with a single site of the moiety, a single moiety tag is associated with a plurality of sites of the moiety, or a plurality of the moiety tags are associated with a plurality of sites of the moiety.
58. The method of any one of embodiments 47-57, wherein the formed linking structure comprises a single shared unique molecule identifier (UMI) and/or barcode.
59. The method of any one of embodiments 47-57, wherein the formed linking structure comprises a plurality of shared unique molecule identifiers (UMI) and/or barcodes.
60. The method of any one of embodiments 47-57, wherein the polypeptide tag comprises a first polynucleotide and the moiety tag comprise a second polynucleotide.
61. The method of any one of embodiments 47-60, wherein, in the linking structure, the shared UMI and/or barcode comprises a complementary polynucleotide hybrid, and dissociating the polypeptide tag from the moiety tag comprises denaturing the complementary polynucleotide hybrid.
62. The method of any one of embodiments 47-61, wherein both the polypeptide and the moiety are parts of a larger polypeptide, and dissociating the polypeptide from die moiety comprises fragmenting the larger polypeptide into peptide fragments.
63. The method of cmhodiment 62, wherein the larger polypeptide is fragmented into peptide fragments by a protease digestion.
64. The method of any one of embodiments 47-63, wherein the moiety is a part of a molecule that is bound to, complexed with or in close proximity with the polypeptide in the sample.
65. The method of embodiment 64, wherein the polypeptide and the moiety belong to two different proteins in the same protein complex.
66. The method of embodiment 64, wherein the moiety is a part of a polynucleotide molecule that is bound to, complexed with or in close proximity with the polypeptide in the sample.
67. The method of any one of embodiments 47-66, wherein the at least a partial sequence of the polypeptide is assessed using a procedure comprising:

a3) providing the polypeptide and the associated polypeptide tag that serves as a recording tag;

b3) contacting the polypeptide with a first binding agent capable of binding to the polypeptide, wherein the first binding agent comprises a first coding tag with identifying information regarding the firel binding agent;

c3) transferring the information of the first coding tag to the recording tag to gcncraic a first order extended recording tag; and

d3) analyzing the first order extended recording tag.
68. The method of embodiment 67, wherein analyzing the first order extended recording tag also assesses the polypeptide tag.
69. The method of any one of embodiments 47-68, wherein the moiety comprises a moiety polypeptide, and at least a partial identity of the moiety is assessed using a procedure comprising:

a4) providing the moiety polypeptide and the associated moiety tag that serves as a recording tag;

b4) contacting the moiety polypeptide with a first binding agent capable of binding to the moiety polypeptide, wherein the first binding agent comprises a first coding tag with identifying information regarding the first binding agent;

c4) transferring the information of the first coding tag to the recording tag to generate a first order extended recording tag; and

d4) analyzing the first order extended recording tag.
70. The method of embodiment 69, wherein analyzing the first order extended recording tag also assesses the moiety tag.
71. The method of any one of embodiments 1-70, wherein the assessing of at least a partial sequence of the polypeptide and at least partial identity of the moiety Is performed after forming the linking structure between the site of the polypeptide and the site of the moiety.
72. The method of any one of embodiments 1-71, wherein the assessing of at least a partial sequence of the polypeptide and at least partial identity of the moiety is performed after the polypeptide is dissociated from the moiety.
73. The method of any one of embodiments 43-46 and 67-70, wherein the contacting of the polypeptide anti the moiety with one or more binding agents is performed after forming a linking structure between the polypeptide and the moiety.
74. The method of any one of embodiments 43-46, 67-70, and 73, wherein the contacting of the polypeptide and the moiety with one or more binding agents is performed after the polypeptide is dissociated from the moiety.
75. A kit for assessing identity and spatial relationship between a polypeptide and a moiety in a sample, comprising:
(a) one or more polypeptide tags and one or more moiety tags;
(b) reagents for forming a linking structure between a polypeptide and a moiety in a sample; and
(c) reagents for assessing the identity of the moiety and at least a partial sequence of the polypeptide.
76. A kit for assessing identity and spatial relationship between a polypept ide and a moiety in a sample, comprising:
(a) one or more polypeptide tags and one or more moiety tags;
(b) reagents for forming a linking structure between a polypeptide and a moiety in a sample, wherein the linking structure is formed as a separate record polynucleotide; and
(c) reagents for assessing the identity of the moiety and at least a partial sequence of the polypeptide.
77. The kit of embodiment 76, further comprising one or more reagents for analyzing the separate record poly-nucleotide.
78. The kit of any one of embodiments 75-77, wherein the reagents for assessing the identity of the moiety and at least a partial sequence of the poiypeptide comprises a library of binding agents, wherein each binding agent comprises a binding moiety and a coding polymer comprising identifying information regarding the binding moiety, wherein the binding moiety is capable of binding to one or more N-terminal, internal, or C-terminal amino acids of the fragment, or capable of binding to the one or more N-terminal internal, or C-terminal amino acids modified by a functionalizing reagent.
79. A kit for assessing spatial relationship, comprising:
(a) a reagent for providing a poiypeptide associated directly or indirectly with a polypeptide tag and for providing a moiety associated directly or indirectly with a moiety tag;
(b) a reagent for functionalizing the N-terminal amino acid (NTAA) of the polypeptide;
(c) a first binding agent comprising a first binding portion capable of binding to the functionalized NTAA and (c1) a first coding tag with identifying information regarding the first binding agent, or (c2) a first detectable label; and
(d) a reagent for transferring the information of the first coding tag to the recording tag to generate an extended recording tag; and optionally
(e) a reagent for analyzing the extended recording tag or a reagent for detecting the first detectable label.
80. The kit of embodiment 79, wherein the kit additionally comprises a reagent for eliminating the functionalized NTAA to expose a new NTAA.
81. The kit of embodiment 80, wherein the reagent for eliminating the tiincrionalized NTAA is a carboxypeptidase or aminopeptidasc or variant, mutant, or modified protein thereof; a hydrolase or variant, mutant, or modified protein thereof; mild Edman degradation; Edmanasc enzyme; TFA, a base: or any combination thereof.
82. The kit of any of embodiments 75-79, further comprising a support or substrate.
83. The kit of embodiment 82, wherein the support or substrate is a bead, a porous bead, a porous matrix, an array, a glass surface, a silicon surface, a plastic surface, a filter, a membrane, nylon, a silicon wafer chip, a flow through chip, a biochip including signal transducing electronics, a microtitre well, an ELISA plate, a spinning interferometry disc, a nitrocellulose membrane, a nitrocellulose-based polymer surface, a nanoparticle, or a microsphere.
84. The kit of embodiment 82 or embodiment 83, wherein the support or substrate comprises a plurality of spatially resolved attachment points.

### EXAMPLES

The following examples are offered to illustrate but not to limit the methods, compositions, and uses provided herein.

### Example 1: Pairwise Association

In this example, peptide 1 (Pep 1) and peptide 2 (Pep 2) are subsequences of Protein 1. DNA tags containing is Is are covalently attached to sites in a protein sample. The sites should be appropriately spaced on average so as to optimize yield of useful information per the assay design.

DNA tag with UMI 1 is linked to Pep 1 and DNA tag with UMI 2 is linked to Pep 2 in the protein sample. The DNA tags are designed so that UMI sequences can be copied from one tag to another, via universal complementary 3′ ends utilized as primers by DNA polymerase. A reaction that copies tag information is carried out, e.g., one cycle of annealing+extension with DNA polymerase. (See e.g., Assarsson, Lundberg et al. 2014.) By virtue of proximity, UMI 1 and UMI 2 write to each other. In some examples, only a single cycle of extension is carried out, so as to form unique tag pairs. Other variations are possible, in which a sequence is propagated across multiple tags. Such a system should be designed so that undesired tag multimers are not generated or at least minimized.

Next, Protein 1 is cleaved and peptide-UMI-tag-pairs are processed to generate NGPS data. The DNA tags incorporating UMIs are used as recording tags (or written to recording tags) in the NGPS assay. Following NGS sequencing and sequence analysis, the following sequence constructs are extracted:
{Pep1, UMI1-UMI2}
{Pep2, UMI2-UMI1}
   Provided that UMI 1 I and UMI 2 are to a first approximation “unique” (i.e., having a suitably low probability of occurring multiple times in the sample by chance), we can use this information to deduce with high confidence that Pep 1 and Pep 2 are in close proximity in the protein sample. Particularly if we empirically tune and calibrate the system so that there is a high likelihood that peptides linked using Partitioning By Association (PBA) are part of the same protein, we can infer that Pep 1 and Pep 2 are likely subsequences of a single protein. This additional, information is not obtained from NGPS alone. When, combined with the peptide sequence data, it allows us to identify protein sequences with higher confidence because we can search for coincident pairs (or more) of peptide sequence matches.

### Example 2: Network Reconstruction

There is no requirement that peptide pairs be from the same protein. In some examples, the PBA process is applied to a complex protein sample. The sample is labeled with DNA tags and UMI pairs are formed as described in Exainple 1. In some cases, UMI pairs will associate subsequences of a protein (cis-protein associations or CPAs). In other cases, UMI pairs will form between proteins (trans-protein associations or TPAs). In a complex protein sample there can be a mix of CPAs and TPAs.

Even with just a single CPA per protein, PBA significantly increases the ability to uniquely identify a protein. However, additional power is gained by reconstructing networks of pairs. For example, if Pep 3 and Pep 4 are subsequences of Protein 2. Let us assume that PBA associates:
Pep 1 from Protein 1 with Pep 3 from Protein .2
Pep 2 from Protein 1 with Pep 4 from Protein 2.
   Let us assume that we can map Pep 1 and Pep 2 to Protein 1, but we can't map Pep 3 and Pep 4 to Protein1. However, we can infer that Pep 3 and Pep 4 have a reasonable likelihood of belonging to the same protein (or a small subset of proteins that were in proximity to Protein 1). Therefore, we can use this “partitioning” information to identify high-likelihood matches, and bootstrap together a network of pairwise relationships that allows us identify proteins using PBA using shorter and less accurate sequences than would be required without PBA.

PBA can be used together with physical partitioning. However, because of this ‘network’ effect, often no physical partitioning is required, PBA can be carried out in bulk without the need for emulsions, or other complex partitioning techniques. Instead, “virtual” proximity-based partitions are established at the molecular level and reconstructed informatically.

In some examples, it is preferable to limit the number of proteins that are in sufficiently close proximity to generate pairwise codes, preferably, PBA would generate many relatively discrete “networks” rather than one large, diffuse network that in principle could comprise the entire protein sample. Simple methods of limiting the average number of proteins assmiated together include dilution and physical separation, e.g., by adsorption or other attachment to a solid support such as beads.

### Example 3: Labeling of Proteins and Protein Complexes with DNA Tags

A DNA tag comprised of common primer sequences flanking a UMI/barcode and 5′ conjugation moiety (for coupling directly or indirectly to polypeptide) enables coupling to native proteins or protein complexes. A number of standard bioconjugation methods (e.g., Hermanson 2013) can be employed to couple the DNA tag directly to reactive amino acid residues (e.g., Lys, Cys, Tyrosine, etc., see Ref), or indirectly via a heterobiftinctional linker. For instance heterobifunctional linkers, such as NHS-PEG11-mTet can be used to chemically label lysine residues in a buffet such as 50 mM sodnam borate or HEPES (pH 8.5), and gelierate an orthogonal chemical “click” group for subsequent coupling to a DNA tag, with a 5′ tran-cyclo octane (TCO) group. After lysine labeling with NHS-PEG11-mTet, excess NHS-PEG11-mTet linker is removed using a 10k MWCO filter or reverse phase purification resin (RP-S).

A 5′ TCO labeled DNA tag is coupled to the mTet-labeled proteins in 1× PBS buffer (pH 7.5). Excess DNA tag can be removed by scavenging on an mTet scavenger resin. After removal of excess DNA tag, a proximity-based primer extension step is used to transfer information between proximal DNA tags. Specifically, proximal DNA tags are allowed to anneal in Extension buffer (50 mM Tris-Cl (pH 7.5), 2 mM MgSO4, 125 μM dNTPs, 50 mM NaCl, 1 mM ditinothreitod, 0.1% Tween-20, and 0.1 mg/mL, BSA) for 5 minutes at room temp after a brief 2 min. heating step to 45° C. After annealing, Klenow exo-DNA polymerase (NEB, 5 U/μL), is added to the beads for a final concentration of 0.125 U/μl, and incubated at 23° C. for 5 min. After primer extension, the reaction is quenched by adding urea to 8 M to denature protein and protein. complexes.

### Example 4: Processing of Proximity DNA Tagged Polyeptide

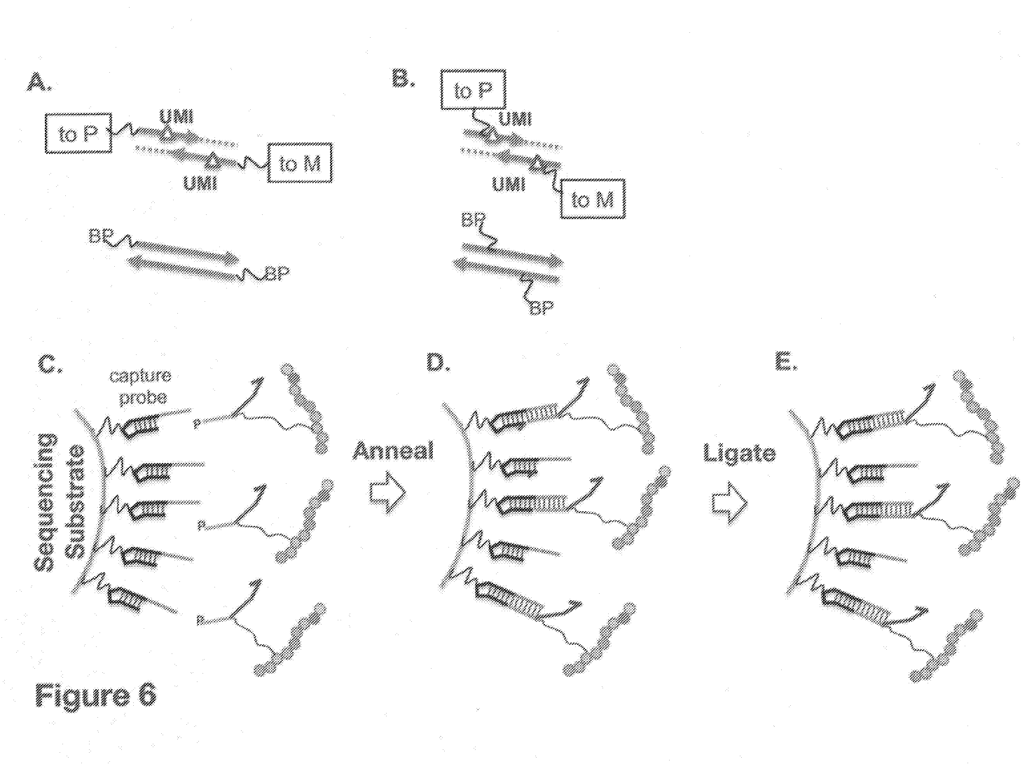

After primer extension and protein denaturation, the denatured polypeptides are acylated at remaining unreacted cysteine or lysine residues, and then subject to protease digestion with an endopeptidase like trypsin, LysC, ArgC, etc. The proximity-extended DNA tags on the labeled peptides act as a recording tags in our NGPS ProteoCode assay as described in PCT/US2017/030702. The DNA tagged peptides are immobilized onto a sequencing subsuute (e.g., beads) by direct chemical conjugation or by hybridization capture and ligation to DNA capture probes directly attached to sequencing substrate (See e.g., FIG. 6).

After attachment of fhe DNA-peptide constructs to the sequencing substrate, at least two species of DNA tags are present (see e.g., FIG. 5C), one DNA tag type is comprised of a 3′ Sp1′ sequence, and the other DNA tag type is comprised of a 3′ Sp2′ sequence. These two sequence types are converted into a universal Sp spacer sequence by annealing conversion primers (Sp2-Sp′ and Sp1-Sp1). Extension upon these primers sequence generates the final recording tag for ProteoCode sequencing.

### Example 5: Ligation Based Proximity Cycling

This Example describes a method for assessing proximity interaction of a polypeptide and one or more moieties using ligation based proximity cycling. The poiypeptide and moieties are each labeled with a DNA tag. The DNA tags are designed to interact by cycling extension, ligation, and denaturation.

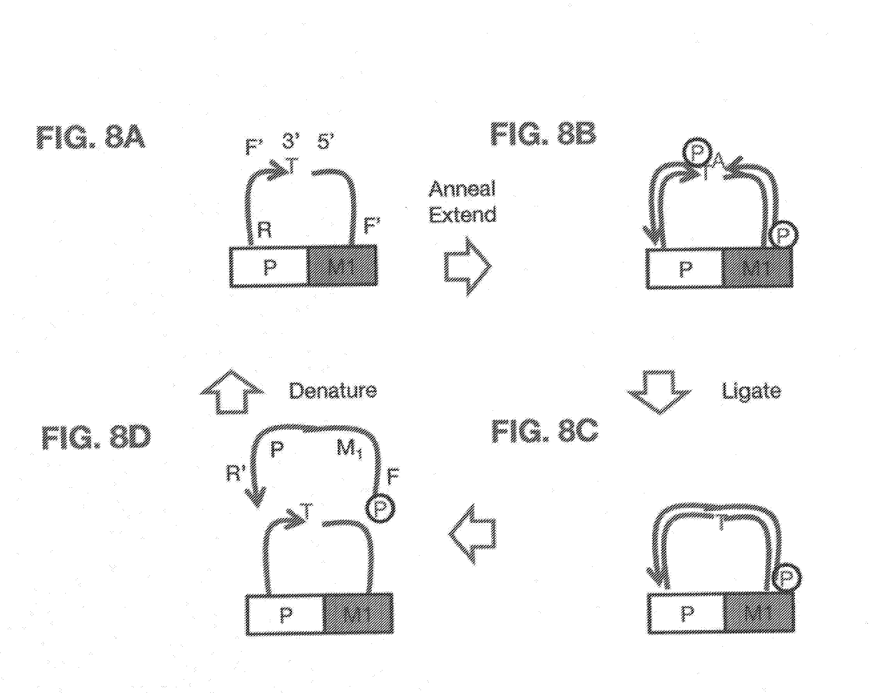

In the first step of a given cycle, a common primer anneals to the F′ site on the 3′ end of the DNA tags. The DNA tag on the polypeptide is oriented with its 3′ end away from the polypeptide and an extra T base, and the DNA tags on the moieties is oriented such that it 3′end is attached to the moiety and the 5′ end is free (FIG. 8A). In some embodiments, the design can be reversed. After annealing of F primers to the DNA tags (polypeptide tag and moiety tag), primer extension generates double stranded DNA tag products, and A extendase activity of the polymerase generates an A overhang on the double stranded DNA tag product annealed to the moiety's DNA tag (FIG. 8B). This A overhang on the moiety tag and the T overhang on the, polypeptide tag enables ligation (FIG. 8C). The 5′ end of the moiety DNA tag is non-phosphorylated and non-ligatable, whereas the 5′ end of the F primer is phosphorylated and ligatable. As shown in FIG. 8D, ligation produces a separate record polynucleotide of P-M₁. In some cases, the polypeptide is in spatial proximity of more than one moiety (e.g., M1, M2, etc.). Cyclic annealing, extension, and ligation generates multiple linear records of P-M₁, P-M₂, etc. (e.g. separate record polynucleotides) (FIG. 9A-9B). Indirect or overlapping information from multiple separate record polynucleotides further indicates spatial proximity information for the polypeptide with two or more moieties (FIG. 9C).

Cyclic annealing, extension, and ligation are performed a follows: A 50 μl reaction comprised of 100 ng of DNA tagged protein complexes in 1× Ext-Lig buffer (20 mM Tris-HCl pH 8.0, 25 mM potassium acetate, 2 mM magnesium acetate, 1 mM NAD, 200 μM dNTPs except for dATP at 500 μM, 10 mM DTT, 0.1% Triton X-100), 200 nM F primer, 0.5 U Tag polymerase (NEB), and 2 U Pfu DNA ligase (D540K mutant) (U.S. Pat. No. 5,427,930; Tanabe et al., Archaea (2015) 2015:267570). The reaction is cycled for 30 cycles under the following conditions: 94° C. for 2 min, then 60° C. 1 min, 40° C. 5 min, 94° C. 30 s for 30 cycles. After extension ligation thermocycling in the presence of F primer, the resultant records are PCR amplified using F and R primer using standard PCR conditions.

The proximity of P to neighboring M₁, M₂, etc. can be determined using the provided method. The sequences or identities of and M₁, M₂ moieties are further determined using ProteoCode sequencing (e.g., International Patent Application Publication No. WO 2017/192633),

### Example 6. Concatenation of DNA Libraries for Nanopore Sequencing

DNA libraries were PCR amplified (20 cycles) with 5′ phosphorylated primers using VeraSeq 2.0 Ultra DNA polymerase to generate library amplicons suitable for blunt end ligation (˜20 ng/μL PCR yield). To concatenate PCR products, 20 μL of PCR reaction was mixed with 20 μL 2× Quick Ligase buffer and 1 μL, Quick Ligase (NEB) and incubated at room temperature for ˜16 hrs. The resultant ligated product, ˜0.5-2 kb in length (probably a mix of some circular products as well), was purified using a Zymo purification column and eluted into 20 μL water. The resultant concatenated product was prepared for nanopore sequencing using a Rapid Sequencing Prep kit (SQK-RAD002) which uses transposase-based adapter addition and analyzed on a MinION Mk 1B (R9.4) device. Other methods of concatenation DNA libraries include the method described by Schlecht et al. using Gibson assembly and can also be employed for concatenating DNA libraries as described above and used in nanopore sequencing (Schlecht et al., (2017) Sci Rep 7(1): 5252).

### Example 7. Labeling of Peptides and Information Transfer Between Proximal Molecules

This example describes information transfer in a proximity model system between two portions of a polypeptide: a biotin containing portion of the peptide (moiety) and a phenylalanine (F) containing portion of the peptide (peptide).

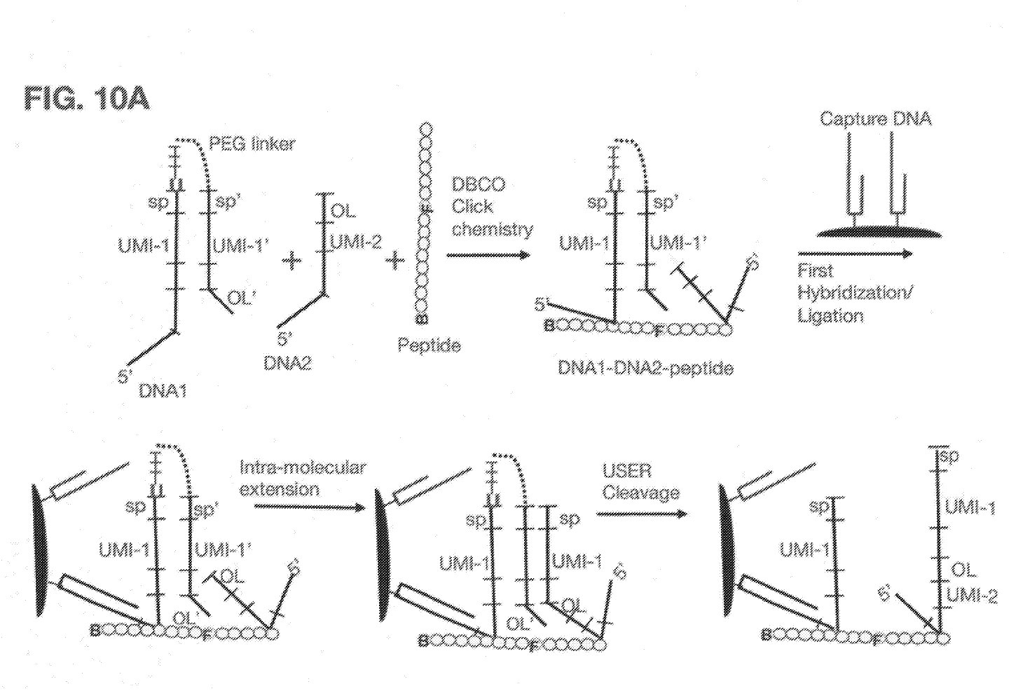

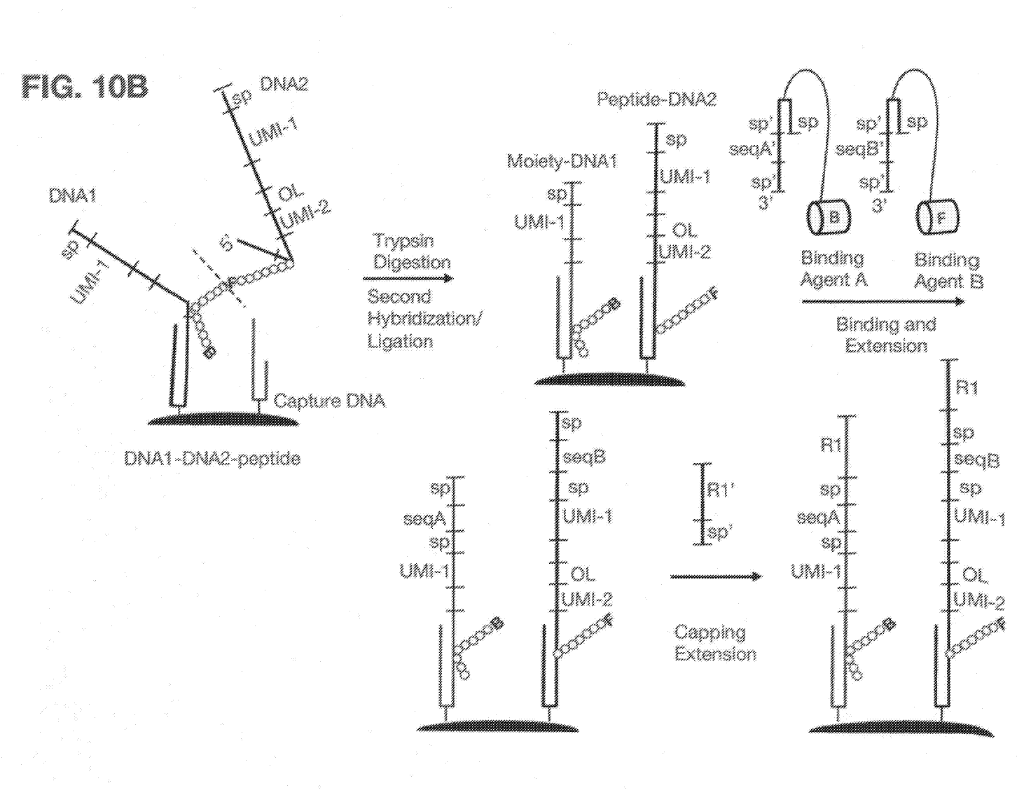

A polypeptide tag (DNA1) comprising complementary spacer regions (sp′ and sp), a PEG linker, and complementary UMI sequences (UMI1 and UMI1′) as shown in FIG. 10A were prepared by extension and ligation of synthetic oligonucleotides. The 3′ end of DNA1 comprised an overlay region (OL′) that is complementary to an OL region on DNA2 (peptide tag).

The moiety tag (DNA1) and peptide tag (DNA2) were linked to the model polypeptide (K(Biotin)GSGSK(N3)GSGSRFAGVAMPGAEDDVVGSGS-K(N3)-NH2 as set forth in SEQ ID NO: 1) which contained a biotin at the N-terminus and an internal phenylalanine. The DNA1 and DNA2 tags were linked with the peptide using a DBCO click reaction, in which DNA1 (5 uM), DNA2 (5 uM) and the peptide (1 μM) were mixed in 100 mM HEPES (pH 7.5) and 150 mM NaCI buffer and heated at 60° C. overnight. Because each peptide has two sites for DNA attachment, three different products were generated: a peptide with two DNA1 attached, a peptide with two DNA2 attached, or a peptide with DNA1 and DNA2 attached. Only peptide attached to both DNA1 and DNA2 contained the necessary hybridization region for information transfer. To remove free excess DNA, streptavidin beads (MyOne Streptavidin T1, Thermo Fisher, USA) were used to isolate polypeptide complexes with DNA via binding with the biotin. Twenty (20) μL of the reaction mixture were incubated with streptavidin beads (10 μL) at 25° C. for 40 min. After removal of the supernatant and washing twice with PBS+0.1% tween 20, the samples were eluted in 20 μL of 95% formamide at 60° C. for 5 min. As a control, a DNA3 oligo was incubated with a peptide that was the same as SEQ ID NO:1 except it contained only 1 azide group). The DNA3-peptide complex was made by incubation at 60° C. for overnight to generate a control complex and was purified as previously described. Attachment of the DNA to the polypeptides before and after purification was confimied by mobility shift on a 15% denaturing polyacrylamide (TBU) gel.

The purified DNA1-DNA2-peptide complexes were captured on magnetic sepharose beads via DNA1 by hybridization and ligation of DNA1 to the bead-attached DNA1 capture DNA (FIG. 10A). By design, the beads comprised two types of capture DNAs, one with a region complementary to DNA1 and the other with a region complementary to DNA2. However, hybridization sites for DNA2 were pre-blocked with complementary single stranded DNA, to enable capture via DNA1. Equal concentration of purified DBCO click reaction mixture, containing DNA1-DNA2-peptide and DNA3-peptide (total concentration: 0.1 nM) were mixed and hybridized with the magnetic sepharose beads in a buffer with 5× SSC, 0.02% SDS and 15% formamide, followed by washing with PBS+0.1% tween 20 and ligation. After the ligation, un-ligated substrate and the capture DNA blocker for DNA2 were washed away by 0.1 M NaOH+0.1% tween 20.

For information transfer between DNA1 and DNA2, 0.125 U/μL Klenow fragment (3′→5′ exo-) (KF⁻) was used in presence of dNTP mixture (125 μM for each), 50 mM Tris-HCl (pH, 7.5), 2 mM MgSO₄, 50 mM NaCl, 1 mM DTT, 0.1% Tween 20, and 0.1 mg/mL BSA. The reaction was incubated at 37° C. for 5 min to perform infra-molecular extension of DNA2 using DNA1 as a template.

After information transfer, the linking structure between DNA1 and DNA2 (the polypeptide and moiety tags) was broken by cleaving at the single uracil (U) present (FIG. 10A). The cleavage reaction comprised 0.05 U/μL USER Enzyme, 0.2 U/μL T4 PNK. 1 mM ATP, 5 mM DTT in presence of 1× CutSmart buffer from NEB, incubated at 37° C. for 60 min. Next, trypsin digestion was conducted to separate the peptide from the moiety (in this example, the F containing portion of the model polypeptide and biotin containing portion of the model polypeptide, respectively) as shown in FIG. 10B. Digestion was performed at 37° C. for 2 h with 0.02 mg/mL Trypsin, 0.1% tween 20, 500 mM NaCI, and 50 mM HEPEs (pH, 8.0). During the trypsin cleavage reaction, separated moiety-DNA2, was re-captured by hybridization to bead-attached DNA2 capture DNA. After washing with PBS+0.1% Tween, the samples were incubated in the quick ligase mixture as earlier described for the first ligation at 25° C. for 30 min to covalently link the moiety-DNA2 with the bead-attached DNA2 capture DNA.

A final capping step was performed by adding an oligo (R1′-sp′) to a KF⁻ reaction mixture as described earlier with the beads in the presence of dNTPs (125 μM each) to generate the final products with the cap sequence (R1) at the 3′ end for both DNA1 and DNA2 as shown in FIG. 10B. R1 and another DNA region (at the 5′ of DNA1 and DNA2) were used as the annealing sites for adapter PCR for NGS. After amplification and introduction of binding sites and index sequences by adapter and index PCR, the samples were sequenced by MiSeq Reagent Kit v3 (Illumina, USA). Amplicons were sequenced using a MiSeq and counted.

Results demonstrating information transfer are shown in Table 1. An average of 491 information transfer events were detected in replicate experiments (Replicate 1=617, Replicate 2=365). Events were detected by identifying unique UMI-1 matches between DNA1 and DNA2, corresponding to unique pairings between individual peptide-DNA1 and moiety-DNA2 constructs.

TABLE 1
Information transfer results

| | Molecular | Replicate | Replicate |
|---|---|---|---|
| Metric | Markers Analyzed | 1 (counts) | 2 (counts) |
| Number of DNA1- | unique UMI-1 found | 36513 | 23984 |
| containing | DNA1 | | |
| complexes analyzed | | | |
| Number of DNA2- | unique UMI-2 found | 2078 | 1213 |
| containing | DNA2 | | |
| complexes analyzed | | | |
| Number of | unique UMI-1 found | 617 | 365 |
| information | both in DNA1 and | | |
| transfer events | DNA2 | | |

To detect the background for this experiment, the control sample DNA3-peptide was mixed with DNA1-DNA2-peptide in equal ratio during the first hybridization/ligation step. The NGS output ratio of DNA3 and DNA2 was equal to or less than 0.0066, indicating that almost all the information transfer events happened within the same molecule in FIG. 10B.

In summary, this example demonstrates that the information transfer between the peptide and the moiety (Biotin and F-containing portions of the peptide) in the model polypeptide was effective with low background.

In some cases, the polypeptide and moiety are assessed for at least a partial sequence of the polypeptide and at least a partial identity of the moiety (FIG. 10B) prior to the final capping step described above. An encoding step is performed to assess at least a portion of the sequence of the peptide. Binding agents with a coding tag align containing information regarding the binding agent can recognize the N-terminal amino acids or recognize a portion of the polypeptide or moiety. After the binding agent binds to their corresponding target, the 3′-spacer′ region of the coding tag hybridizes to the 3′-spacer of the DNA align linked with the same peptide. The peptide-linked DNA can be elongated by copying the coding tag by extension using KF⁻, as a result, transferring the information from the coding tag to the DNA sequence linked to the peptides DNA1 and DNA2) for analysis.

The encoding step is then followed by the final step of capping as described above wherein an oligo containing a universal priming sequence (R1′-sp′) is added into a KF⁻ reaction mixture with the peptides (associated with DNA1 and DNA2) in presence of dNTPs (e.g., 125 μM each) to generate a final product for NGS readout.

### Example 8. Assessment of Encoding Function Using a Mixture of Binding Agents

This example describes an exeinplary encoding assay performed using binding agents that recognize a portion of the peptide (e.g., an N-terminal amino acid).

In an exemplary model system for assessing at least a portion of a polypeptide and moiety a peptide comprising a phenylalanine (F-peptide) anached to DNA recording tag and a biotin attached to DNA recording tag were assessed in an encoding assay. A binder that does not bind biotin or N-terminal phenylalanine (F) on a peptide was also included as a negative control. Two hundred (200) of an exemplary binding agent that binds phenylalanine when it is the N-terminal amino acid residue (F-binder), 44 nM of a mono-streptavidin binder that recognizes biotin (mSA-binder), and 200 nM the negative control binder were incubated with biotin linked to a recording tag and F-peptide (F at the N-terminal) linked to a recording tag. The binding agents, each linked with corresponding coding lags identifying the binding agent, were ineubated with beads conjugated with biotin-recording tag conjugates and F-peptide-recording tag conjugates. Following binding and washing, the transfer of coding tag information to recording tags by extension was effected by incubating the beads in a solution Containing 0.125 units/μL Klenow fragment (3′→5′ exo-) (MCLAB, USA), dNTP mixture (125 μM for each), 50 mM Tris-HCl (pH, 7.5), 2 mM MgSO₄, 50 mM NaCI, 1 mM DTT, 0.1% Tween 20, and 0.1 mg/mL BSA. The reaction was incubated at 37° C. for 5 min. The beads were washed after encoding. The extended recording tags of the assay were subjected to PCR amplification and analyzed by next-generation sequencing (NGS).

As shown by the NGS results in Table 2, the mSA and F-binders were able to bind and encode their corresponding targets and the tested hinders exhibited low encoding signal for the peptide that is not the target of the binding agent.

TABLE 2
Encoding yield for mSA binder and F binder

| | Biotin on DNA | F-Peptide |
|---|---|---|
| Binding Agent | (Encoding Fraction) | (Encoding Fraction) |
| mSA Binder | 0.354 | 0.017 |
| F Binder | 0.004 | 0.103 |
| Negative Control Binder | 0.004 | 0.003 |

### Exemplary Advantages

There is no requirement for each peptide derived from a single protein (or physical partition) to have the same barcode as other peptides from that protein (or physical partition). Every site (even within the same protein) can have a different sequence identifier e.g., a UMI. Proteins can be handled in bulk, with no beads etc, required. A solid support can be used for convenience &/or to help facilitate, but in principle the process can be done in solution on arbitrarily complex samples. For example, an entire proteome sample can be partitioned in bulk. The heavy lifting is done computationally instead.

When conducted on nave proteins in complexes, PBA can be used for reconstruction of protein complexes. When conducted on renatumi proteins, PBA can be used to identify proteins that have a propensity to associate.

PBA can be used to associate other types of molecule, e.g., DNA-protein complexes. PBA can be used with sample barcodes so that multiple samples can be pooled and analyzed together.

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the show-detailed description, in general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES CITED

US 2015/0224466 A1;
US 2010/0136544 A1;
U.S. Pat. No. 9,029,085 B2;
U.S. Pat. No. 9,085,798 B2;
U.S. Pat. No. 6,511,809 B2;
WO 2017/192633 A1;
WO 2016/123419A1;
WO 2015/070037 A2;
WO 2016/130704 A2;
WO 2017/075265 A1;
WO 2016/061517 A2;
WO 2015/042506 A1;
WO 2016/0138086 A1;
Abe, H., Y. Kondo, H. Jinn ei, N. Abe, K. Furukawa, A. Uchiyarna, S. Tsimeda, K. Aikawa, I. Matsumoto and Y. Ito (2008). “Rapid DNA chemical ligation for amplification of RNA and DNA signal.” Bioconjug Chem 19(1): 327-333;
Assarsson, E., M. Lundberg, G. Holmquist, J. Ejorkesten, S. B. Thorsen, D. Ekraan, A. Eriksson, E. Rennel Dickens, S. Ohlssoa, G. Edfeldt, A. C. Andersson, P. Lindstedt, J. Stenvang, M. Gullberg and S. Fredrikssen (2014). “Homogenous 96-plex PEA immunoassay exhibiting high sensitivity, specificity, and excellent scalability.” PLoS One 9(4): e95192;
El-Sagheer, A, H., V. V. Cheong and T. Brown (2011). “Rapid chemical ligation of oligonucleotides by the Diels-Alder reaction.” Org Biomol Chem 9(1); 232-235;
El-Sagheer, A. H, A. P. Sanzone, R. Gao, A. Tavassoli and T. Brown (2011). “Biocompatible artificial DNA linker that is read through by DNA polymerases and is functional in Escherichia coli,” Proc. Nati Acad Sci USA 108(28); 11338-11343;
Hermanson, G. (2013). Bioconjugation Techniques, Academic Press;
Holding, A. N. (2015). “XL-MS: Protein cross-linking coupled with mass spectrometry” Methods 89: 54-63;
Kilpatrick, L. E. and E. L. Kilpatrick (2017). “Optimizing High-Resolution Mass :Spectrometry for the Identification of Low-Abundance Post-Translational Modifications of Intact Proteins.” J. Proteome Res 16(9): 3255-3265;
Park, J., M. Koh, J. Y. Koo, S. Lee and S. B. Park (2016). “Investigation of Specific Binding Proteins to Photoaffinity Linkers for Efficient Deconvolution of Target Protein.” ACS Chem Biol 11(1): 44-52;
Schaus, T. E., et al. (2017). “A DNA nanoscope via auto-cycling proximity recording.” Nat Commun 8(1): 696.
Schneider, M., A. BeIsom and J. Rappsilber (2018). “Protein Tertiary Structure by Crosslinking/Mass Spectrometry.” Trends Biochern Sci 43(3): 157-169; and
Switzar, L., M. Giera and W. M. Niessen (2013). “Protein digestion: an overview of the available techniques and recent developments.” J Proteome Res 12(3): 1067-1077.

1. A method for assessing identity and spatial relationship between a polypeptide and a moiety in a sample, which method comprises:
a) forming a linking structure between a site of a polypeptide in a sample and a site of a moiety in said sample, said linking structure comprising a polypeptide tag associated with said site of said polypeptide and a moiety tag associated with said site of said moiety, wherein said polypeptide tag and said moiety tag are associated;
b) transferring information between said associated polypeptide tag and said moiety tag or ligating said associated polypeptide tag and said moiety tag to form a shared unique molecule identifier (UMI) and/or barcode;
c) breaking said linking structure via dissociating said polypeptide from said moiety and dissociating said polypeptide tag from said moiety tag, while maintaining association between said polypeptide and said polypeptide tag, and maintaining association between said moiety and said moiety tag; and
d) assessing said polypeptide tag and at least a partial sequence of said polypeptide, and assessing said moiety tag and at least a partial identity of said moiety,
wherein said assessed portions of said polypeptide tag and said moiety tag comprise said shared unique molecule identifier (UMI) and/or barcode indicates that said site of said polypeptide and said site of said moiety in said sample are in spatial proximity.

2. The method of claim 1, wherein the moiety comprises a polypeptide.

3. The method of claim 1, wherein the moiety comprises a polynucleotide.

4. The method of claim 1, wherein the polypeptide and moiety are dissociated from each other and immobilized on a support prior to assessing at least a partial sequence of the polypeptide and at least partial identity of the moiety.

5. The method of claim 1, which further comprises fragmenting said polypeptide or said moiety by a protease digestion during or after step c) and before step d).

6. The method of claim 1, wherein the polypeptide tag comprises a first polynucleotide and the moiety tag comprise a second polynucleotide, the first and second polynucleotides comprise a complementary sequence, and the polypeptide tag and the moiety tag are associated via the complementary sequence.

7. The method of claim 6, wherein transferring information between the associated polypeptide tag and moiety tag comprises extending both the first polynucleotide of the polypeptide tag and the second polynucleotide of the moiety tag to form the shared UMI and/or barcode .

8. The method of claim 1, wherein assessing said polypeptide tag and said moiety tag comprises sequencing said shared unique molecule identifier (UMI) and/or barcode, and assessing at least a partial identity of said moiety comprises obtaining at least a partial sequence of the moiety.

9. The method of claim 1, wherein the polypeptide tag comprises a double-stranded polynucleotide and the moiety tag comprise a double-stranded polynucleotide, and transferring information between the associated polypeptide tag and moiety tag comprises ligating the double-stranded polynucleotides to form the shared UMI and/or barcode.

10-15. (canceled)

16. The method of claim 1 and the method further comprising assessing said separate record polynucleotide to establish the spatial relationship between the site of the polypeptide and the site of the moiety.

17-39. (canceled)

40. The method of claim 1, wherein the moiety is a part of a molecule that is bound to, complexed with or in close proximity with the polypeptide in the sample.

41-42. (canceled)

43. The method of claim 1, wherein the polypeptide tag and the at least a partial sequence of the polypeptide is assessed using a procedure comprising:
a1) providing the polypeptide and the associated polypeptide tag that serves as a recording tag;
b1) contacting the polypeptide with a first binding agent capable of binding to the polypeptide, wherein the first binding agent comprises a first coding tag with identifying information regarding the first binding agent;
c1) transferring the information of the first coding tag to the recording tag to generate a first order extended recording tag; and
d1) analyzing the first order extended recording tag.

44-46. (canceled)

47. A method for assessing identity and spatial relationship between a polypeptide and a moiety in a sample, which method comprises:
a) providing a pre-assembled structure comprising a shared unique molecule identifier (UMI) and/or barcode in the middle portion flanked by a polypeptide tag on one side and a moiety tag on the other side;
b) forming a linking structure between a site of a polypeptide in a sample and a site of a moiety in said sample by associating said polypeptide tag of said pre-assembled structure to said site of said polypeptide and associating said moiety tag of said pre-assembled structure to said site of said moiety;
c) breaking said linking structure via dissociating said polypeptide from said moiety and dissociating said polypeptide tag from said moiety tag, while maintaining association between said polypeptide and said polypeptide tag, and maintaining association between said moiety and said moiety tag; and
d) assessing said polypeptide tag and at least a partial sequence of said polypeptide, and assessing said moiety tag and at least a partial identity of said moiety,
wherein said assessed portions of said polypeptide tag and said moiety tag comprise said shared unique molecule identifier (UMI) and/or barcode indicates that said site of said polypeptide and said site of said moiety in said sample are in spatial proximity.

48. The method of claim 47, wherein the moiety comprises a polypeptide or a polynucleotide.

49. (canceled)

50. The method of claim 48, which further comprises fragmenting said polypeptide or said moiety by a protease digestion during or after step c) and before step d).

51. The method of claim 47, wherein assessing said polypeptide tag and said moiety tag comprises sequencing said shared unique molecule identifier (UMI) and/or barcode; and assessing at least a partial identity of said moiety comprises obtaining at least a partial sequence of the moiety.

52-63. (canceled)

64. The method of claim 47, wherein the moiety is a part of a molecule that is bound to, complexed with or in close proximity with the polypeptide in the sample.

65-66. (canceled)

67. The method of claim 47, wherein the polypeptide tag and the at least a partial sequence of the polypeptide is assessed using a procedure comprising:
a3) providing the polypeptide and the associated polypeptide tag that serves as a recording tag;
b3) contacting the polypeptide with a first binding agent capable of binding to the polypeptide, wherein the first binding agent comprises a first coding tag with identifying information regarding the first binding agent;
c3) transferring the information of the first coding tag to the recording tag to generate a first order extended recording tag; and
d3) analyzing the first order extended recording tag.

68-74. (canceled)

75. A kit for assessing identity and spatial relationship between a polypeptide and a moiety in a sample, comprising:
(a) one or more polypeptide tags and one or more moiety tags;
(b) reagents for forming a linking structure between a polypeptide and a moiety in a sample; and
(c) reagents for assessing the identity of the moiety and at least a partial sequence of the polypeptide.

76-77. (canceled)

78. The kit of claim 75, wherein the reagents for forming a linking structure comprises ligation reagent or a reagent for primer extension; and the reagents for assessing the identity of the moiety and at least a partial sequence of the polypeptide comprises a library of binding agents, wherein each binding agent comprises a binding moiety and a coding polymer comprising identifying information regarding the binding moiety, wherein the binding moiety is capable of binding to one or more N-terminal, internal, or C-terminal amino acids of a polypeptide, or capable of binding to the one or more N-terminal, internal, or C-terminal amino acids modified by a functionalizing reagent.

79-84. (canceled)
